SEQUENCE LISTING (1 sequence)

SEQ ID NO: 1 - protein, 5 aa, synthetic construct
GGGGS

(19) United States
(12) Patent Application Publication
(10) Pub. No.: US 2026/0265345 A1
(43) Pub. Date: Sep. 10, 2026

(54) RECOMBINANT POLYCLONAL PROTEINS TARGETING HEPATITIS B VIRUS (HBV) AND METHODS OF USE THEREOF

(71) Applicant: GigaGen, Inc., San Carlos, CA (US)

(72) Inventors: Adam Shultz ADLER, Belmont, CA (US); Sheila KEATING, San Francisco, CA (US); Rena Aviva MIZRAHI, Pacifica, CA (US); Ashley Kristine GRAS, San Francisco, CA (US); Michael ASENSIO, Mountain View, CA (US)

(21) Appl. No.: 19/537,350

(22) Filed: Feb. 11, 2026

Related U.S. Application Data
(63) Continuation of application No. PCT/US2024/042286, filed on Aug. 14, 2024.
(60) Provisional application No. 63/519,827, filed on Aug. 15, 2023.

(51) Int. Cl. C07K 16/082; A61K 9/00; A61K 39/00; A61P 31/20

(52) U.S. Cl. CPC C07K 16/082; A61K 9/0019; A61P 31/20; A61K 2039/505

(57) ABSTRACT

Provided herein are pharmaceutical compositions comprising recombinant polyclonal antibodies for treatment of hepatitis B. Specifically, recombinant polyclonal antibodies specific to HBV antigens are provided. Also provided are the methods of making a pharmaceutical composition comprising the recombinant polyclonal antibodies.

### 1. CROSS-REFERENCE FOR RELATED APPLICATIONS

This application is a continuation of International Application No. PCT/US2024/042286, filed Aug. 14, 2024, which claims the benefit of U.S. Provisional Application No. 63/519,827, filed Aug. 15, 2023, each of which is incorporated by reference in its entirety.

### 2. SEQUENCE LISTING

The instant application contains a Sequence Listing which has been submitted via Patent Center and is hereby incorporated by reference in its entirety. Said XML copy, created May 15, 2026, is named 28152-65510US_SEQLISTING.xml, and is 2,045 bytes in size.

### 3. FIELD

Provided herein are recombinant polyclonal antibody composition, also called Recombinant HBV Immune Globulin (rHBIG), recombinant polyclonal antibody proteins, recombinant hyperimmune globulins, or simply recombinant hyperimmunes, with binding specificity for Hepatitis B virus (HBV). Included are therapeutics, vaccines, and libraries, and compositions comprising such antibodies, including pharmaceutical compositions. Also provided are methods of making the antibodies, and methods of using the antibodies, for example, for therapeutic purposes.

### 4. BACKGROUND

Hepatitis B is a serious liver infection caused by the hepatitis B virus (HBV). Although there is a vaccine to prevent HBV, HBV remains a global health problem. HBV can be acute and later become chronic, leading to other diseases and health conditions, such as cirrhosis, hepatocellular carcinoma, and pancreatic cancer.

The World Health Organization (WHO) announced in 2017 that nearly 300 million people were living with HBV infection, with more than 800,000 deaths, and 1.5 million additional infections occurring every year despite the presence of a highly efficacious vaccine (WHO Report 2017). An effective cure for HBV infection remains elusive due to the persistent, self-replenishing viral genome, defective innate and adaptive immune responses, and integration of HBV-DNA into the host genome (Choi 2022). Pegylated interferon-alpha (PEG-IFNa) and NAs are the main first-line treatments for HBV infection. They are highly effective (especially NAs) in suppressing HBV-DNA and normalizing alanine aminotransferase (ALT) (Lampertico 2017; Terrault 2018). In addition, currently approved drugs have led to a reduced risk of developing HCC and associated mortality (Lin 1999; Seto 2017; Thiele 2013). However, due to high relapse rates after finite treatment with NAs, most patients receiving NAs require prolonged, sometimes lifelong therapy (Seto 2015; Lai 2020). While the existing therapies have been developed to block viral replication, immune modulators and antibody drugs or small molecule drugs will be required to eliminate antigen to achieve functional cure.

Accordingly, there is a need in the art for more effect therapeutics for HBV.

### 5. SUMMARY

Many diseases caused by infectious viruses with many variants or serotypes, such as HBV, are best treated by drugs that target multiple epitopes. An established therapeutic modality is multispecific (multivalent) antibodies derived from human or animal plasma, such as intravenous immunoglobulin (IVIG). Polyclonal antibody drugs with higher potency, known as hyperimmune globulins, are often derived from the plasma of recently vaccinated human donors, for example, HepaGam B against HBV.

Plasma-derived antibody therapeutics have substantial drawbacks. First, demand for normal and convalescent donor plasma often outstrips supply. Plasma-derived drugs have suffered from impurities, including infectious viruses and clotting factors, that have resulted in serious adverse events. Antibody drugs derived from animal plasma occasionally cause allergic reactions, lead to antidrug antibodies and have suboptimal effector properties. Because they are derived from naturally occurring proteins, plasma-derived drugs are not easily engineered; for example, it is not possible to modify Fc sequences to improve mechanism of action or drug half-life. Finally, each batch of plasma-derived drug is usually derived from a different cohort of human donors or animals, resulting in batch-to-batch variation.

Many of these problems can be solved by generating multivalent hyperimmune globulins using recombinant DNA technology. However, this strategy presents substantial technical hurdles. Most importantly, a recombinant hyperimmune globulin technology would have to isolate significant numbers of B cells from donors or animals, natively paired heavy and light chain immunoglobulin at a single-cell level, and then clone the sequences into recombinant expression libraries for manufacturing. Conventionally, most production cell lines for recombinant antibody drugs were generated by random integration of expression constructs into mammalian cell genomes.

The present disclosure provides a Recombinant HBV Immune Globulin (rHBIG) generated by a microfluidics and molecular genomics strategy. The general strategy was previously used to create recombinant multivalent hyperimmune globulins against severe acute respiratory syndrome coronavirus-2 (SARS-CoV-2) or Zika virus. The method generated diverse mixtures of thousands of recombinant antibodies, enriched for specificity and activity against therapeutic targets, as described in PCT/US2020/030878 filed on Apr. 30, 2020; PCT/US2021/037232 filed on Jun. 14, 2021; and PCT/US2021/044523 filed on Aug. 4, 2021, which are incorporated by reference in their entireties.

The general strategy was adopted, modified and further improved to generate rHBIG (recombinant anti-HBV hyperimmune gammaglobulin) that will enable HBsAg clearance.

In one aspect, the present disclosure provides rHBIG, i.e., a library of novel ABPs (novel antigen-binding proteins, also called recombinant polyclonal antibody proteins, recombinant hyperimmune globulins, or recombinant hyperimmunes) with binding specificity to HBV antigens, e.g., HBV surface antigen, and methods of using such recombinant polyclonal antibodies, e.g., as human therapeutics. The polyclonal therapy can ensure retained targeting of the many HBV genotypes present and the large number of escape mutant variants that arise.

In some embodiments, the polyclonal therapy can target any one of HBV genotypes A to I. In some embodiments, the polyclonal therapy can target HBV genotype A. In some embodiments, the polyclonal therapy can target HBV genotype B. In some embodiments, the polyclonal therapy can target HBV genotype C. In some embodiments, the polyclonal therapy can target HBV genotype D. In some embodiments, the polyclonal therapy can target HBV genotype E. In some embodiments, the polyclonal therapy can target HBV genotype F. In some embodiments, the polyclonal therapy can target HBV genotype G. In some embodiments, the polyclonal therapy can target HBV genotype H. In some embodiments, the polyclonal therapy can target HBV genotype I. In some embodiments, the polyclonal therapy can target HBV genotypes A to D. In some embodiments, the polyclonal therapy can target any one of HBV genotypes A to D.

The recombinant polyclonal antibodies are recombinant, and their sequences are derived from peripheral blood plasma cells or plasmablasts. The peripheral blood plasma cells or plasmablasts of a donor exposed to an HBV antigen by HBV infection or vaccination are mobilized, and the peripheral blood B cells, plasma cells or plasmablasts are specifically separated from other peripheral blood cells. The peripheral blood cells can come from any mammal, for example a mouse, a rat, a human, a monkey, a horse, or a cow.

The present disclosure further provides host cells for production of the novel recombinant polyclonal antibodies. The host cells can produce more than 100, 1000, or more antibodies with binding specificity to HBV antigens, e.g., HBV surface antigen. The recombinant polyclonal antibodies can be isolated and purified from the supernatant of the host cell culture.

Accordingly, the present disclosure provides a pharmaceutical composition comprising: at least 100 unique human recombinant antibodies or antigen-binding proteins (ABPs) specifically binding to a Hepatitis B virus (HBV) surface antigen; and a pharmaceutically acceptable excipient, wherein each of the recombinant antibodies (e.g., ABPs) comprises a cognate pair of heavy chain and light chain variable regions from a single cell out of a blood sample from a donor exposed to the HBV surface antigen; and has been generated from a polynucleotide construct encoding the cognate pair of heavy chain and light chain variable regions.

In some embodiments, each of the ABP has been generated by the process of a. isolating single cells from the blood sample from the donor vaccinated with an HBV vaccine; b. amplifying polynucleotides, wherein each of the polynucleotides encodes a cognate pair of heavy chain and light chain variable regions from one of the single cells by overlap extension reverse transcriptase polymerase chain reaction (OE-RT-PCR); c. cloning the polynucleotides obtained from the amplification into an expression vector, thereby obtaining the constructs encoding antibody fragments; d. generating antibody expression constructs using the constructs or a subset of the constructs, wherein each of the antibody expression constructs encodes a light chain variable region, a kappa or lambda-type light chain constant region, a heavy chain variable region, and a heavy chain constant region, e. introducing the antibody expression constructs into a cell line, and f. expressing antibodies from the antibody expression constructs in the cell line.

In some embodiments, the donor has been selected as having an anti-HBV antigen serum titer of 50 IU/mL or higher. In some embodiments, the donor has been selected as having an anti-HBV antigen serum titer of 100 IU/mL or higher, 150 IU/mL or higher, or 200 IU/mL or higher.

In some embodiments, the blood sample comprises CD27+ cells purified from PBMC of the donor. In some embodiments, the blood sample comprises CD43+ cells purified from PBMC of the donor. In some embodiments, the the blood sample comprises CD27+ cells and CD43+ cells purified from PBMC of the donor. In some embodiments, the blood sample comprises CD27+ or CD43+ cells expressing antibodies comprising a IgK light chain. In some embodiments, the the blood sample comprises CD27+ or CD43+ cells expressing antibodies comprising a IgL light chain. In some embodiments, the process further comprises preparing the blood sample by isolating CD27+ or CD43+ cells from PBMC of the donor.

In some embodiments, the process further comprises preparing the blood sample by isolating CD27+ or CD43+ cells expressing antibodies comprising a IgK light chain from PBMC of the donor. In some embodiments, the process further comprises preparing the blood sample by isolating CD27+ or CD43+ cells expressing antibodies comprising a IgL light chain from PBMC of the donor. In some embodiments, the process further comprises preparing the blood sample by isolating CD27+ cells and CD43+ cells from PBMC of the donor. In some embodiments, the process further comprises preparing the blood sample by isolating CD27+ cells and CD43+ cells expressing antibodies comprising a IgK light chain from PBMC of the donor. In some embodiments, the process further comprises preparing the blood sample by isolating CD27+ cells and CD43+ cells expressing antibodies comprising a IgL light chain from PBMC of the donor.

In some embodiments, the donor has been vaccinated with a booster dose of the HBV vaccine. In some embodiments, the blood sample was obtained from the donor on day 7-15 after the booster vaccination. In some embodiments, the blood sample was obtained from the donor on day 10-12 after the booster vaccination. In some embodiments, the library comprises ABPs originated from two or more donors. In some embodiments, the library comprises ABPs originated from two, three, four, five, six, seven, eight, nine, ten, eleven, twelve, thirteen, fourteen, fifteen, sixteen, seventeen, eighteen, nineteen, or twenty donors. In some embodiments, the library comprises ABPs originated from at least five, at least ten, at least fifteen, at least twenty, at least twenty-five, or at least thirty donors.

In some embodiments, the step a. comprises isolating single cells from blood samples from two or more donors vaccinated with an HBV vaccine. In some embodiments, the step a. comprises isolating at least 100 single cells. In some embodiments, step a. comprises isolating single cells from blood samples from two or more donors vaccinated with the HBV vaccine. In some embodiments, the step a. comprises isolating single cells from blood samples from at least five, at least ten, at least fifteen, at least twenty, at least twenty-five, or at least thirty donors.

In some embodiments, the process further comprises the step of obtaining the subset of the constructs after step c. by expressing the antibody fragments from the constructs and enriching the subset of the constructs based on the binding activities of the antibody fragments against the HBV surface antigen. In some embodiments, the library comprises at least 1000 or at least 10,000 ABPs.

In some embodiments, each of the ABP is an IgG1 antibody. In some embodiments, each of the ABP comprises a IgK light chain. In some embodiments, the HBV vaccine is Engerix-B HBV vaccine. In some embodiments, the at least 100 ABPs have a binding EC50 against the HBV surface antigen lower than 1 μg/ml. In some embodiments, the at least 100 ABPs have a binding EC50 against the HBV surface antigen lower than 0.5 μg/ml, 0.1 μg/ml, 0.05 μg/ml, 0.01 μg/ml, or 0.001 μg/ml. In some embodiments, the at least 100 recombinant antibodies have EC50 against the HBV surface antigen lower than 1 μg/ml. In some embodiments, the at least 100 recombinant antibodies have EC50 against the HBV surface antigen lower than 0.5 μg/ml, 0.1 μg/ml, 0.05 μg/ml, 0.01 μg/ml, or 0.001 μg/ml.

In some embodiments, the at least 100 ABPs have at least 100-fold lower binding EC50 against the HBV surface antigen compared to a plasma sample from the donor vaccinated with an HBV vaccine.

In some embodiments, the at least 100 ABPs have at least 100-fold lower EC50 against the HBV surface antigen compared to a plasma sample from the donor exposed to the HBV surface antigen. In some embodiments, the at least 100 ABPs or antigen-binding proteins (ABPs) have at least 500-fold, 1,000-fold, 1,500-fold, 2,000-fold, 3,000-fold, 4,000-fold, or 5,000-fold lower EC50 against the HBV surface antigen compared to a plasma sample from the donor exposed to the HBV surface antigen.

In some embodiments, wherein the EC50 was measured by ELISA.

In some embodiments, the at least 100 ABPs have at least 100-fold lower IC50 against HBV compared to a plasma sample from the donor exposed to the HBV surface antigen. In some embodiments, the at least 100 ABPs have at least 500-fold 1,000-fold, 2,000-fold, 3,000-fold, 4,000-fold, 5,000-fold, 10,000-fold, 20,000-fold, 30,000-fold, 40,000-fold or 50,000-fold lower IC50 against HBV compared to a plasma sample from the donor exposed to the HBV surface antigen.

In some embodiments, the IC50 against HBV was measured by detecting neutralization of live HBV infection to human hepatocyte cells.

In some embodiments, the pharmaceutical composition comprises at least 500, 1,000, 2,000, 3,000, 4,000, 5,000, 10,000, 100,000, or more unique human ABPs specifically binding to an HBV surface antigen. In some embodiments, each of the recombinant antibodies is an IgG antibody. In some embodiments, each of the ABPs is a human IgG1 subtype.

In some embodiments, the polynucleotide construct further encodes a kappa or lambda-type light chain constant region, and a heavy chain IgG constant region. In some embodiments, the donor has been exposed to HBV. In some embodiments, the donor has been vaccinated against the HBV surface antigen.

In some embodiments, the polynucleotide construct has been enriched based on binding affinity of a protein encoded by the polynucleotide construct to the HBV surface antigen. In some embodiments, the polynucleotide construct has been enriched based on neutralizing activity of a protein encoded by the polynucleotide construct to HBV. In some embodiments, the neutralizing activity is measured by detecting neutralization of live HBV infection to human hepatocyte cells.

In some embodiments, the at least 100 ABPs have a neutralization IC50 against HBV lower than 0.5 μg/ml.

In some embodiments, the at least 100 ABPs have a neutralization IC50 against HBV lower than 0.1 μg/ml, 0.05 μg/ml, 0.01 μg/ml, 0.005 μg/ml, 0.001 μg/ml, 20 ng/ml, or 2.8 ng/ml. In some embodiments, the at least 100 ABPs have at least 100-fold lower neutralization IC50 against HBV compared to a plasma sample from the donor vaccinated with an HBV vaccine.

In some embodiments, the at least 100 ABPs have at least 500-fold 1,000-fold, 2,000-fold, 3,000-fold, 4,000-fold, 5,000-fold, 10,000-fold, 20,000-fold, 30,000-fold, 40,000-fold, or 50,000-fold lower IC50 against HBV compared to a plasma sample from the donor exposed to the HBV surface antigen. In some embodiments, the IC50 against HBV was measured by detecting neutralization of live HBV infection to human hepatocyte cells.

In some embodiments, method comprises at least 500, 1,000, 2,000, 3,000, 4,000, 5,000, 10,000, 100,000, or more unique human ABPs specifically binding to an HBV surface antigen. In some embodiments, the polynucleotide construct has been enriched based on binding affinity of a protein encoded by the polynucleotide construct to the HBV surface antigen.

In some embodiments, the polynucleotide construct has been enriched based on binding affinity of a protein encoded by the polynucleotide construct to a first HBV surface antigen and a second HBV surface antigen. In some embodiments, the first antigen and the second antigen are independently selected from adw, adr, ayw, and ayr antigens.

In some embodiments, the composition further comprises a compound selected from benzalkonium chloride, benzyl alcohol, benzoic acid, salicylic acid, thimerosal, phenethyl alcohol, methylparaben, propylparaben, chlorhexidine, sorbic acid, and hydrogen peroxide in an amount effective to preserve the pharmaceutical composition.

In some embodiments, the composition is free of preservatives.

In some embodiments, the at least 100 ABPs at 10 g/L to 300 g/L concentration.

In some embodiments, the at least 100 ABPs at 10 g/L to 50 g/L concentration, at 10 g/L to 100 g/L concentration, or at 10 g/L to 200 g/L concentration.

In some embodiments, the at least 100 ABPs at 10, 20, 30, 40, or 50 g/L concentration.

In some embodiments, further comprising 20 mM acetate, 270 mM sucrose, and 0.2 mg/mL (0.02% w/v) Polysorbate 20, pH 4.8.

In some embodiments, the composition is free of alive or dead HBV or any protein or polynucleotide produced from HBV.

In some embodiments, the composition has a CEX-HPLC peak distribution having at least 80%, 90%, 95%, 98%, or 99% overlap with the distribution in FIG. 20 in terms of peak sizes, peak retention times, or peak numbers, wherein the CEX-HPLC is performed with a pH gradient from a low pH (pH 5-6) mobile phase A to a high pH (pH 10-11) mobile phase B.

In some embodiments, the composition has a CEX-HPLC peak distribution having at least 80%, 90%, 95%, 98%, or 99% overlap with the distribution in FIG. 20 in terms of peak sizes, peak retention times, and peak numbers, wherein the CEX-HPLC is performed with a pH gradient from a low pH (pH 5-6) mobile phase A to a high pH (pH 10-111) mobile phase B.

In some embodiments, the composition has a CEX-HPLC peak distribution with a retention time ranging from 10-55 minutes.

In some embodiments, the composition has a CEX-HPLC peak distribution with a retention time ranging from 20-30 minutes.

In some embodiments, the pharmaceutical composition further comprises a compound selected from benzalkonium chloride, benzyl alcohol, benzoic acid, salicylic acid, thimerosal, phenethyl alcohol, methylparaben, propylparaben, chlorhexidine, sorbic acid, and hydrogen peroxide in an amount effective to preserve the pharmaceutical composition.

In some embodiments, the composition is free of preservatives.

In some embodiments, the composition is free of alive or dead HBV or any protein or polynucleotide produced from HBV.

The present disclosure also provides a method of treating a patient exposed to HBV by administering an effective dose of the pharmaceutical composition disclosed herein.

In some embodiments, the patient had exposure to HBV by parenteral exposure, direct mucous membrane contact, or oral ingestion involving HBV surface antigen-positive materials such as blood, plasma, or serum.

In some embodiments, the patient is an infant born to a HBV surface antigen-positive mother. In some embodiments, the patient has HBV exposure by sexual exposure to a HBV surface antigen-positive person or household exposure to a person with acute HBV infection. In some embodiments, the patient has chronic HBV infection.

In some embodiments, the method further comprises the step of immunizing the patient with a HBV surface antigen. In some embodiments, the pharmaceutical composition is administered by intramuscular injection, subcutaneous injection, intravenous injection, or intradermal injection.

In some embodiments, the pharmaceutical composition is administered less than 1 day, 2 days, 3 days, 4 days, 5 days, 6 days or 7 days after the exposure to HBV. In some embodiments, the pharmaceutical composition is administered less than 2 weeks, 3 weeks, 4 weeks, 5 weeks, 6 weeks, 7 weeks, or 8 weeks after the exposure to HBV. In some embodiments, the patient has chronic hepatitis B infection.

In some embodiments, the pharmaceutical composition is administered at least twice, at least three times, at least four times, or more.

In one aspect, the present disclosure provides a method of making recombinant antibodies for treatment of a patient exposed to HBV, comprising: a. isolating single cells from a blood sample from a donor exposed to a Hepatitis B virus (HBV) surface antigen; b. amplifying polynucleotides from the isolated single cells, wherein each polynucleotide encodes a cognate pair of heavy chain and light chain variable regions from a single cell by overlap extension reverse transcriptase polymerase chain reaction (OE-RT-PCR); c. cloning the polynucleotides obtained from the amplification into an expression vector, thereby obtaining polynucleotide constructs encoding the recombinant antibodies; d. generating recombinant antibodies from the polynucleotide constructs; and e. purifying the recombinant antibodies.

In some embodiments, the process further comprises enriching the recombinant antibodies based on binding affinity of the recombinant antibodies to the HBV surface antigen. In some embodiments, the process further comprises enriching the recombinant antibodies based on neutralizing activity of the recombinant antibodies to live HBV.

In another aspect, the present disclosure provides a plurality of recombinant antibodies generated by the method disclosed herein.

The present disclosure also provides a plurality of isolated polynucleotides, wherein each of the isolated polynucleotides encodes one of the plurality of recombinant antibodies disclosed herein. The present disclosure further provides a plurality of polynucleotide constructs, comprising the isolated polynucleotides disclosed herein cloned into an expression vector.

One aspect of the present disclosure relates to a plurality of host cells comprising the plurality of isolated polynucleotides or the plurality of polynucleotide constructs of disclosed herein. In some embodiments, the host cells are CHO cells.

In some embodiments, the composition has a CEX-HPLC peak distribution with a retention time ranging from 22-28 minutes.

In some embodiments, the composition has a CEX-HPLC peak distribution with a retention time of at least 10 minutes, at least 15 minutes, at least 20 minutes, at least 25 minutes, at least 30 minutes, at least 35 minutes, at least 40 minutes, at least 45 minutes, at least 50 minutes, or at least 55 minutes.

Another aspect of the present disclosure includes a method of making antigen-binding proteins (ABPs) for treatment of a patient exposed to HBV, comprising: a. isolating single cells from a blood sample from a donor previously vaccinated with a Hepatitis B virus (HBV) vaccine; b. amplifying polynucleotides from the isolated single cells, wherein each polynucleotide encodes a cognate pair of heavy chain and light chain variable regions from a single cell by overlap extension reverse transcriptase polymerase chain reaction (OE-RT-PCR); c. cloning the polynucleotides obtained from the amplification into an expression vector, thereby obtaining polynucleotide constructs encoding recombinant antibodies; d. introducing the antibody expression constructs into a cell line; e. expressing antibodies from the antibody expression constructs in the cell line; and f. obtaining the ABPs.

In some embodiments, the method further comprises, culturing the cell line and purifying the antibodies from the cell line.

In some embodiments, purifying comprises performing at least one of the following steps on the antibodies: i) affinity chromatography, ii) low pH virus inactivation, iii) hydrophobic interaction chromatography, or membrane filtration, iv) multimodal anion exchange chromatography or membrane filtration, (v) multimodal cation exchange chromatography, (vi) anion exchange chromatography or membrane filtration, (vii) cation exchange chromatography, (viii) virus filtration, and ix) ultrafiltration and/or diafiltration.

In some embodiments, the purifying step is performed by two, three, four, five, six, seven, eight, or all nine of the following steps: (i) affinity chromatography, (ii) low pH virus inactivation, (iii) hydrophobic interaction chromatography or membrane filtration, (iv) multimodal anion exchange chromatography or membrane filtration, (v) multimodal cation exchange chromatography, (vi) anion exchange chromatography or membrane filtration, (vii) cation exchange chromatography, (viii) virus filtration, and (ix) ultrafiltration and/or diafiltration.

In some embodiments, the donor has been selected as having an anti-HBV antigen serum titer of 50 IU/mL or higher. In some embodiments, the donor has been selected as having an anti-HBV antigen serum titer of 100 IU/mL or higher, 150 IU/mL or higher, or 200 IU/mL or higher. In some embodiments, the blood sample comprises CD27+ cells or CD43+ cells purified from PBMC of the donor. In some embodiments, the blood sample comprises CD27+ cells and CD43+ cells purified from PBMC of the donor. In some embodiments, the blood sample comprises CD27+ or CD43+ cells expressing antibodies comprising a IgK light chain. In some embodiments, the method comprises preparing the blood sample by isolating CD27+ or CD43+ cells from PBMC of the donor. In some embodiments, the method comprises preparing the blood sample by isolating CD27+ or CD43+ cells expressing antibodies comprising a IgK light chain from PBMC of the donor. In some embodiments, the method comprises preparing the blood sample by isolating CD27+ or CD43+ cells expressing antibodies comprising a IgL light chain from PBMC of the donor. In some embodiments, the donor has been vaccinated with a booster dose of the HBV vaccine. In some embodiments, the blood sample was obtained from the donor on day 7-15 after the booster vaccination. In some embodiments, the blood sample was obtained from the donor on day 10-12 after the booster vaccination. In some embodiments, step a. comprises isolating single cells from blood samples obtained from two or more donors. In some embodiments, step a. comprises isolating single cells from blood samples obtained from two, three, four, five, six, seven, eight, nine, ten, eleven, twelve, thirteen, fourteen, fifteen, sixteen, seventeen, eighteen, nineteen, or twenty donors. In some embodiments, step a. comprises isolating single cells from blood samples obtained from at least five, at least ten, at least fifteen, at least twenty, at least twenty-five, or at least thirty donors.

In some embodiments, the method further comprises enriching the recombinant antibodies based on binding affinity of the recombinant antibodies to an HBV surface antigen. In some embodiments, the enriching step comprises enriching the recombinant antibodies based on binding affinity of the recombinant antibodies to at least two different antigens of HBV.

Another aspect of the present disclosure comprises a plurality of ABPs generated by the method of the present disclosure.

Another aspect of the present disclosure comprises a plurality of isolated polynucleotides, wherein each of the isolated polynucleotides encodes one of the plurality of ABPs disclosed herein

Another aspect of the present disclosure comprises a plurality of polynucleotide constructs, comprising the isolated polynucleotides disclosed herein cloned into an expression vector.

Another aspect of the present disclosure includes a plurality of host cells comprising the plurality of isolated polynucleotides disclosed herein or the plurality of polynucleotide constructs disclosed herein. In some embodiments, the host cells are CHO cells.

Another aspect of the present disclosure includes a pharmaceutical composition comprising the plurality of ABPs disclosed herein.

Another aspect of the present disclosure includes method of treating or preventing viral infection in a patient, comprising administering an effective amount of the pharmaceutical composition disclosed herein.

In some embodiments, the patient had or have a risk of exposure to Hepatitis B virus (HBV) by parenteral exposure, direct mucous membrane contact, or oral ingestion involving HBV surface antigen-positive materials such as blood, plasma, or serum.

In some embodiments, the patient is an infant born or will be born to an Hepatitis B virus (HBV) surface antigen-positive mother.

In some embodiments, the patient: has one or more of Hepatitis B virus (HBV) exposure by sexual exposure to an HBV surface antigen-positive person; household exposure to a person with acute HBV infection; chronic HBV infection; or chronic hepatitis B infection. In some embodiments, the patient: has HBV exposure or has a risk of HBV exposure to at least one HBV mutant by sexual exposure to a HBV mutant surface antigen-positive person, household exposure to a person with acute HBV infection; or is an infant born to an HBV mutant surface antigen-positive mother.

In some embodiments, the at least one HBV mutant comprises an HBV surface antigen with a mutation selected from: P120Q, T126S, Q129H, M133D, M133T, F134S, K141E, P142L, P142S, D144A, D144N, and G145R.

In some embodiments, the at least one HBV mutant comprises at least two HBV mutants, wherein the at least two mutants comprise an HBV surface antigen with mutations G145R and P142L.

In some embodiments, the patient has chronic HBV infection, optionally wherein the patient has one or more of HBeAg negative chronic HBV infection for more than 6 months, HBsAg in serum for more than 6 months, HBsAg concentration in serum between ≥100 IU/mL and ≤2000 IU/mL at screening, received stable dose of NAs for over 6 months, and/or serum HBV DNA concentration not higher than 50 IU/mL at screening.

In some embodiments, the method further comprises the step of immunizing the patient with an HBV surface antigen.

In some embodiments, the pharmaceutical composition is administered intramuscularly, subcutaneously, intravenously, or intradermally. In some embodiments, the pharmaceutical composition is administered less than 1 day, 2 days, 3 days, 4 days, 5 days, 6 days, or 7 days after the exposure to HBV. In some embodiments, the pharmaceutical composition is administered less than 2 weeks, 3 weeks, 4 weeks, 5 weeks, 6 weeks, 7 weeks, or 8 weeks after the exposure to HBV. In some embodiments, the effective amount comprises a single dose of 300 mg/kg or less of the ABPs. In some embodiments, the effective amount comprises a single dose of 100 mg/kg or less of the ABPs. In some embodiments, the effective amount comprises a single dose of 30 mg/kg or less of the ABPs. In some embodiments, the effective amount comprises a single dose of 1-100 mg/kg of the ABPs.

In some embodiments, the effective amount comprises a single dose of 10 mg, 20 mg, 21 mg, 70 mg, 210 mg, 700 mg, 1 g, 1.5 g, 2 g, 3 g, 4 g, 5 g, 6 g, 7 g, 8 g, 9 g, or more of the ABPs.

In some embodiments, the effective amount comprises a single dose of 20-30 mg/kg, 10-20 mg/kg, 1-10 mg/kg or 1-5 mg/kg of the ABPs. In some embodiments, the effective amount is an amount that leads to the serum concentration of the at least 100 ABPs in the range of at least 5,000 ng/mL, at least 10,000 ng/mL, at least 20,000 ng/mL, at least 30,000 ng/mL, at least 40,000 ng/mL or at least 50,000 ng/mL. In some embodiments, the pharmaceutical composition is administered more than once.

In some embodiments, the administration is repeated every week, every two weeks, every 3 weeks, every 4 weeks, every 5 weeks, every 6 weeks, every 7 weeks, or every 8 weeks. In some embodiments, the administration is repeated every one month, every two months, every three months, every four months, every five months, or every six months.

In some embodiments, the repeated administration is performed for 3 months, 4 months, 5 months, 6 months, 7 months, 8 months, 9 months, 10 months, 11 months, 12 months, or more. In some embodiments, the patient has been exposed to or has a risk of exposure to at least one HBV genotype selected from: HBV genotype A, HBV genotype B, HBV genotype C, HBV genotype D, HBV genotype E, HBV genotype F, HBV genotype G, HBV genotype H, and HBV genotype I. In some embodiments, the patient has been exposed to or has a risk of exposure to HBV genotype A, B, C, or D. In some embodiments, the ABPs comprise a greater binding potency compared to HyperHEP B and/or anti-HBV monoclonal antibodies. In some embodiments, the ABPs have at least a 1,000-fold stronger binding potency compared to HyperHEP B. In some embodiments, the ABPs comprise a greater affinity to at least one HBV mutant with a surface antigen comprising a mutation selected from: P120Q, T126S, Q129H, M133D, M133T, F134S, K141E, P142L, P142S, D144A, D144N, and G145R, compared to HyperHEP B and/or anti-HBV monoclonal antibodies.

Another aspect of the present disclosure includes a drug product comprising the pharmaceutical composition of the present disclosure.

Another aspect of the present disclosure is a unit dose of the pharmaceutical composition described herein.

### 6. BRIEF DESCRIPTION OF THE DRAWINGS

FIG. 1 shows results from ELISA binding assay of rHBIG generation with various blood samples (CD27+ high IgK cells; CD27+ high IgL cells; CD27+ low IgK cells; CD43+ high IgK cells; CD43+ high IgL cells; and CD43+ low IgK cells).

FIG. 2 provides Jaccard and Morisita indices calculated for two sets of two RHBIG-1 Bioreactor Runs.

FIG. 3 shows results from HBsAg binding ELISA of RHBIG-1 compared to HyperHEP B as Reference.

FIG. 4 shows HBV neutralization by RHBIG-1 offers prophylactic prevention of in vitro hepatocyte infection.

FIG. 5 shows correlation between HBsAg binding ELISA and In Vitro Neutralization assay results.

FIG. 6 shows binding capacity of RHBIG-1 to mutation variants of HBsAg.

FIG. 7 shows prophylactic protection from HBV infection by RHBIG-1 in vivo.

FIG. 8 shows antibody products HyperHEP B (open circle), mAb 1 (open square), mAb 2 (triangle), and RHBIG-1 GMP DS (nabla)) were tested by ELISA to determine potency. Test articles were serially diluted 1:3. Starting concentrations were selected to provide a full binding curve to determine calculation of EC50 (half maximal binding concentration) by 4 point logistic regression and graphed (GraphPad, Prism).

FIG. 9 shows RHBIG-1 (red), HyperHEP B (blue), HBV mAb 1 (light greens), mAb 2 (dark green), and IVIG (black) were tested in triplicate for neutralizing potency against 1,000 MOI of HBV and calculated relative to a no antibody infection control. A 3 point logistic regression curve was used to calculate IC50 (half maximal inhibitory concentration values), curves represent mean and standard deviation (GraphPad, Prism).

FIG. 10 shows clinically relevant mutations to the “a” determinant region of HBsAg were generated and used as plate coats for ELISA assays to determine binding capacity of RHBIG-1 (red) and mAb 1 (green). Plots show the optical density measurement of HRP signal, indicating the level of test article bound to the mutation plate coat protein. Abbreviations: OD450 nm=optical density measurement at 450 nanometers.

FIG. 11 shows pan genotype neutralization by RHBIG-1 of HDV pseudo expressed HBV genotypes A-H. Percentage of dHepaRG cells infected by HDV-1 expressing HBsAg from each indicated HBV genotype A, B, C, D, E, F, G, and H, when 100 MOI of active virus was preincubated with indicated titrations of antibody prior to exposure to HepaRG cells and cultured for 6 days (circle=RHBIG-1; triangle=mAb 1; square=HyperHEP B). Determination of infection was based on detection of intracellular HDV antigen by immunofluorescence.

FIG. 12 shows RHBIG-1 induces ADCC signal pathway in vitro. Jurkat T cells were modified to express human Fc receptor gamma IIIa (FcgRIIIa) with downstream signaling pathways linked to the expression of luciferase, induction of FcR signaling produces luciferase that can be quantitatively measured compared to controls. The induction of antibody dependent cellular cytotoxicity signaling above baseline (dotted line) is shown for indicated concentrations of test article (RHBIG-1 (open square); HyperHEP B (diamond), anti-HBsAg mAb (open circle)). Error bars represent the standard deviation. Data is representative of 3 separate experiments.

FIG. 13 shows RHBIG-1 induces ADCP signal pathway in vitro. Jurkat T cells were modified to express human Fc receptor gamma IIa (FcγRIIa) with downstream signaling pathways linked to the expression of luciferase, induction of FcR signaling produces luciferase that can be quantitatively measured compared to controls. The induction of antibody dependent cellular phagocytosis signaling above baseline (dotted line) is shown for indicated concentrations of test article (RHBIG-1 in red; HyperHEP B in blue; anti-HBsAg mAb in green). Error bars represent the standard deviation. Data representative of 3 separate experiments.

FIG. 14 shows whole blood peripheral human immune cells binding to RHBIG-1 in complex with HBsAg via Fc receptor driven interaction (RHBIG-1_VIT_007). Percent of each given immune cell population with detection of positive PE-Cy7+ signal for immune complex; RHBIG-1+HBsAg (dark red) or RHBIG-1 only (red).

FIG. 15 shows dose-dependent reduction of HBsAg and HBV-DNA by RHBIG-1. FIG. 15A) Change in serum HBsAg level in AAV/HBV infected C57BL/6J mice (n=6 per group) up to study end 28 days after test article dosing compared to average baseline level for each group. RHBIG-1 at two dose levels (light and dark red) was compared to HyperHEP B (blue), mAb 1 (green), and PBS control (black). FIG. 15B) Change in serum HBV-DNA level in AAV/HBV infected mice (n=6 per group) days after test article dosing compared to baseline level. Error bars represent standard error of the mean for each group. Significant changes are calculated based on each intra-mouse change from day 0 baseline to the indicated time after dosing using a paired Students t Test (* p<0.05; ** p<0.01; *** p<0.001; **** p<0.0001); significant changes above baseline after day 14 are not indicated.

FIG. 16A-16B shows RHBIG-1 dose dependent depletion of HBsAg in male and female mice. Change in serum HBsAg level in AAV/HBV infected C57BL/6J male (top) and female (bottom) mice up to study end at 14 days after test article dosing compared to average baseline level for each group taken at day 0 (n=5/time point/group). RHBIG-1 at two dose levels (10 mg/kg in light red and 100 mg/kg in dark red) was compared to HyperHEP B (100 mg/kg, in blue) and PBS control (black). Error bars represent standard error of the mean for each group. Significant changes are calculated based on each intra-mouse change from day 0 baseline to the indicated time after dosing using a paired Students t Test (** p<0.01; *** p<0.001; **** p<0.0001). Abbreviations: HBsAg; Hepatitis B virus surface antigen. LFC; log fold change.

FIGS. 17A-17C show serum HBsAg titer temporally after RHBIG-1 dosing (FIGS. 17A, 17B) Change in serum HBsAg level in AAV/HBV infected C57BL/6J mice up to 35 days after test article dosing for RHBIG-1 at 10 mg/kg (FIG. 17A) and 100 mg/kg (FIG. 17B). Grey coloration indicates data points from mice in subgroup A and black indicate data from subgroup B. Baseline HBsAg from subgroups A and B are combined for the day 0 baseline. Red bars indicate the median value of each time point. (FIG. 17C) The level of HBsAg temporally after test article delivery linking data from subgroups A and B for RHBIG-1 delivered at 10 mg/kg (light red) and 100 mg/kg (dark red). The left part of the X axis is measured in hours, the right part of the X axis is annotated with day equivalent for the number of hours elapsed from dosing. Error bars represent standard error of the mean for each group. Abbreviations: HBsAg=hepatitis B virus surface antigen. LLOQ=lower limit of quantification. MPK=mg/kg.

FIGS. 18A-18C shows unbound RHBIG-1 after IV dosing in an antigenic system (FIG. 18A, 18B) Change in detectable RHBIG-1 level in AAV/HBV infected C57BL/6J mice serum up to 35 days after dosing for RHBIG-1 at 10 mg/kg (FIG. 18A) and 100 mg/kg (FIG. 18B). Grey coloration indicates data points from mice in subgroup A and black coloration indicates data from subgroup B. Red bars indicate the median value of each time point. (FIG. 18C) The level of RHBIG-1 detected temporally after test article delivery linking data from subgroups A and B for RHBIG-1 delivered at 10 mg/kg (light red) and 100 mg/kg (dark red). The left part of the X axis is measured in hours, the right part of the X axis is annotated with day equivalent for the number of hours elapsed from dosing. Error bars represent standard error of the mean for each group. Abbreviations: HBsAg=hepatitis B virus surface antigen. LLOQ=lower limit of quantification. MPK=mg/kg.

FIG. 19 shows an overlay of mean (+SD) RHBIG-1 Plasma Concentration and HBsAg versus Time by Treatment in AAV/HBV Infected Mice. The graph shows that maximal efficacy was observed at a rHBIG-1 serum concentration of approximately 20,000 ng/mL. Therefore, rHBIG-1 serum concentration of approximately 20,000 ng/mL are required to suppress HBsAg.

FIG. 20 shows CEX-HPLC chromatograms of rHBIG-1 from three separate runs.

FIG. 21 provides CEX-HPLC chromatograms of anti-HBV plasma hyperimmune (HyperHEP), rHBIG-1, a library of ABPs with binding specificity to CoV antigen (rCIG), or a recombinant monoclonal antibody (anti-CTLA-4).

FIG. 22 provides CEX-HPLC chromatograms of rHBIG-1 and six individual antibodies in rHBIG-1 (PN-6103.02, 6104.02, 6105.02, 6115.02, 6116.02, 6117.02).

### 7. DETAILED DESCRIPTION

### 7.1. Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are those well-known and commonly used in the art. Standard techniques can be used for chemical syntheses, chemical analyses, pharmaceutical preparation, formulation, and delivery, and treatment of patients.

The following terms, unless otherwise indicated, shall be understood to have the following meanings:

The term “antibody” is used herein in its broadest sense and includes certain types of immunoglobulin molecules comprising one or more antigen-binding domains that specifically bind to an antigen or epitope. An antibody specifically includes intact antibodies (e.g., intact immunoglobulins), antibody fragments, and multi-specific antibodies. One example of an antigen-binding domain is an antigen-binding domain formed by a V_{H}-V_{L} dimer.

The term “recombinant polyclonal antibody” refers to more than one recombinant antibodies, collectively comprising more than one antigen-binding domains that specifically bind to an antigen or epitope, or multiple antigens and epitopes. The recombinant polyclonal antibodies can be intact antibodies or variants or derivatives thereof. In some embodiments, the antigen-binding domains bind an antigen or epitope with specificity and affinity similar to that of a naturally occurring antibody. In some embodiments, a recombinant polyclonal antibody is a mixture of antibodies. In some embodiments, a recombinant polyclonal antibody comprises scFvs. In some embodiments, a recombinant polyclonal antibody comprises an alternative scaffold. In some embodiments, a recombinant polyclonal antibody consists of alternative scaffolds. In some embodiments, a recombinant polyclonal antibody consists essentially of alternative scaffolds. In some embodiments, a recombinant polyclonal antibody comprises an antibody fragment. In some embodiments, a recombinant polyclonal antibody consists of antibody fragments. In some embodiments, a recombinant polyclonal antibody consists essentially of antibody fragments.

The term “alternative scaffold” refers to a molecule in which one or more regions may be diversified to produce one or more antigen-binding domains that specifically bind to an antigen or epitope. In some embodiments, the antigen-binding domain binds the antigen or epitope with specificity and affinity similar to that of naturally occurring antibodies. Exemplary alternative scaffolds include those derived from fibronectin (e.g., Adnectins™), the β-sandwich (e.g., iMab), lipocalin (e.g., Anticalins®), EETI-II/AGRP, BPTI/LACI-D1/ITI-D2 (e.g., Kunitz domains), thioredoxin peptide aptamers, protein A (e.g., Affibody®), ankyrin repeats (e.g., DARPins), gamma-B-crystallin/ubiquitin (e.g., Affilins), CTLD3 (e.g., Tetranectins), Fynomers, and (LDLR-A module) (e.g., Avimers). Additional information on alternative scaffolds is provided in Binz et al., Nat. Biotechnol., 2005 23:1257-1268; Skerra, Current Opin. in Biotech., 2007 18:295-304; and Silacci et al., J Biol. Chem., 2014, 289:14392-14398; each of which is incorporated by reference in its entirety. Alternative scaffolds comprise one type of ABP.

The term “antigen-binding domain” means the portion of an antibody that is capable of specifically binding to an antigen or epitope.

The terms “full length antibody,” “intact antibody,” and “whole antibody” are used herein interchangeably to refer to an antibody having a structure substantially similar to a naturally occurring antibody structure and having heavy chains that comprise an Fc region.

The term “immunoglobulin” refers to a class of structurally related proteins, e.g., antibodies, generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, PA. Briefly, each heavy chain typically comprises a heavy chain variable region (V_{H}) and a heavy chain constant region (C_{H}). The heavy chain constant region typically comprises three domains, abbreviated C_{H1}, C_{H2}, and C_{H3}. Each light chain typically comprises a light chain variable region (V_{L}) and a light chain constant region. The light chain constant region typically comprises one domain, abbreviated C_{L}.

The term “Fc region” means the C-terminal region of an immunoglobulin heavy chain that, in naturally occurring antibodies, interacts with Fc receptors and certain proteins of the complement system. The structures of the Fc regions of various immunoglobulins, and the glycosylation sites contained therein, are known in the art. See Schroeder and Cavacini, J. Allergy Clin. Immunol., 2010, 125:S41-52, incorporated by reference in its entirety. The Fc region may be a naturally occurring Fc region, or an Fc region modified as described elsewhere in this disclosure.

The V_{H} and V_{L} regions may be further subdivided into regions of hypervariability (“hypervariable regions (HVRs)” also called “complementarity determining regions” (CDRs)) interspersed with regions that are more conserved. The more conserved regions are called framework regions (FRs). Each V_{H} and V_{L} generally comprises three CDRs and four FRs, arranged in the following order (from N-terminus to C-terminus): FR1-CDR1-FR2-CDR2-FR3-CDR3-FR4. The CDRs are involved in antigen binding, and influence antigen specificity and binding affinity of the antibody. See Kabat et al., Sequences of Proteins of Immunological Interest 5th ed. (1991) Public Health Service, National Institutes of Health, Bethesda, MD, incorporated by reference in its entirety.

The light chain from any vertebrate species can be assigned to one of two types, called kappa (κ) and lambda (λ), based on the sequence of its constant domain.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgAQ1, and IgA2.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Pluckthun, J. Mol. Biol., 2001, 309:657-70 (“AHo” numbering scheme); each of which is incorporated by reference in its entirety.

Table 1 provides the exemplary positions of CDR1-L (CDR1 of V_{L}), CDR2-L (CDR2 of V_{L}), CDR3-L (CDR3 of V_{L}), CDR1-H (CDR1 of V_{H}), CDR2-H (CDR2 of V_{H}), and CDR3-H (CDR3 of V_{H}), as identified by the Kabat and Chothia schemes. For CDR1-H, residue numbering is provided using both the Kabat and Chothia numbering schemes.

CDRs may be assigned, for example, using antibody numbering software, such as Abnum, available at www.bioinf.org.uk/abs/abnum/, and described in Abhinandan and Martin, Immunology, 2008, 45:3832-3839, incorporated by reference in its entirety.

TABLE 1
Residues in CDRs according to Kabat
and Chothia numbering schemes.

| CDR | Kabat | Chothia |
|---|---|---|
| CDR1-L | 24-34 | 24-34 |
| CDR2-L | 50-56 | 50-56 |
| CDR3-L | 89-97 | 89-97 |
| CDR1-H (Kabat Numbering) | 31-35B | 26-32 or 34* |
| CDR1-H (Chothia Numbering) | 31-35 | 26-32 |
| CDR2-H | 50-65 | 52-56 |
| CDR3-H | 95-102 | 95-102 |

*The C-terminus of CDR1-H, when numbered using the Kabat numbering convention, varies between 32 and 34, depending on the length of the CDR.

The “EU numbering scheme” is generally used when referring to a residue in an antibody heavy chain constant region (e.g., as reported in Kabat et al., supra).

An “antibody fragment” comprises a portion of an intact antibody, such as the antigen-binding or variable region of an intact antibody. Antibody fragments include, for example, Fv fragments, Fab fragments, F(ab′)₂ fragments, Fab′ fragments, scFv (sFv) fragments, and scFv-Fc fragments.

“Fv” fragments comprise a non-covalently-linked dimer of one heavy chain variable domain and one light chain variable domain.

“Fab” fragments comprise, in addition to the heavy and light chain variable domains, the constant domain of the light chain and the first constant domain (C_{H1}) of the heavy chain. Fab fragments may be generated, for example, by recombinant methods or by papain digestion of a full-length antibody.

“F(ab′)₂” fragments contain two Fab′ fragments joined, near the hinge region, by disulfide bonds. F(ab′)₂ fragments may be generated, for example, by recombinant methods or by pepsin digestion of an intact antibody. The F(ab′) fragments can be dissociated, for example, by treatment with ß-mercaptoethanol.

“Single-chain Fv” or “sFv” or “scFv” antibody fragments comprise a V_{H} domain and a V_{L} domain in a single polypeptide chain. The V_{H} and V_{L} are generally linked by a peptide linker. See Plückthun A. (1994). In some embodiments, the linker is a (GGGGS)ₙ (SEQ ID NO: 1). In some embodiments, n=1, 2, 3, 4, 5, or 6. See Antibodies from Escherichia coli. In Rosenberg M. & Moore G. P. (Eds.), The Pharmacology of Monoclonal Antibodies vol. 113 (pp. 269-315). Springer-Verlag, New York, incorporated by reference in its entirety.

“scFv-Fc” fragments comprise an scFv attached to an Fc domain. For example, an Fc domain may be attached to the C-terminal of the scFv. The Fc domain may follow the V_{H} or V_{L}, depending on the orientation of the variable domains in the scFv (i.e., V_{H}-V_{L} or V_{L}-V_{H}). Any suitable Fc domain known in the art or described herein may be used. In some cases, the Fc domain comprises an IgG4 Fc domain.

The term “single domain antibody” refers to a molecule in which one variable domain of an antibody specifically binds to an antigen without the presence of the other variable domain. Single domain antibodies, and fragments thereof, are described in Arabi Ghahroudi et al., FEBS Letters, 1998, 414:521-526 and Muyldermans et al., Trends in Biochem. Sci., 2001, 26:230-245, each of which is incorporated by reference in its entirety.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “polyclonal antibody” refers to a mixture of at least two monoclonal antibodies. Polyclonal antibodies may be either monospecific or polyspecific.

The term “chimeric antibody” refers to an antibody in which a portion of the heavy and/or light chain is derived from a particular source or species, while the remainder of the heavy and/or light chain is derived from a different source or species. [00139]“Humanized” forms of non-human antibodies are chimeric antibodies that contain minimal sequence derived from the non-human antibody. A humanized antibody is generally a human antibody (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. Such modifications may be made to further refine antibody function. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

A “human antibody” is one which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

An “isolated antibody” or “isolated nucleic acid” is an antibody or nucleic acid that has been separated and/or recovered from a component of its natural environment. Components of the natural environment may include enzymes, hormones, and other proteinaceous or nonproteinaceous materials. In some embodiments, an isolated antibody is purified to a degree sufficient to obtain at least 15 residues of N-terminal or internal amino acid sequence, for example by use of a spinning cup sequenator. In some embodiments, an isolated antibody is purified to homogeneity by gel electrophoresis (e.g., SDS-PAGE) under reducing or nonreducing conditions, with detection by Coomassie blue or silver stain. An isolated antibody includes an antibody in situ within recombinant cells, since at least one component of the antibody's natural environment is not present. In some aspects, an isolated antibody or isolated nucleic acid is prepared by at least one purincation step. In some embodiments, an isolated antibody or isolated nucleic acid is purified to at least 80%, 85%, 90%, 95%, or 99% by weight. In some embodiments, an isolated antibody or isolated nucleic acid is purified to at least 80%, 85%, 90%, 95%, or 99% by volume. In some embodiments, an isolated antibody or isolated nucleic acid is provided as a solution comprising at least 85%, 90%, 95%, 98%, 99% to 100% antibody or nucleic acid by weight. In some embodiments, an isolated antibody or isolated nucleic acid is provided as a solution comprising at least 85%, 90%, 95%, 98%, 99% to 100% ABP or nucleic acid by volume.

“Affinity” refers to the strength of the sum total of non-covalent interactions between a single binding site of a molecule (e.g., an antibody) and its binding partner (e.g., an antigen or epitope). Unless indicated otherwise, as used herein, “affinity” refers to intrinsic binding affinity, which reflects a 1:1 interaction between members of a binding pair (e.g., antibody and antigen or epitope). The affinity of a molecule X for its partner Y can be represented by the dissociation equilibrium constant (K_{D}). The kinetic components that contribute to the dissociation equilibrium constant are described in more detail below. Affinity can be measured by common methods known in the art, including those described herein. Affinity can be determined, for example, using surface plasmon resonance (SPR) technology (e.g., BIACORE®) or biolayer interferometry (e.g., FORTEBIO®).

With regard to the binding of an antibody to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). Specific binding can be measured, for example, by measuring binding to a target molecule and comparing it to binding to a non-target molecule. Specific binding can also be determined by competition with a control molecule that mimics the epitope recognized on the target molecule. In that case, specific binding is indicated if the binding of the antibody to the target molecule is competitively inhibited by the control molecule.

The term “k_{d}” (sec⁻¹), as used herein, refers to the dissociation rate constant of a particular ABP-antigen interaction. This value is also referred to as the k_{off} value.

The term “kₐ” (M⁻¹×sec⁻¹), as used herein, refers to the association rate constant of a particular ABP-antigen interaction. This value is also referred to as the kₒₙ value.

The term “K_{D}” (M), as used herein, refers to the dissociation equilibrium constant of a particular ABP-antigen interaction. K_{D}=k_{d}/kₐ.

The term “K_{A}” (M⁻¹), as used herein, refers to the association equilibrium constant of a particular ABP-antigen interaction. K_{A}=kₐ/kₐ.

An “immunoconjugate” is an ABP conjugated to one or more heterologous molecule(s).

“Effector functions” refer to those biological activities mediated by the Fc region of an antibody, which activities may vary depending on the antibody isotype. Examples of antibody effector functions include C1q binding to activate complement dependent cytotoxicity (CDC), Fc receptor binding to activate antibody-dependent cellular cytotoxicity (ADCC), and antibody dependent cellular phagocytosis (ADCP).

When used herein in the context of two or more antibodies, the term “competes with” or “cross-competes with” indicates that the two or more antibodies compete for binding to an antigen (e.g., pneumococcus polysaccharide). In one exemplary assay, an antigen is coated on a surface and contacted with a first ABP against the antigen, after which a second ABP against the antigen is added. In another exemplary assay, a first ABP against an antigen is coated on a surface and contacted with the antigen, and then a second ABP against the antigen is added. If the presence of the first ABP against an antigen reduces binding of the second ABP, in either assay, then the antibodies compete. The term “competes with” also includes combinations of antibodies where one ABP reduces binding of another ABP, but where no competition is observed when the antibodies are added in the reverse order. However, in some embodiments, the first and second Antibodies inhibit binding of each other, regardless of the order in which they are added. In some embodiments, one ABP reduces binding of another ABP to its antigen by at least 25%, at least 50%, at least 60%, at least 70%, at least 80%, at least 85%, at least 90%, or at least 95%. A skilled artisan can select the concentrations of the antibodies used in the competition assays based on the affinities of the Antibodies for pneumococcus polysaccharide and the valency of the Antibodies. The assays described in this definition are illustrative, and a skilled artisan can utilize any suitable assay to determine if antibodies compete with each other. Suitable assays are described, for example, in Cox et al., “Immunoassay Methods,” in Assay Guidance Manual [Internet], Updated Dec. 24, 2014 (www.ncbi.nlm.nih.gov/books/NBK92434/; accessed Sep. 29, 2015); Silman et al., Cytometry, 2001, 44:30-37; and Finco et al., J. Pharm. Biomed. Anal., 2011, 54:351-358; each of which is incorporated by reference in its entirety.

The term “epitope” means a portion of an antigen the specifically binds to an ABP or an ABP. Epitopes frequently consist of surface-accessible amino acid residues and/or sugar side chains and may have specific three-dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding, and other amino acid residues, which are not directly involved in the binding. The epitope to which an ABP or an ABP binds can be determined using known techniques for epitope determination such as, for example, testing for ABP or an ABP binding to an antigen.

Percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

A “conservative substitution” or a “conservative amino acid substitution,” refers to the substitution an amino acid with a chemically or functionally similar amino acid. Conservative substitution tables providing similar amino acids are well known in the art. By way of example, the groups of amino acids provided in TABLES 2-4 are, in some embodiments, considered conservative substitutions for one another.

TABLE 2
Selected groups of amino acids that are considered conservative
substitutions for one another, in certain embodiments.

| | |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

TABLE 3
Additional selected groups of amino acids that
are considered conservative substitutions
for one another, in certain embodiments.

| | |
|---|---|
| Group 1 | A, S, and T |
| Group 2 | D and E |
| Group 3 | N and Q |
| Group 4 | R and K |
| Group 5 | I, L, and M |
| Group 6 | F, Y, and W |

TABLE 4
Further selected groups of amino acids that are considered conservative
substitutions for one another, in certain embodiments.

| | |
|---|---|
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions may be found, for example, in Creighton, Proteins: Structures and Molecular Properties 2nd ed. (1993) W. H. Freeman & Co., New York, NY. An ABP generated by making one or more conservative substitutions of amino acid residues in a parent ABP is referred to as a “conservatively modified variant.”

The term “treating” (and variations thereof such as “treat” or “treatment”) refers to clinical intervention in an attempt to alter the natural course of a disease or condition in a subject in need thereof. Treatment can be performed both for prophylaxis and during the course of clinical pathology. Desirable effects of treatment include preventing occurrence or recurrence of disease, alleviation of symptoms, diminish of any direct or indirect pathological consequences of the disease, preventing reinfection or associated symptom, decreasing the rate of disease progression, amelioration or palliation of the disease state, and remission or improved prognosis. Improvements in any conditions can be readily assessed according to standard methods and techniques known in the art. The population of subjects treated by the method of the disease includes subjects suffering from the undesirable condition or disease, as well as subjects at risk for development of the condition or disease.

As used herein, the term “therapeutically effective amount” or “effective amount” refers to an amount of an ABP or pharmaceutical composition provided herein that, when administered to a subject, is effective to produces the desired effect for which it is administered. The exact dose or amount will depend on the purpose of the treatment, and will be ascertainable by one skilled in the art using known techniques (see, e.g., Lloyd (1999) The Art, Science and Technology of Pharmaceutical Compounding). A therapeutically effective amount can be a “prophylactically effective amount” as prophylaxis can be considered therapy. The term “sufficient amount” means an amount sufficient to produce a desired effect.

As used herein, the term “subject” means a mammalian subject. Exemplary subjects include humans, monkeys, dogs, cats, mice, rats, cows, horses, camels, goats, rabbits, and sheep. In certain embodiments, the subject is a human. In some embodiments the subject has a disease or condition that can be treated with an ABP provided herein. In some aspects, the disease or condition is a cancer. In some aspects, the disease or condition is a viral infection.

The term “pharmaceutical composition” refers to a preparation which is in such form as to permit the biological activity of an active ingredient contained therein to be effective in treating a subject, and which contains no additional components which are unacceptably toxic to the subject.

The term “plasma cell” refers to white blood cells that secrete large volumes of antibodies. They are transported by the blood plasma and the lymphatic system. B cells (for example, either germinal center naïve B cells or memory B cells) differentiate into plasma cells that produce antibody molecules closely modelled after the receptors of the precursor B cell. Once released into the blood and lymph, these antibody molecules bind to the target antigen (foreign substance) and initiate its neutralization or destruction. Terminally differentiated plasma cells express relatively few surface antigens, and do not express common pan-B cell markers, such as CD19 and CD20. Instead, plasma cells are identified through flow cytometry by their additional expression of CD138, CD78, and the Interleukin-6 receptor. In humans, CD27 is a good marker for plasma cells, naive B cells are CD27-, memory B-cells are CD27+ and plasma cells are CD27++. The surface antigen CD138 (syndecan-1) is expressed at high levels. Another important surface antigen is CD319 (SLAMF7). This antigen is expressed at high levels on normal human plasma cells. It is also expressed on malignant plasma cells in multiple myeloma. Compared with CD138, which disappears rapidly ex vivo, the expression of CD319 is considerably more stable.

The term “plasmablast” refers to antibody-secreting cells in the peripheral blood, which differentiate from activated B cells, such as memory B cells, upon stimulation with an antigen. The most immature blood cell that is considered of plasma cell lineage is the plasmablast. Plasmablasts secrete more antibodies than B cells, but less than plasma cells. They divide rapidly and are still capable of internalizing antigens and presenting them to T cells. A cell may stay in this state for several days, and then either die or irrevocably differentiate into a mature, fully differentiated plasma cell. Differentiation of mature B cells into plasma cells is dependent upon the transcription factors Blimp-1/PRDM1 and IRF4.

The term “memory B cell” refers to a B cell sub-type that are formed within germinal centers following primary infection and are important in generating an accelerated and more robust antibody-mediated immune response in the case of re-infection (also known as a secondary immune response). Memory B cells do not secrete antibody until activated by their specific antigen.

The term “naïve B cell” refers to a B cell that has not been exposed to an antigen. Once exposed to an antigen, the naïve B cell either becomes a memory B cell or a plasma cell that secretes antibodies specific to the antigen that was originally bound. Plasma cells do not last long in the circulation, this is in contrast to memory cells that last for very long periods of time.

The term “peripheral blood” refers to blood which travels through peripheral vessels. Peripheral blood is typically obtained by venipuncture (also called phlebotomy), or by finger prick for small quantities.

The term “plasma hyperimmune” refers to a polyclonal antibody preparation similar to intravenous immunoglobulin (IVIg), except that it is prepared from the plasma of donors with high titers of antibody against a specific organism or antigen. The term hyperimmune is often used interchangeably with the terms “hyperimmune gammaglobulin” and “hyperimmune globulin”. Some agents against which hyperimmune globulins are available include hepatitis B, rabies, tetanus toxin, varicella-zoster, etc. Administration of hyperimmune globulin provides “passive” immunity to the patient against an agent. This is in contrast to vaccines that provide “active” immunity. However, vaccines take much longer to achieve that purpose while hyperimmune globulin provides instant “passive” short-lived immunity.

The term “activity” refers to a quantitative measurement of an ABP or antibody against an antigen, vaccine, protein, epitope, cell, bacterium, or virus. Activity can be assessed using in vivo or in vitro methods.

The term “recombinant” refers to proteins that result from the expression of recombinant DNA within living cells. Recombinant DNA is the general name for a piece of DNA that has been created by the combination of at least two separate segments of DNA.

The term “neutralization” refers to the ability of specific antibodies to block the site(s) on viruses that they use to enter their target cell. The effect of a neutralizing antibody can be negligible even with large excesses of antibody production if they lack specificity to this antigen. The production of specific antibodies can be learned for a faster response at next exposition. The reduction or destruction of a homologous infectious agent can be partial or complete and can make it no longer infectious or pathogenic to other cells.

A “variant” of a polypeptide (e.g., an antibody) comprises an amino acid sequence wherein one or more amino acid residues are inserted into, deleted from and/or substituted into the amino acid sequence relative to the native polypeptide sequence, and retains essentially the same biological activity as the native polypeptide. The biological activity of the polypeptide can be measured using standard techniques in the art (for example, if the variant is an antibody, its activity may be tested by binding assays, as described herein). Variants of the invention include fragments, analogs, recombinant polypeptides, synthetic polypeptides, and/or fusion proteins.

A “derivative” of a polypeptide is a polypeptide (e.g., an antibody) that has been chemically modified, e.g., via conjugation to another chemical moiety such as, for example, polyethylene glycol, albumin (e.g., human serum albumin), phosphorylation, and glycosylation. Unless otherwise indicated, the term “antibody” includes, in addition to antibodies comprising two full-length heavy chains and two full-length light chains, derivatives, variants, fragments, and muteins thereof, examples of which are described below.

A nucleotide sequence is “operably linked” to a regulatory sequence if the regulatory sequence affects the expression (e.g., the level, timing, or location of expression) of the nucleotide sequence. A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, CA and Baron et al., 1995, Nucleic Acids Res. 23:3605-06.

A “host cell” is a cell that can be used to express a nucleic acid, e.g., a nucleic acid of the invention. A host cell can be a prokaryote, for example, E. coli, or it can be a eukaryote, for example, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Examples of host cells include CS-9 cells, the COS-7 line of monkey kidney cells (ATCC CRL 1651) (see Gluzman et al., 1981, Cell 23:175), L cells, C127 cells, 3T3 cells (ATCC CCL 163), Chinese hamster ovary (CHO) cells or their derivatives such as Veggie CHO and related cell lines which grow in serum-free media (see Rasmussen et al., 1998, Cytotechnology 28:31), HeLa cells, BHK (ATCC CRL 10) cell lines, the CV1/EBNA cell line derived from the African green monkey kidney cell line CV1 (ATCC CCL 70) (see McMahan et al., 1991, EMBO J. 10:2821), human embryonic kidney cells such as 293, 293 EBNA or MSR 293, human epidermal A431 cells, human Colo205 cells, other transformed primate cell lines, normal diploid cells, cell strains derived from in vitro culture of primary tissue, primary explants, HL-60, U937, HaK or Jurkat cells. Typically, a host cell is a cultured cell that can be transformed or transfected with a polypeptide-encoding nucleic acid, which can then be expressed in the host cell.

The phrase “recombinant host cell” can be used to denote a host cell that has been transformed or transfected with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but to the progeny or potential progeny of such a cell. Because certain modifications may occur in succeeding generations due to, e.g., mutation or environmental influence, such progeny may not, in fact, be identical to the parent cell, but are still included within the scope of the term as used herein.

### 7.2. Other Interpretational Conventions

Ranges recited herein are understood to be shorthand for all of the values within the range, inclusive of the recited endpoints. For example, a range of 1 to 50 is understood to include any number, combination of numbers, or sub-range from the group consisting of 1, 2, 3 4 5 6 7, 8 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless otherwise indicated, reference to a compound that has one or more stereocenters intends each stereoisomer, and all combinations of stereoisomers, thereof.

### 7.3. Pharmaceutical Composition of Recombinant Antibodies

The present disclosure provides a pharmaceutical composition comprising: at least 100 unique human recombinant antibodies specifically binding to a Hepatitis B virus (HBV) surface antigen; and a pharmaceutically acceptable excipient, wherein each of the recombinant antibodies comprises a cognate pair of heavy chain and light chain variable regions from a single cell out of a blood sample from a donor exposed to the HBV surface antigen; and has been generated from a polynucleotide construct encoding the cognate pair of heavy chain and light chain variable regions.

The recombinant antibodies specifically bind one or more HBV antigens.

In some embodiments, the at least 100 recombinant antibodies have EC50 against the HBV surface antigen lower than 1 μg/ml. In some embodiments, the at least 100 recombinant antibodies have EC50 against the HBV surface antigen lower than 0.5 μg/ml, 0.1 μg/ml, 0.05 μg/ml, 0.01 μg/ml, 0.005 μg/ml or 0.001 μg/ml.

In some embodiments, the at least 100 recombinant antibodies have at least 100-fold lower EC50 against the HBV surface antigen compared to a plasma sample from the donor exposed to the HBV surface antigen. In some embodiments, the at least 100 recombinant antibodies have at least 500-fold, 1,000-fold, 1,500-fold, 2,000-fold, 3,000-fold, 4,000-fold, or 5,000-fold lower EC50 against the HBV surface antigen compared to a plasma sample from the donor exposed to the HBV surface antigen.

In some embodiments, the at least 100 recombinant antibodies have at least 100-fold lower EC50 against the HBV surface antigen compared to HyperHEP B®. In some embodiments, the at least 100 recombinant antibodies have at least 500-fold, 1,000-fold, 1,500-fold, 2,000-fold, 3,000-fold, 4,000-fold, or 5,000-fold lower EC50 against the HBV surface antigen compared to HyperHEP B®.

In some embodiments, the EC50 was measured by ELISA.

In some embodiments, the at least 100 recombinant antibodies have IC50 against HBV lower than 0.5 μg/ml. In some embodiments, the at least 100 recombinant antibodies have IC50 against HBV lower than 0.1 μg/ml, 0.05 μg/ml, 0.01 μg/ml, 0.005 μg/ml, or 0.001 μg/ml. In some embodiments, the at least 100 recombinant antibodies have at least 100-fold lower IC50 against HBV compared to a plasma sample from the donor exposed to the HBV surface antigen. In some embodiments, the at least 100 recombinant antibodies have at least 500-fold 1,000-fold, 2,000-fold, 3,000-fold, 4,000-fold, 5,000-fold, 10,000-fold, 20,000-fold, 30,000-fold, 40,000-fold or 50,000-fold lower IC50 against HBV compared to a plasma sample from the donor exposed to the HBV surface antigen.

In some embodiments, the at least 100 recombinant antibodies have at least 100-fold lower IC50 against HBV compared to HyperHEP B®. In some embodiments, the at least 100 recombinant antibodies have at least 500-fold 1,000-fold, 2,000-fold, 3,000-fold, 4,000-fold, 5,000-fold, 10,000-fold, 20,000-fold, 30,000-fold, 40,000-fold or 50,000-fold lower IC50 against HBV compared to HyperHEP B®.

In some embodiments, the IC50 against HBV was measured by detecting neutralization of live HBV infection to human hepatocyte cells. In some embodiments, the IC50 is measured in vitro or in vivo.

In some embodiments, the at least 100 recombinant antibodies can neutralize an HBV variant or serotype that a plasma sample from the donor exposed to the HBV surface antigen cannot neutralize. In some embodiments, the at least 100 recombinant antibodies can neutralize more HBV variants or serotypes than a plasma sample from the donor exposed to the HBV surface antigen cannot neutralize.

In some embodiments, the at least 100 recombinant antibodies can neutralize an HBV variant or serotype that a monoclonal antibody to an HBV surface antigen cannot neutralize. In some embodiments, the at least 100 recombinant antibodies can neutralize more HBV variants or serotypes than a monoclonal antibody to an HBV surface antigen.

In some embodiments, the pharmaceutical composition comprises a plurality of recombinant antibodies specifically binding to one or more HBV antigens.

In some embodiments, the pharmaceutical composition comprises at least 500, 1,000, 2,000, 3,000, 4,000, 5,000, 10,000, 100,000, or more unique human recombinant antibodies specifically binding to an HBV surface antigen.

In some embodiments, the donor has been exposed to HBV. In some embodiments, the donor has been vaccinated against the HBV surface antigen.

In some embodiments, the at least 100 unique human recombinant antibodies comprise cognate pairs of heavy chain and light chain variable regions from single cells from at least two donors. In some embodiments, the at least 100 unique human recombinant antibodies comprise cognate pairs of heavy chain and light chain variable regions from single cells from at least three, four, five, six, seven, eight, nine or ten donors. In some embodiments, the at least 100 unique human recombinant antibodies comprise cognate pairs of heavy chain and light chain variable regions from single cells from one to ten donors, from two to eight donors, from four to six donors, or more than ten donors. In certain embodiments, rHBIG is a recombinant polyclonal antibody comprising antibodies derived from one, two, four, eight, twelve, 50, or 100 human donors. In certain embodiments, rCIG is a recombinant polyclonal antibody comprising, 10, 100, 1,000, 10,000, 100,000, or more than 100,000 distinct antibody sequences.

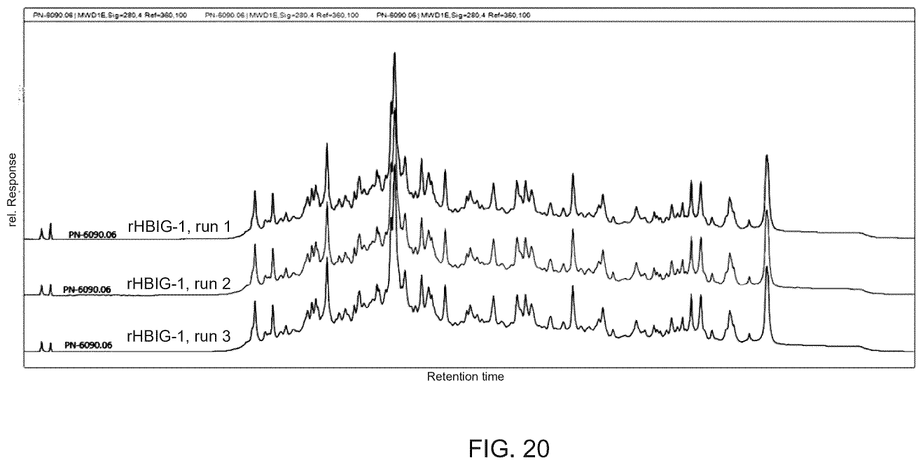

In some embodiments, rHBIG is a recombinant polyclonal antibody comprising a CEX-HPLC peak distribution having at least 80%, 90%, 95%, 98%, or 99% overlap with the distribution in FIG. 20 in terms of peak sizes, peak retention times, or peak numbers, wherein the CEX-HPLC is performed with a pH gradient from a low pH (pH 5-6) mobile phase A to a high pH (pH 10-11) mobile phase B. In some embodiments, rHBIG is a recombinant polyclonal antibody comprising a CEX-HPLC peak distribution having at least 80%, 90%, 95%, 98%, or 99% overlap with the distribution in FIG. 20 in terms of peak sizes, peak retention times, or peak numbers, wherein the CEX-HPLC is performed with a pH gradient from a low pH (pH 5.6) mobile phase A to a high pH (pH 10.2) mobile phase B.

In some embodiments, rHBIG is a recombinant polyclonal antibody comprising a CEX-HPLC peak distribution having at least 80%, 90%, 95%, 98%, or 99% overlap with the distribution in FIG. 20 in terms of peak sizes, peak retention times, and peak numbers, wherein the CEX-HPLC is performed with a pH gradient from a low pH (pH 5-6) mobile phase A to a high pH (pH 10-11) mobile phase B. In some embodiments, rHBIG is a recombinant polyclonal antibody comprising a CEX-HPLC peak distribution having at least 80%, 90%, 95%, 98%, or 99% overlap with the distribution in FIG. 20 in terms of peak sizes, peak retention times, and peak numbers, wherein the CEX-HPLC is performed with a pH gradient from a low pH (pH 5.6) mobile phase A to a high pH (pH 10.2) mobile phase B.

In some embodiments, the CEX-HPLC is performed under the condition provided in the below table.

HPLC Condition

| | |
|---|---|
| Mobile Phase A | CX-1 pH Gradient Buffer A, 1X (pH 5.6) |
| Mobile Phase B | CX-1 pH Gradient Buffer B, 1X (pH 10.2) |
| Column | Proteomix SCX NP1.7 4.6 × 100 mm |
| Flow Rate | 0.80 mL/min or specified |
| Column Temperature | 30° C. |
| Gradient Range | 10-100% B or specified |
| Gradient Time | 35 min or specified |

In some embodiments, rHBIG is a recombinant polyclonal antibody comprising a CEX-HPLC peak distribution having a retention time ranging from 10-55 minutes. In certain embodiments, rHBIG is a recombinant polyclonal antibody comprising a CEX-HPLC peak distribution having a retention time ranging from 20-30 minutes. In certain embodiments, rHBIG is a recombinant polyclonal antibody comprising a CEX-HPLC peak distribution having a retention time ranging from 22-28 minutes. In some embodiments, rHBIG is a recombinant polyclonal antibody comprising a CEX-HPLC peak distribution having a retention time of at least 10 minutes, at least 15 minutes, at least 20 minutes, at least 25 minutes, at least 30 minutes, at least 35 minutes, at least 40 minutes, at least 45 minutes, at least 50 minutes, or at least 55 minutes.

In certain embodiments, rHBIG is a recombinant polyclonal antibody comprising antibodies at various molar ratios, or antibodies comprising substantially similar molar ratios. In certain embodiments, these recombinant polyclonal antibodies comprise therapeutics for Hepatitis B. In other embodiments, any single antibody from the recombinant polyclonal antibody is used as a therapeutic for Hepatitis B. In certain embodiments, any single antibody or recombinant polyclonal antibody is used as a therapeutic for any kind of HBV infection in human patients.

In some embodiments, the donor has been selected based on their immune response to the antigen. In some embodiments the donor has been selected based on its antibody profile against HBV antigen. In some embodiments the donor has been selected based on its antibody titer.

In some embodiments, each of the recombinant antibodies is an IgG antibody. In some embodiments, each of the recombinant antibodies is an IgM antibody. In some embodiments, each of the recombinant antibodies is an IgG or IgM antibody. In some embodiments, each of the recombinant antibodies is an IgA antibody. In some embodiments, each of the recombinant antibodies is a human IgG1 subtype. In some embodiments, each of the recombinant antibodies is a human IgG2 subtype. In some embodiments, each of the recombinant antibodies is a human IgG3 subtype. In some embodiments, each of the recombinant antibodies is a human IgG4 subtype.

In some embodiments, the recombinant antibodies comprise scFvs. In some embodiments, the recombinant antibodies consist of scFvs. In some embodiments, the recombinant antibodies comprise antibody fragments. In some embodiments, the recombinant antibodies consist of antibody fragments. In some embodiments, the recombinant antibodies are recombinant full-length antibodies. In some embodiments, the recombinant antibodies comprise recombinant full-length antibodies. In some embodiments, the recombinant antibodies comprise human antibodies. In some embodiments, the recombinant antibodies comprise humanized antibodies. In some embodiments, the recombinant antibodies comprise monospecific antibodies. In some embodiments, the recombinant antibodies comprise bispecific antibodies. In some embodiments, the recombinant antibodies are a human IgG1 subtype. In some embodiments, the recombinant antibodies comprises IgGs, IgAs, or IgMs.

In some embodiments, the recombinant antibodies comprise antibody fragments. The the recombinant antibodies can be a Fab fragment, a F(ab′)₂ fragment an Fv fragment, or a combination thereof. A Fab fragment is a monovalent fragment having the V_{L}, V_{H}, C_{L} and C_{H1} domains; a F(ab′)₂ fragment is a bivalent fragment having two Fab fragments linked by a disulfide bridge at the hinge region; a Fd fragment has the V_{H} and C_{H1} domains; an Fv fragment has the V_{L} and V_{H} domains of a single arm of an antibody; and a dAb fragment has a V_{H} domain, a V_{L} domain, or an antigen-binding fragment of a V_{H} or V_{L} domain (U.S. Pat. Nos. 6,846,634, 6,696,245, US App. Pub. No. 05/0202512, 04/0202995, 04/0038291, 04/0009507, 03/0039958, Ward et al., Nature 341:544-546, 1989).

Naturally occurring immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4. The assignment of amino acids to each domain is in accordance with the definitions of Kabat et al. in Sequences of Proteins of Immunological Interest, 5th Ed., US Dept. of Health and Human Services, PHS, NIH, NIH Publication no. 91-3242, 1991.

In some embodiments, the recombinant antibodies comprise humanized antibodies. A humanized antibody has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

Fragments or analogs of antibodies can be readily prepared by those of ordinary skill in the art following the teachings of this specification and using techniques well-known in the art. Preferred amino- and carboxy-termini of fragments or analogs occur near boundaries of functional domains. Structural and functional domains can be identified by comparison of the nucleotide and/or amino acid sequence data to public or proprietary sequence databases. Computerized comparison methods can be used to identify sequence motifs or predicted protein conformation domains that occur in other proteins of known structure and/or function. Methods to identify protein sequences that fold into a known three-dimensional structure are known. See, e.g., Bowie et al., 1991, Science 253:164.

The recombinant antibodies can also be any synthetic or genetically engineered protein. For example, antibody fragments include isolated fragments consisting of the light chain variable region, “Fv” fragments consisting of the variable regions of the heavy and light chains, recombinant single chain polypeptide molecules in which light and heavy variable regions are connected by a peptide linker (scFv proteins).

Another form of an antibody fragment is a peptide comprising one or more complementarity determining regions (CDRs) of an antibody. CDRs (also termed “minimal recognition units”, or “hypervariable region”) can be incorporated into a molecule either covalently or noncovalently to make it an antigen binding protein. CDRs can be obtained by constructing polynucleotides that encode the CDR of interest. Such polynucleotides are prepared, for example, by using the polymerase chain reaction to synthesize the variable region using mRNA of antibody producing cells as a template (see, for example, Larrick et al., Methods: A Companion to Methods in Enzymology 2:106, 1991; Courtenay Luck, “Genetic Manipulation of Monoclonal Antibodies,” in Monoclonal Antibodies: Production, Engineering and Clinical Application, Ritter et al. (eds.), page 166 (Cambridge University Press 1995); and Ward et al., “Genetic Manipulation and Expression of Antibodies,” in Monoclonal Antibodies: Principles and Applications, Birch et al., (eds.), page 137 (Wiley Liss, Inc. 1995).

The variable region domains of recombinant polyclonal antibodies can be any naturally occurring variable domain or an engineered version thereof. By engineered version is meant a variable region domain that has been created using recombinant DNA engineering techniques. Such engineered versions include those created, for example, from a specific antibody variable region by insertions, deletions, or changes in or to the amino acid sequences of the specific antibody. Particular examples include engineered variable region domains containing at least one CDR and optionally one or more framework amino acids from a first antibody and the remainder of the variable region domain from a second antibody.

The variable region domain may be covalently attached at a C terminal amino acid to at least one other antibody domain or a fragment thereof. Thus, for example, a V_{H} domain that is present in the variable region domain may be linked to an immunoglobulin CH1 domain, or a fragment thereof. Similarly, a V_{L} domain may be linked to a CK domain or a fragment thereof. In this way, for example, the antibody may be a Fab fragment wherein the antigen binding domain contains associated V_{H} and V_{L} domains covalently linked at their C termini to a CH1 and CK domain, respectively. The CH1 domain may be extended with further amino acids, for example to provide a hinge region or a portion of a hinge region domain as found in a Fab′ fragment, or to provide further domains, such as antibody CH2 and CH3 domains.

The recombinant polyclonal antibody provided herein can include the cognate pairs of heavy and light chain CDR3 sequence disclosed herein. For example, CDRs may be incorporated into known antibody framework regions (IgG1, IgG2, etc.), or conjugated to a suitable vehicle to enhance the half-life thereof. Suitable vehicles include, but are not limited to Fc, polyethylene glycol (PEG), albumin, transferrin, and the like. These and other suitable vehicles are known in the art. Such conjugated CDR peptides may be in monomeric, dimeric, tetrameric, or other form. In one embodiment, one or more water-soluble polymer is bonded at one or more specific position, for example at the amino terminus, of a binding agent.

In certain embodiments, the recombinant polyclonal antibody comprises one or more water soluble polymer attachments, including, but not limited to, polyethylene glycol, polyoxyethylene glycol, or polypropylene glycol. See, e.g., U.S. Pat. Nos. 4,640,835, 4,496,689, 4,301,144, 4,670,417, 4,791,192 and 4,179,337. In certain embodiments, a derivative binding agent comprises one or more of monomethoxy-polyethylene glycol, dextran, cellulose, or other carbohydrate based polymers, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of such polymers. In certain embodiments, one or more water-soluble polymer is randomly attached to one or more side chains. In certain embodiments, PEG can act to improve the therapeutic capacity for a binding agent, such as an antibody. Certain such methods are discussed, for example, in U.S. Pat. No. 6,133,426, which is hereby incorporated by reference for any purpose.

A recombinant polyclonal antibody can have, for example, the structure of a naturally occurring immunoglobulin. An “immunoglobulin” is a tetrameric molecule. In a naturally occurring immunoglobulin, each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site such that an intact immunoglobulin has two binding sites.

Different antibodies may bind to different domains of disease targets or act by different mechanisms of action. As indicated herein inter alia, the domain regions are designated such as to be inclusive of the group, unless otherwise indicated. For example, amino acids 4-12 refers to nine amino acids: amino acids at positions 4, and 12, as well as the seven intervening amino acids in the sequence. Other examples include antigen binding proteins that inhibit binding of a pathogen to its target cell, i.e., neutralizing activity. An antigen binding protein need not completely inhibit a binding to target cell to find use in the present invention.

The antibodies provided herein can induce various biological effects associated with binding to an antigen that comprises a vaccine. In some embodiments, an antibody provided herein prevents binding of a virus to a cell, which therein prevents entry of the virus into the cell. In some embodiments, the antibody binds to the cell surface of a patient's cells, in order to eliminate cells associated with a pathology.

The antibodies describe herein can include an Fc region, e.g., a dimer Fc polypeptide. One suitable Fc polypeptide, described in PCT application WO 93/10151 (hereby incorporated by reference), is a single chain polypeptide extending from the N-terminal hinge region to the native C-terminus of the Fc region of a human IgG1 antibody. Another useful Fc polypeptide is the Fc mutein described in U.S. Pat. No. 5,457,035 and in Baum et al., 1994, EMBO J. 13:3992-4001. The amino acid sequence of this mutein is identical to that of the native Fc sequence presented in WO 93/10151, except that amino acid 19 has been changed from Leu to Ala, amino acid 20 has been changed from Leu to Glu, and amino acid 22 has been changed from Gly to Ala. The mutein exhibits reduced affinity for Fc receptors.

Antigen-binding fragments of antibodies of the invention can be produced by conventional techniques. Examples of such fragments include, but are not limited to, Fab and F(ab′)₂ fragments. Antibody fragments and derivatives produced by genetic engineering techniques are also contemplated.

Additional embodiments include chimeric antibodies, e.g., humanized versions of non-human (e.g., murine) monoclonal antibodies. Such humanized antibodies may be prepared by known techniques and offer the advantage of reduced immunogenicity when the antibodies are administered to humans. In one embodiment, a humanized antibody comprises the variable domain of a murine antibody (or all or part of the antigen binding site thereof) and a constant domain derived from a human antibody. Alternatively, a humanized antibody fragment may comprise the antigen binding site of a murine antibody and a variable domain fragment (lacking the antigen-binding site) derived from a human antibody. Procedures for production of chimeric and further engineered antibodies include those described in Riechmann et al., 1988, Nature 332:323, Liu et al., 1987, Proc. Nat. Acad. Sci. USA 84:3439, Larrick et al., 1989, Bio/Technology 7:934, and Winter et al., 1993, TIPS 14:139. In one embodiment, the chimeric antibody is a CDR grafted antibody. Techniques for humanizing antibodies are discussed in, e.g., U.S. Pat. Nos. 5,869,619, 5,225,539, 5,821,337, 5,859,205, 6,881,557, Padlan et al., 1995, FASEB J. 9:133-39, and Tamura et al., 2000, J. Immunol. 164:1432-41.

Procedures have been developed for generating human or partially human antibodies in non-human animals. For example, mice in which one or more endogenous immunoglobulin genes have been inactivated by various means have been prepared. Human immunoglobulin genes have been introduced into the mice to replace the inactivated mouse genes. Antibodies produced in the animal incorporate human immunoglobulin polypeptide chains encoded by the human genetic material introduced into the animal. In one embodiment, a non-human animal, such as a transgenic mouse, is immunized with a vaccine, such that antibodies directed against the vaccine antigen are generated in the animal.

Examples of techniques for production and use of transgenic animals for production of human or partially human antibodies are described in U.S. Pat. Nos. 5,814,318, 5,569,825, and 5,545,806, Davis et al., 2003, Production of human antibodies from transgenic mice in Lo, ed. Antibody Engineering: Methods and Protocols, Humana Press, NJ:191-200, Kellermann et al., 2002, Curr Opin Biotechnol. 13:593-97, Russel et al., 2000, Infect Immun. 68:1820-26, Gallo et al., 2000, Eur J Immun. 30:534-40, Davis et al., 1999, Cancer Metastasis Rev. 18:421-25, Green, 1999, J Immunol Methods. 231:11-23, Jakobovits, 1998, Advanced Drug Delivery Reviews 31:33-42, Green et al., 1998, J Exp Med. 188:483-95, Jakobovits A, 1998, Exp. Opin. Invest. Drugs. 7:607-14, Tsuda et al., 1997, Genomics. 42:413-21, Mendez et al., 1997, Nat Genet. 15:146-56, Jakobovits, 1994, Curr Biol. 4:761-63, Arbones et al., 1994, Immunity. 1:247-60, Green et al., 1994, Nat Genet. 7:13-21, Jakobovits et al., 1993, Nature. 362:255-58, Jakobovits et al., 1993, Proc Natl Acad Sci USA. 90:2551-55. Chen, J., M. Trounstine, F. W. Alt, F. Young, C. Kurahara, J. Loring, D. Huszar. Inter'l Immunol. 5 (1993): 647-656, Choi et al., 1993, Nature Genetics 4: 117-23, Fishwild et al., 1996, Nature Biotech. 14: 845-51, Harding et al., 1995, Annals of the New York Academy of Sciences, Lonberg et al., 1994, Nature 368: 856-59, Lonberg, 1994, Transgenic Approaches to Human Monoclonal Antibodies in Handbook of Experimental Pharmacology 113: 49-101, Lonberg et al., 1995, Internal Review of Immunology 13: 65-93, Neuberger, 1996, Nature Biotechnology 14: 826, Taylor et al., 1992, Nucleic Acids Res. 20: 6287-95, Taylor et al., 1994, Inter'l Immunol. 6: 579-91, Tomizuka et al., 1997, Nature Genetics 16: 133-43, Tomizuka et al., 2000, Pro. Nat'l Acad. Sci. USA 97: 722-27, Tuaillon et al., 1993, Pro. Nat'l Acad. Sci. USA 90: 3720-24, and Tuaillon et al., 1994, J. Immunol. 152: 2912-20.

Antibodies of the invention can comprise any constant region known in the art. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. In one embodiment, the light or heavy chain constant region is a fragment, derivative, variant, or mutein of a naturally occurring constant region.

Techniques are known for deriving an antibody of a different subclass or isotype from an antibody of interest, i.e., subclass switching. Thus, IgG antibodies may be derived from an IgM antibody, for example, and vice versa. Such techniques allow the preparation of new antibodies that possess the antigen-binding properties of a given antibody (the parent antibody), but also exhibit biological properties associated with an antibody isotype or subclass different from that of the parent antibody. Recombinant DNA techniques may be employed. Cloned DNA encoding particular antibody polypeptides may be employed in such procedures, e.g., DNA encoding the constant domain of an antibody of the desired isotype. See also Lantto et al., 2002, Methods Mol. Biol. 178:303-16.

Single chain antibodies may be formed by linking heavy and light chain variable domain (Fv region) fragments via an amino acid bridge (short peptide linker, e.g., a synthetic sequence of amino acid residues), resulting in a single polypeptide chain. Such single-chain Fvs (scFvs) have been prepared by fusing DNA encoding a peptide linker between DNAs encoding the two variable domain polypeptides (V_{L} and V_{H}). The resulting polypeptides can fold back on themselves to form antigen-binding monomers, or they can form multimers (e.g., dimers, trimers, or tetramers), depending on the length of a flexible linker between the two variable domains (Kortt et al., 1997, Prot. Eng. 10:423; Kortt et al., 2001, Biomol. Eng. 18:95-108, Bird et al., 1988, Science 242:423-26 and Huston et al., 1988, Proc. Natl. Acad. Sci. USA 85:5879-83). By combining different V_{L} and V_{H}-comprising polypeptides, one can form multimeric scFvs that bind to different epitopes (Kriangkum et al., 2001, Biomol. Eng. 18:31-40). Techniques developed for the production of single chain antibodies include those described in U.S. Pat. No. 4,946,778; Bird, 1988, Science 242:423; Huston et al., 1988, Proc. Natl. Acad. Sci. USA 85:5879; Ward et al., 1989, Nature 334:544, de Graaf et al., 2002, Methods Mol Biol. 178:379-87.

An antibody or polyclonal antibodies according to the invention may have a binding affinity for antigen target of less than or equal to 5×10⁻⁷M, less than or equal to 1×10⁻⁷M, less than or equal to 0.5×10⁻⁷M, less than or equal to 1×10⁻⁸M, less than or equal to 1×10⁻⁹M, less than or equal to 1×10⁻¹⁰M, less than or equal to 1×10⁻¹¹M, or less than or equal to 1×10⁻¹²M.

The affinity of an antibody or recombinant polyclonal antibodies can be determined by one of ordinary skill in the art using conventional techniques, for example those described by Scatchard et al. (Ann. N. Y. Acad. Sci. 51:660-672 (1949)) or by surface plasmon resonance (SPR; BIAcore, Biosensor, Piscataway, NJ). For surface plasmon resonance, target molecules are immobilized on a solid phase and exposed to ligands in a mobile phase running along a flow cell. If ligand binding to the immobilized target occurs, the local refractive index changes, leading to a change in SPR angle, which can be monitored in real time by detecting changes in the intensity of the reflected light. The rates of change of the SPR signal can be analyzed to yield apparent rate constants for the association and dissociation phases of the binding reaction. The ratio of these values gives the apparent equilibrium constant (affinity) (see, e.g., Wolff et al., Cancer Res. 53:2560-65 (1993)).

In some embodiments, the pharmaceutical composition comprises a therapeutically or prophylactically effective amount of the polypeptides or proteins in a mixture with pharmaceutically acceptable materials. In some embodiments, the pharmaceutical composition comprises recombinant polyclonal antibodies produced using sequences derived from immune cells of a donor exposed to one or more HBV antigens. In some embodiments, the pharmaceutical composition comprises antibodies produced using sequences derived from immune cells of multiple donors exposed to one or more HBV antigens. The donors can be a human, non-human animal (mouse, humanized mouse, rat, humanized rat, horse, or cow) or both.

In some embodiments, the pharmaceutical composition comprises first polyclonal antibodies comprising a first antibody, wherein each of the first polyclonal antibodies specifically binds to a first antigen, and second polyclonal antibodies comprising a second antibody, wherein each of the second polyclonal antibodies specifically binds to a second antigen. In some embodiments, the first antigen is an antigen of HBV. In some embodiments, the second antigen is a different antigen of HBV.

In some embodiments, the pharmaceutical composition comprises about 10, 100, 500, 1,000, 5,000, 10,000, 50,000 or more than 100,000 distinct antibodies, each having a unique sequence. In some embodiments, the pharmaceutical composition comprises at least 10, 100, 500, 1,000, 5,000, 10,000, 20,000, 30,000, 40,000, 50,000 or more than 100,000 distinct antibodies, each having a unique sequence.

In some embodiments, the recombinant polyclonal antibodies described herein clear and neutralize the virus and viral particles in patients with hepatitis B virus (HBV) infection by ADCC and/or ADCP, for example mediated by binding of NK cell-expressed CD16 to the polyclonal antibody Fc domain. In some embodiments, the recombinant polyclonal antibodies described herein is bound by surface expressed Fc receptors, when complexed with one or more antigens, which induces ADCC and ADCP linked downstream signaling. In some embodiments, the recombinant polyclonal antibody in complex with HBsAg is bound by human immune cell cohorts expressing FcγRs.

The pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition.

In some embodiments, the pharmaceutical composition comprises a compound selected from benzalkonium chloride, benzyl alcohol, benzoic acid, salicylic acid, thimerosal, phenethyl alcohol, methylparaben, propylparaben, chlorhexidine, sorbic acid, and hydrogen peroxide in an amount effective to preserve the pharmaceutical composition. In some embodiments, the composition is free of preservatives. In some embodiments, the composition is free of alive or dead HBV or any protein or polynucleotide produced from HBV.

Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates, other organic acids); bulking agents (such as mannitol or glycine), chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (preferably sodium or potassium chloride, mannitol sorbitol); delivery vehicles; diluents; excipients and/or pharmaceutical adjuvants. Neutral buffered saline or saline mixed with conspecific serum albumin are examples of appropriate diluents. In accordance with appropriate industry standards, preservatives such as benzyl alcohol may also be added. The composition may be formulated as a lyophilizate using appropriate excipient solutions (e.g., sucrose) as diluents. Suitable components are nontoxic to recipients at the dosages and concentrations employed. Further examples of components that may be employed in pharmaceutical formulations are presented in Remington's Pharmaceutical Sciences, 16^{th} Ed. (1980) and 20^{th} Ed. (2000), Mack Publishing Company, Easton, PA.

Optionally, the composition additionally comprises one or more physiologically active agents, for example, an anti-viral agent, plasma IVIg, etc. In various embodiments, the composition comprises one, two, three, four, five, or six physiologically active agents in addition to an antibody.

In another embodiment of the invention, the compositions disclosed herein may be formulated in a neutral or salt form. Illustrative pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective.

The carriers can further comprise any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically-acceptable” refers to molecular entities and compositions that do not produce an allergic or similar untoward reaction when administered to a human.

The optimal pharmaceutical composition will be determined by one skilled in the art depending upon, for example, the intended route of administration, delivery format, and desired dosage. See for example, Remington's Pharmaceutical Sciences, supra. Such compositions may influence the physical state, stability, rate of in vivo release, and rate of in vivo clearance of the polypeptide. For example, suitable compositions may be water for injection, physiological saline solution for parenteral administration.

### Content of Pharmaceutically Active Ingredient

In typical embodiments, the active ingredient (i.e., the proteins and polypeptides of the present invention) is present in the pharmaceutical composition at a concentration of at least 0.01 mg/ml, at least 0.1 mg/ml, at least 0.5 mg/ml, or at least 1 mg/ml. In certain embodiments, the active ingredient is present in the pharmaceutical composition at a concentration of at least 1 mg/ml, 2 mg/ml, 3 mg/ml, 4 mg/ml, 5 mg/ml, 10 mg/ml, 15 mg/ml, 20 mg/ml, or 25 mg/ml. In certain embodiments, the active ingredient is present in the pharmaceutical composition at a concentration of at least 30 mg/ml, 35 mg/ml, 40 mg/ml, 45 mg/ml or 50 mg/ml. In certain embodiments, the active ingredient is present in the pharmaceutical composition at a concentration of at least 100 mg/ml, 250 mg/ml, 500 mg/ml, 750 mg/ml, 1 g/ml, 5 g/ml, 10 g/ml, or 50 g/ml. In certain embodiments, the active ingredient is present in the pharmaceutical composition at a concentration of 1 mg/mL to 50 mg/mL, 1 mg/mL to 25 mg/mL, 1 mg/mL to 15 mg/mL or 1 mg/mL to 10 mg/mL.

### Formulation Generally

The pharmaceutical composition can be in any form appropriate for human or veterinary medicine, including a liquid, an oil, an emulsion, a gel, a colloid, an aerosol or a solid.

The pharmaceutical composition can be formulated for administration by any route of administration appropriate for human or veterinary medicine, including enteral and parenteral routes of administration.

In some embodiments, the pharmaceutical composition is formulated for intravenous, intraperitoneal, intramuscular, or subcutaneous administration. In some embodiments, the pharmaceutical composition is formulated for intravenous injection or intravenous infusion.

In some embodiments, the pharmaceutical composition is formulated for intrathecal or intracerebroventricular administration.

In some embodiments, the pharmaceutical composition is formulated for topical administration.

### Pharmacological Compositions Adapted for Injection

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required. In some embodiments, the isotonic vehicle is sodium chloride. In some embodiments, the pharmaceutical composition comprises a unit dose of the pharmaceutical composition. In some embodiments, the unit dose comprises at least 0.1% sodium Chloride, at least 0.2% sodium Chloride, at least 0.3% sodium Chloride, at least 0.4% sodium Chloride, at least 0.5% sodium Chloride, at least 0.6% sodium Chloride, at least 0.7% sodium Chloride, at least 0.8% sodium Chloride, at least 0.9% sodium Chloride, and at least 1% sodium Chloride.

In some embodiments the formulation has a pH 4-7. In some embodiments the formulation has a pH4. In some embodiments the formulation has a pH4.5. In some embodiments the formulation has a pH5. In some embodiments the formulation has a pH5.5. In some embodiments the formulation has a pH6. In some embodiments the formulation has a pH6.5. In some embodiments the formulation has a pH7.

In some embodiments the formulation comprises 20 mM succinate. In some embodiments the formulation comprises 20 mM acetate. In some embodiments the formulation comprises 20 mM histidine. In some embodiments the formulation comprises 30 mM succinate. In some embodiments the formulation comprises 30 mM acetate. In some embodiments the formulation comprises 30 mM histidine. In some embodiments the formulation comprises 10 mM succinate. In some embodiments the formulation comprises 10 mM acetate. In some embodiments the formulation comprises 10 mM histidine.

In some embodiments, the formulation comprises PS80, PS20 or P188. In some embodiments, the formulation comprises PS80, PS20 and P188. In some embodiments, the formulation comprises 0.02% PS80, 0.02% PS20, or 0.1% P188.

In some embodiments, the unit dose comprises the ABP at a concentration of 1 mg/mL to 100 mg/ml. In some embodiments, the unit dose comprises the ABP at a concentration of 1 mg/mL to 50 mg/mL, 1 mg/mL to 25 mg/mL, 1 mg/mL to 15 mg/mL or 1 mg/mL to 10 mg/mL

In various embodiments, the unit dosage form is a vial, ampule, bottle, or pre-filled syringe. In some embodiments, the unit dosage form contains 0.01 mg, 0.1 mg, 0.5 mg, 1 mg, 2.5 mg, 5 mg, 10 mg, 12.5 mg, 25 mg, 50 mg, 75 mg, or 100 mg of the active ingredient. In some embodiments, the unit dosage form contains 125 mg, 150 mg, 175 mg, or 200 mg of the active ingredient. In some embodiments, the unit dosage form contains at least 250 mg, 1 g, 10 g, 20 g, 30 g, 40 g, 50 g, 60 g, 70 g, 80 g, 90 g, or 100 g of the active ingredient. In some embodiments, the unit dosage form contains about 250 mg, 1 g, 10 g, 20 g, 30 g, 40 g, 50 g, 60 g, 70 g, 80 g, 90 g, or 100 g of the active ingredient. In some embodiments, the unit dosage form contains 50 mg, 100 mg, 150 mg, 200 mg, or 300 mg of the at least 100 ABPs in a vial.

In typical embodiments, the pharmaceutical composition in the unit dosage form is in liquid form. In various embodiments, the unit dosage form contains between 0.1 mL and 50 ml of the active ingredient. In some embodiments, the unit dosage form contains 1 ml, 2.5 ml, 5 ml, 7.5 ml, 10 ml, 25 ml, or 50 ml of active ingredient.

In particular embodiments, the unit dosage form is a vial containing 1 ml of the pharmaceutical composition containing an active ingredient (e.g., ABP) at a concentration of 0.01 mg/ml, 0.1 mg/ml, 0.5 mg/ml, or 1 mg/ml. In particular embodiments, the unit dosage form is a vial containing 1 ml of the pharmaceutical composition containing an active ingredient (e.g., ABP) at a concentration of 0.01 mg/ml, 0.1 mg/ml, 0.5 mg/ml, 1 mg/ml, 2 mg/ml, 3 mg/ml, 4 mg/ml, 5 mg/ml, 10 mg/ml, 15 mg/ml, 20 mg/ml, 25 mg/ml, 30 mg/ml, 35 mg/ml, 40 mg/ml, 45 mg/ml or 50 mg/ml, 100 mg/ml, 250 mg/ml, 500 mg/ml, 750 mg/ml, 1 g/ml, 5 g/ml, 10 g/ml, or 50 g/ml. In some embodiments, the unit dosage form is a vial containing 2 ml of the pharmaceutical composition containing an active ingredient at a concentration of 0.01 mg/ml, 0.1 mg/ml, 0.5 mg/ml, 1 mg/ml, 2 mg/ml, 3 mg/ml, 4 mg/ml, 5 mg/ml, 10 mg/ml, 15 mg/ml, 20 mg/ml, 25 mg/ml, 30 mg/ml, 35 mg/ml, 40 mg/ml, 45 mg/ml or 50 mg/ml, 100 mg/ml, 250 mg/ml, 500 mg/ml, 750 mg/ml, 1 g/ml, 5 g/ml, 10 g/ml, or 50 g/ml.

In some embodiments, the pharmaceutical composition is formulated for injection of an active ingredient at a single dose or multiple doses of between 0.010 and 5 g/kg body weight. In some embodiments, the pharmaceutical composition is formulated for injection of an active ingredient at a single dose of 0.010 g/kg body weight. In some embodiments, the pharmaceutical composition is formulated for injection at a single dose of 0.01, 0.05, 0.15, 0.20, 0.25, 0.30, 0.35, 0.40, 0.45, 0.50, 0.55, 0.60, 0.65, 0.70, 0.75, 0.80, 0.85, 0.90, 0.95, or 0.1 g/kg body weight. In some embodiments, the pharmaceutical composition is formulated for injection at a single dose of 0.01, 0.05, 0.15, 0.20, 0.25, 0.30, 0.35, 0.40, 0.45, 0.50, 0.55, 0.60, 0.65, 0.70, 0.75, 0.80, 0.85, 0.90, 0.95, 0.1, 0.5, 1, 5, 10, 15, 20, 21, 25, 30, 35, 40, 45, 50, 55, 60, 65, 70, 75, 80, 85, 90, 95, 100, 105, 110, 115, 120, 125, 130, 135, 140, 145, 150, 175, 200, 225, 250, 275, 300, 325, 350, 375, 400, 425, 450, 475, 500, 525, 550, 575, 600, 625, 650, 675, 700, 725, 750, 775, or 800 mg/kg body weight. In some embodiments, the pharmaceutical composition is formulated for injection at a single dose of 0.01, 0.05, 0.15, 0.20, 0.25, 0.30, 0.35, 0.40, 0.45, 0.50, 0.55, 0.60, 0.65, 0.70, 0.75, 0.80, 0.85, 0.90, 0.95, 0.1, 0.5, 1, 5, 10, 15, 20, 21, 25, 30, 35, 40, 45, 50, 55, 60, 65, 70, 75, 80, 85, 90, 95, 100, 105, 110, 115, 120, 125, 130, 135, 140, 145, 150, 175, 200, 225, 250, 275, 300, 325, 350, 375, 400, 425, 450, 475, 500, 525, 550, 575, 600, 625, 650, 675, 700, 725, 750, 775, or 800 mg.

In some embodiments, the single dose is administered once. In some embodiments, the single dose is injected more than once.

In some embodiments, the unit dose contains at least 0.1 g, 0.5 g, 1 g, 1.5 g, 2 g, 2.5 g, 3 g, 3.5 g, 4 g, 5 g, 10 g, 20 g, 30 g, 40 g, or 50 g of the active ingredient (e.g., ABP). In some embodiments, the unit dose contains about 0.1 g, 0.5 g, 1 g, 1.5 g, 2 g, 2.5 g, 3 g, 3.5 g, 4 g, 5 g, 10 g, 20 g, 30 g, 40 g, or 50 g of the active ingredient (e.g., ABP). The amount of active ingredient which can be combined with a carrier material to produce a single dosage form will generally be that amount of the compound which produces a therapeutic effect.

The pharmaceutical compositions may conveniently be presented in unit dosage form.

The unit dosage form will typically be adapted to one or more specific routes of administration of the pharmaceutical composition.

In some embodiments, the pharmaceutical composition in the unit dosage form is in solid form, such as a lyophilate, suitable for solubilization.

In some embodiments, the unit dosage form is suitable for subcutaneous, intradermal, or intramuscular administration include preloaded syringes, auto-injectors, and auto-inject pens, each containing a predetermined amount of the pharmaceutical composition described hereinabove.

In various embodiments, the unit dosage form is a preloaded syringe, comprising a syringe and a predetermined amount of the pharmaceutical composition. In certain preloaded syringe embodiments, the syringe is adapted for subcutaneous administration. In certain embodiments, the syringe is suitable for self-administration. In particular embodiments, the preloaded syringe is a single use syringe.

In certain embodiments, the unit dosage form is an auto-inject pen. The auto-inject pen comprises an auto-inject pen containing a pharmaceutical composition as described herein. In some embodiments, the auto-inject pen delivers a predetermined volume of pharmaceutical composition. In other embodiments, the auto-inject pen is configured to deliver a volume of pharmaceutical composition set by the user.

Mixtures of Plasma IVIg with Recombinant Hyperimmunes

In some embodiments, a recombinant hyperimmune is spiked into conventional plasma IVIg to increase the anti-pathogen titer of IVIg. In some embodiments, several anti-pathogen recombinant hyperimmunes are spiked into conventional plasma IVIg. Any number of spike-ins can be mixed with plasma IVIg to generate increased titers against any number of pathogens.

In some embodiments, the spike-in recombinant hyperimmunes are mixed with plasma IVIg by the pharmacist. In some embodiments, the spike-in recombinant hyperimmunes are mixed with plasma IVIg by the manufacturer.

### 7.4. Polynucleotide Constructs

In one aspect, the present invention provides isolated polynucleotide constructs. In some embodiments, the human recombinant antibodies disclosed herein are generated from polynucleotide constructs encoding a cognate pair of heavy chain and light chain variable regions. In some embodiments, the polynucleotide construct further encodes a kappa or lambda-type light chain constant region, and a heavy chain IgG constant region.

In some embodiments, the polynucleotide construct encodes all or part of a recombinant antibody, for example, one or both chains of an antibody of the invention, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing. The polynucleotide construct can be any length. They can be, for example, 5, 10, 15, 20, 25, 30, 35, 40, 45, 50, 75, 100, 125, 150, 175, 200, 250, 300, 350, 400, 450, 500, 750, 1,000, 1,500, 3,000, 5,000 or more nucleotides in length, and/or can comprise one or more additional sequences, for example, regulatory sequences, and/or be part of a larger nucleic acid, for example, a vector. The polynucleotide construct can be single-stranded or double-stranded and can comprise RNA and/or DNA nucleotides, and artificial variants thereof (e.g., peptide nucleic acids).

In some embodiments, the polynucleotide construct has been enriched based on binding affinity of a protein encoded by the polynucleotide construct to a HBV antigen. In some embodiments, the polynucleotide construct has been enriched based on binding affinity of a protein encoded by the polynucleotide construct to a HBV surface antigen.

In some embodiments, the polynucleotide construct has been enriched based on neutralizing activity of a protein encoded by the polynucleotide construct to HBV. In some embodiments, the neutralizing activity is measured by detecting neutralization of live HBV infection to human hepatocyte cells.

Polynucleotides encoding antibody polypeptides (e.g., heavy or light chain, variable domain only, CDRs only, or full length) can be isolated from B cells, plasma cells, or plasmablasts of a subject that has been exposed to an antigen, e.g., by being infected by virus or immunized with a vaccine. The polynucleotide construct can be isolated by conventional procedures such as polymerase chain reaction (PCR) or methods described herein (e.g., single cell OE-RT-PCR).

Polypeptide sequences of the CDR3 from the variable regions of the heavy and light chain variable regions are shown herein. The skilled artisan will appreciate that, due to the degeneracy of the genetic code, each of the polypeptide sequences disclosed herein is encoded by a large number of other nucleic acid sequences. The present invention provides each degenerate nucleotide sequence encoding each antibody of the invention.

Methods for hybridizing nucleic acids are well-known in the art. See, e.g., Curr. Prot. in Mol. Biol., John Wiley & Sons, N.Y. (1989), 6.3.1-6.3.6. As defined herein, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C., in 0.5×SSC, 0.1% SDS. A stringent hybridization condition hybridizes in 6×SSC at 450 C, followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequences that are at least 65, 70, 75, 80, 85, 90, 95, 98, or 99% identical to each other typically remain hybridized to each other. The basic parameters affecting the choice of hybridization conditions and guidance for devising suitable conditions are set forth by, for example, Sambrook, Fritsch, and Maniatis (1989, Molecular Cloning: A Laboratory Manual, Cold Spring Harbor Laboratory Press, Cold Spring Harbor, N.Y., chapters 9 and 11; and Curr. Prot. in Mol. Biol. 1995, Ausubel et al., eds., John Wiley & Sons, Inc., sections 2.10 and 6.3-6.4), and can be readily determined by those having ordinary skill in the art based on, for example, the length and/or base composition of the DNA.

Changes can be introduced by mutation into a nucleic acid, thereby leading to changes in the amino acid sequence of a polypeptide (e.g., an antibody) that it encodes. Mutations can be introduced using any technique known in the art. In one embodiment, one or more particular amino acid residues are changed using, for example, a site-directed mutagenesis protocol. In another embodiment, one or more randomly selected residues are changed using, for example, a random mutagenesis protocol. However, it is made, a mutant polypeptide can be expressed and screened for a desired property (e.g., binding to a virus).

In another aspect, the present invention provides nucleic acid molecules that are suitable for use as primers or hybridization probes for the detection of nucleic acid sequences of the invention. A nucleic acid molecule of the invention can comprise only a portion of a nucleic acid sequence encoding a full-length polypeptide of the invention, for example, a fragment that can be used as a probe or primer or a fragment encoding an active portion (e.g., a virus binding portion) of a polypeptide of the invention.

Probes based on the sequence of a nucleic acid of the invention can be used to detect the nucleic acid or similar nucleic acids, for example, transcripts encoding a polypeptide of the invention. The probe can comprise a label group, e.g., a radioisotope, a fluorescent compound, an enzyme, or an enzyme co-factor. Such probes can be used to identify a cell that expresses the polypeptide

In another aspect, the present invention provides libraries of nucleic acids that encode for recombinant polyclonal antibodies or a variant or derivative thereof, derived from B cells, plasmablasts, and plasma cells. These libraries of nucleic acids are generated by isolating plasmablasts and plasma cells into single-cell reaction containers, wherein they are lysed and antibody-specific nucleic acids are purified or captured, for example on solid supports such as beads.

In some embodiments, the library is generated by obtaining a plurality of first linear polynucleotides, each comprising a first sequence encoding a heavy chain variable domain from a cognate pair from the single plasma cell or plasmablast; and a second sequence encoding a light chain variable domain from the cognate pair; and a third sequence linking the first and second sequences and comprising a restriction site; and obtaining a second linear polynucleotide, not operationally linked to the first polynucleotide, comprising a fourth sequence homologous to a portion of the first polynucleotide; and circularizing each of the plurality of first polynucleotides with the second polynucleotide to generate a library of polynucleotides encoding the library of recombinant antibodies, wherein circularization is effected through Gibson Assembly; and expressing the library of recombinant antibodies in mammalian cells comprising the library of polynucleotides encoding the recombinant antibodies, thereby generating the library of recombinant antibodies.

The present invention provides methods for performing capture of transcripts from millions of single cells in parallel. Capture of transcripts is followed by amplification of nucleic acids that encode heavy and light chain immunoglobulins, and subsequent linkage of said nucleic acids into libraries of fused constructs that encode both heavy and light chain immunoglobulins. In such libraries the native pairing of heavy and light chain immunoglobulins, as originally found in the input B cells, plasmablasts, and plasma cells, is maintained. Such methods are performed in parallel on millions of single cells, such that the resulting library of fused heavy and light chain immunoglobulin nucleic acids comprises natively paired sequences for millions of single cells. Such methods are described elsewhere (Adler et al., Mabs 9, 1282-1996, 2017; WO2020/223573 which are incorporated by reference in its entirety herein).

### 7.5. Vectors and Host Cells

The present invention provides vectors, each vector comprising a polynucleotide construct encoding a polypeptide of the invention or a portion thereof. Examples of vectors include, but are not limited to, plasmids, viral vectors, non-episomal mammalian vectors and expression vectors, for example, recombinant expression vectors.

In another aspect of the present invention, expression vectors containing the nucleic acid molecules and polynucleotides of the present invention are also provided, and host cells transformed with such vectors, and methods of producing the polynucleotide constructs are also provided. The term “expression vector” refers to a plasmid, phage, virus or vector for expressing a polypeptide from a polynucleotide sequence. Vectors for the expression of the polypeptides contain at a minimum sequence required for vector propagation and for expression of the cloned insert. An expression vector comprises a transcriptional unit comprising an assembly of (1) a genetic element or elements having a regulatory role in gene expression, for example, promoters or enhancers, (2) a sequence that encodes polypeptides and proteins to be transcribed into mRNA and translated into protein, and (3) appropriate transcription initiation and termination sequences. These sequences may further include a selection marker. Vectors suitable for expression in host cells are readily available and the nucleic acid molecules are inserted into the vectors using standard recombinant DNA techniques. Such vectors can include promoters which function in specific cells or tissues, and viral vectors for the expression of polypeptides in targeted human or animal cells.

The recombinant expression vectors of the invention can comprise a nucleic acid of the invention in a form suitable for expression of the nucleic acid in a host cell. The recombinant expression vectors include one or more regulatory sequences, selected on the basis of the host cells to be used for expression, which is operably linked to the nucleic acid sequence to be expressed. Regulatory sequences include those that direct constitutive expression of a nucleotide sequence in many types of host cells (e.g., SV40 early gene enhancer, Rous sarcoma virus promoter and cytomegalovirus promoter), those that direct expression of the nucleotide sequence only in certain host cells (e.g., tissue-specific regulatory sequences, see Voss et al., 1986, Trends Biochem. Sci. 11:287, Maniatis et al., 1987, Science 236:1237, incorporated by reference herein in their entireties), and those that direct inducible expression of a nucleotide sequence in response to particular treatment or condition (e.g., the metallothionin promoter in mammalian cells and the tet-responsive and/or streptomycin responsive promoter in both prokaryotic and eukaryotic systems (see id.). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

The invention further provides methods of making polypeptides, e.g., recombinant polyclonal antibodies. A variety of other expression/host systems may be utilized. Vector DNA can be introduced into prokaryotic or eukaryotic systems via conventional transformation or transfection techniques. These systems include but are not limited to microorganisms such as bacteria (for example, E. coli) transformed with recombinant bacteriophage, plasmid or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti or pBR322 plasmid); or animal cell systems. Mammalian cells useful in recombinant protein production include but are not limited to VERO cells, HeLa cells, Chinese hamster ovary (CHO) cell lines, or their derivatives such as Veggie CHO and related cell lines which grow in serum-free media (see Rasmussen et al., 1998, Cytotechnology 28:31) or CHO strain DX-B11, which is deficient in DHFR (see Urlaub et al., 1980, Proc. Natl. Acad. Sci. USA 77:4216-20) COS cells such as the COS-7 line of monkey kidney cells (ATCC CRL 1651) (see Gluzman et al., 1981, Cell 23:175), W138, BHK, HepG2, 3T3 (ATCC CCL 163), RIN, MDCK, A549, PC12, K562, L cells, C127 cells, BHK (ATCC CRL 10) cell lines, the CV1/EBNA cell line derived from the African green monkey kidney cell line CV1 (ATCC CCL 70) (see McMahan et al., 1991, EMBO J. 10:2821), human embryonic kidney cells such as 293, 293 EBNA or MSR 293, human epidermal A431 cells, human Colo205 cells, other transformed primate cell lines, normal diploid cells, cell strains derived from in vitro culture of primary tissue, primary explants, HL-60, U937, HaK or Jurkat cells. Mammalian expression allows the production of secreted or soluble polypeptides which may be recovered from the growth medium.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Once such cells are transformed with vectors that contain selectable markers as well as the desired expression cassette, the cells can be allowed to grow in an enriched media before they are switched to selective media, for example. The selectable marker is designed to allow growth and recovery of cells that successfully express the introduced sequences. Resistant clumps of stably transformed cells can be proliferated using tissue culture techniques appropriate to the cell line employed. An overview of expression of recombinant proteins is found in Methods of Enzymology, v. 185, Goeddell, D.V., ed., Academic Press (1990). Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die), among other methods.

The transformed cells can be cultured under conditions that promote expression of the polypeptide, and the polypeptide can be recovered by conventional protein purification procedures (as defined above).

In some cases, such as in expression using prokaryotic systems, the expressed polypeptides of this invention may need to be “refolded” and oxidized into a proper tertiary structure and disulfide linkages generated in order to be biologically active. Refolding can be accomplished using a number of procedures well known in the art. Such methods include, for example, exposing the solubilized polypeptide to a pH usually above 7 in the presence of a chaotropic agent. The selection of chaotrope is similar to the choices used for inclusion body solubilization; however, a chaotrope is typically used at a lower concentration. Exemplary chaotropic agents are guanidine and urea. In most cases, the refolding/oxidation solution will also contain a reducing agent plus its oxidized form in a specific ratio to generate a particular redox potential which allows for disulfide shuffling to occur for the formation of cysteine bridges. Some commonly used redox couples include cysteine/cystamine, glutathione/dithiobisGSH, cupric chloride, dithiothreitol DTT/dithiane DTT, and 2-mercaptoethanol (bME)/dithio-bME. In many instances, a co-solvent may be used to increase the efficiency of the refolding. Commonly used cosolvents include glycerol, polyethylene glycol of various molecular weights, and arginine.

In addition, the polypeptides can be synthesized in solution or on a solid support in accordance with conventional techniques. Various automatic synthesizers are commercially available and can be used in accordance with known protocols. See, for example, Stewart and Young, Solid Phase Peptide Synthesis, 2d. Ed., Pierce Chemical Co. (1984); Tam et al., J Am Chem Soc, 105:6442, (1983); Merrifield, Science 232:341-347 (1986); Barany and Merrifield, The Peptides, Gross and Meienhofer, eds, Academic Press, New York, 1-284; Barany et al., Int J Pep Protein Res, 30:705-739 (1987).

The polypeptides and proteins of the present invention can be purified according to protein purification techniques well known to those of skill in the art. These techniques involve, at one level, the crude fractionation of the proteinaceous and non-proteinaceous fractions. Having separated the peptide polypeptides from other proteins, the peptide or polypeptide of interest can be further purified using chromatographic and electrophoretic techniques to achieve partial or complete purification (or purification to homogeneity). The term “purified polypeptide” as used herein, is intended to refer to a composition, isolatable from other components, wherein the polypeptide is purified to any degree relative to its naturally-obtainable state. A purified polypeptide therefore also refers to a polypeptide that is free from the environment in which it may naturally occur. Generally, “purified” will refer to a polypeptide composition that has been subjected to fractionation to remove various other components, and which composition substantially retains its expressed biological activity. Where the term “substantially purified” is used, this designation will refer to a peptide or polypeptide composition in which the polypeptide or peptide forms the major component of the composition, such as constituting about 50%, about 60%, about 70%, about 80%, about 85%, or about 90% or more of the proteins in the composition.

Various techniques suitable for use in purification are well known to those of skill in the art. These include, for example, precipitation with ammonium sulphate, PEG, antibodies (immunoprecipitation) and the like or by heat denaturation, followed by centrifugation; chromatography such as affinity chromatography (Protein-A columns), ion exchange, gel filtration, reverse phase, hydroxylapatite, hydrophobic interaction chromatography, isoelectric focusing, gel electrophoresis, and combinations of these techniques. As is generally known in the art, it is believed that the order of conducting the various purification steps may be changed, or that certain steps may be omitted, and still result in a suitable method for the preparation of a substantially purified polypeptide. Exemplary purification steps are provided in the Examples below.

Various methods for quantifying the degree of purification of polypeptide are known to those of skill in the art in light of the present disclosure. These include, for example, determining the specific binding activity of an active fraction, or assessing the amount of peptide or polypeptide within a fraction by SDS/PAGE analysis. A preferred method for assessing the purity of a polypeptide fraction is to calculate the binding activity of the fraction, to compare it to the binding activity of the initial extract, and to thus calculate the degree of purification, herein assessed by a “-fold purification number.” The actual units used to represent the amount of binding activity are, of course, be dependent upon the particular assay technique chosen to follow the purification and whether or not the polypeptide or peptide exhibits a detectable binding activity.

For example, in some embodiments, the purification process comprises one or more steps selected from: Clarification, Affinity Chromatography, Viral Inactivation and Depth Filtration, Hydrophobic Interaction Chromatography or Membrane Filtration, Multimodal Anion Exchange Chromatography or Membrane Filtration, Anion Exchange Chromatography or Membrane Filtration, Multimodal Cation Exchange Chromatography, Cation Exchange Chromatography, Virus Filtration and UF/DF. In some embodiments, protein concentration is measured between each of the steps

In some aspects, the present disclosure includes libraries of antibody-encoding nucleic acid vectors for site-directed integration into mammalian genomes. Such vectors include plasmids, retroviruses, and lentivirus. These libraries of vectors encode libraries of antibody sequences, which are then be used to engineer mammalian cells for production of antibodies. The libraries of nucleic acid vectors may include 10, 100, 1,000, 10,000, or more than 100,000 different antibody-encoding sequences. The sequences are derived from plasmablasts and plasma cells. These libraries of nucleic acids are generated by isolating plasmablasts and plasma cells into single-cell reaction containers, wherein they are lysed and antibody-specific nucleic acids are purified or captured, for example on solid supports such as beads. The present invention provides methods for performing capture of transcripts from millions of single cells in parallel. Capture of transcripts is followed by amplification of nucleic acids that encode heavy and light chain immunoglobulins, and subsequent linkage of said nucleic acids into libraries of fused constructs that encode both heavy and light chain immunoglobulins. In such libraries the native pairing of heavy and light chain immunoglobulins, as originally found in the input plasmablasts and plasma cells, is maintained. Such methods are performed in parallel on millions of single cells, such that the resulting library of fused heavy and light chain immunoglobulin nucleic acids comprises natively paired sequences for millions of single cells. These paired fused amplicons are then engineered into full-length antibody constructs using Gibson Assembly, restriction endonucleases, or other recombinant DNA techniques.

Engineering into full-length antibody constructs is performed on the full library en masse, such that the antibody sequence content and antibody sequence counts of the library are essentially maintained throughout the process. In some aspects, the library of expression vectors is engineered in two steps, such that the scFv amplicon is subcloned into an intermediate vector, and then a second round of Gibson Assembly, restriction digestion, or other recombinant technique is used to engineer additional domains of the antibody into the linker of the scFv. The method is described in U.S. Pat. No. 9,422,547, which is incorporated by reference in its entirety herein. The native pairing of heavy and light chain immunoglobulins is essentially maintained throughout the process of engineering into full-length expression vector libraries. The vectors are designed in various orientations, for example, two separate promoters drive expression of heavy and light chain immunoglobulins, or one promoter drives expression of both heavy and light chain immunoglobulins, and a translational skip motif is used to separately translate the heavy and light chain immunoglobulins into separate polypeptides. In some embodiments, the expression vectors comprise sequences for site-directed integration into mammalian production cells, for example, CRISPR-Cas9, Flp-In, Cre/Lox, or zinc finger recombination methods. Site-directed integration ensures that each mammalian production cell encodes a single antibody sequence, and decreases variability in expression levels between single production cells.

In another aspect, the present disclosure provides a host cell or a library of host cells, each comprising a polynucleotide encoding an antibody described herein. In some embodiments, the host cell comprises a vector or comprises a polynucleotide stably integrated into its genome. In some embodiments, the host cell comprises a polynucleotide encoding a ABP stably integrated into the genome using a Flp recombinase recognition target (FRT) landing pad or a similar method known in the art. In some embodiments, the host cell is a mammalian or prokaryotic cell. In some embodiments, the host cell is a human cell or a yeast cell. In some embodiments, the host cell is CHO cell.

In some embodiments, the host cell comprises an expression vector or integrated polynucleotide for production of the antibody. In some embodiments, a library of host cells can be used produce recombinant polyclonal antibodies described herein. In some embodiments, a library of host cells comprise cells selected based on their production yields or neutralization or binding titers of recombinant polyclonal antibodies produced by them. The host cells can be production host cells.

### 7.6. Methods of Producing Antibodies

The recombinant polyclonal antibodies can be purified from host cells that comprises a gene encoding the recombinant polyclonal antibodies. The recombinant polyclonal antibodies produced from the host cells can be obtained by elution of filtered supernatant of host cell culture fluid using a Heparin HP column, using a salt gradient, or with protein A resin.

Thus, in one aspect, the present disclosure provides a method of making recombinant antibodies for treatment of a patient exposed to HBV, comprising: isolating single cells from a blood sample from a donor exposed to a Hepatitis B virus (HBV) surface antigen; amplifying polynucleotides from the isolated single cells, wherein each polynucleotide encodes a cognate pair of heavy chain and light chain variable regions from a single cell by overlap extension reverse transcriptase polymerase chain reaction (OE-RT-PCR); cloning the polynucleotides obtained from the amplification into an expression vector, thereby obtaining polynucleotide constructs encoding the recombinant antibodies; generating recombinant antibodies from the polynucleotide constructs; and purifying the recombinant antibodies. In some embodiments, the recombinant antibodies are generated by the method described in PCT/US2016/033109, which is incorporated by reference in its entirety here.

In some embodiments, the method for preparing a plurality of recombinant immunoglobulin expression constructs, comprises: providing a plurality of recombinant fusion polynucleotides each comprising a first polynucleotide encoding a first variable domain, a second polynucleotide encoding a second variable domain, and a linker polynucleotide linking the first and second polynucleotides; expressing a plurality of recombinant fusion polypeptides encoded by said plurality of recombinant fusion polynucleotides to display said plurality of recombinant fusion polypeptides on a plurality of surfaces; enriching said plurality of recombinant fusion polynucleotides for binding to an antigen by exposing said plurality of surfaces to said antigen and selecting based on binding between said antigen and each of said plurality of recombinant fusion polypeptides; circularizing one or more of said enriched recombinant fusion polynucleotides; and inserting a third polynucleotide comprising a sequence encoding a promoter and a sequence encoding a constant region between said first and second polynucleotide in each of said circularized enriched recombinant fusion polynucleotides, thereby generating a plurality of recombinant immunoglobulin expression constructs.

In an embodiment, the method further comprises inserting said plurality of recombinant immunoglobulin expression constructs into a plurality of host cells; and expressing said plurality of recombinant immunoglobulin expression construct in said plurality of host cells, thereby generating a recombinant immunoglobulin library, wherein each recombinant immunoglobulin comprises a linked heavy chain and light chain variable domain cognate pair from a single isolated cell.

In an embodiment, the first variable domain is from an immunoglobulin heavy chain, and said second variable domain is from an immunoglobulin light chain. In an embodiment, the plurality of recombinant immunoglobulin expression constructs comprises at least 1,000, 10,000, or 100,000 unique cognate pairs of heavy chain and light chain encoding sequences.

Also provided herein is a method of generating an immunoglobulin library, comprising: providing a plurality of circularized polynucleotide constructs, each comprising a first polynucleotide, a second polynucleotide, and a linker polynucleotide linking the first and second polynucleotides, wherein said first polynucleotide comprises a region encoding a first variable domain from a single isolated cell, wherein said second polynucleotide comprises a region encoding a second variable domain from said single isolated cell, and wherein each of said plurality of circularized polynucleotide constructs comprises a cognate pair of linked first and second variable domains from a single isolated cell; inserting a third polynucleotide comprising a sequence encoding a promoter and a sequence encoding a constant region between said first and second polynucleotide in each of said circularized recombinant fusion polynucleotides, thereby generating a plurality of recombinant immunoglobulin expression construct; inserting said plurality of recombinant immunoglobulin expression constructs into a plurality of host cells; and expressing said plurality of recombinant immunoglobulin expression construct in said plurality of host cells, thereby generating a recombinant immunoglobulin library comprising a plurality of recombinant immunoglobulins, wherein each of said plurality of recombinant immunoglobulins comprises a linked heavy chain variable domain and light chain variable domain cognate pair from a single isolated cell.

In an embodiment, the insertion of said third polynucleotide is performed in parallel for said plurality of circularized polynucleotide constructs. In an embodiment, the first variable domain is from an immunoglobulin heavy chain, and said second variable domain is from an immunoglobulin light chain. In an embodiment, the plurality of recombinant fusion protein expression constructs are expression vectors. In an embodiment, the insertion of said third polynucleotide comprises a method selected from the group consisting of. Gibson assembly, site-specific digestion and ligation, and targeted recombination.

In an embodiment, the first and second nucleic acid probes are bound to a particle. In an embodiment, the particle is a bead comprising agarose, glass, chemical polymers, or magnetic materials. In an embodiment, the probes comprise biotin and wherein said particle comprises streptavidin bound to the surface of the particle. In an embodiment, the composition comprises a covalent bond between said first probe or said second probe and said particle. In an embodiment, each of said plurality of monodisperse aqueous droplets further comprise reagents for overlap extension RT-PCR. In an embodiment, the first probe is bound to a polynucleotide encoding a light chain variable domain from a single isolated cell, and wherein said second probe is bound to a polynucleotide encoding a heavy chain variable domain from said single isolated cell.

In an embodiment, the first and second variable domains are from a single cell. In an embodiment, the first variable domain is from an immunoglobulin heavy chain, and said second variable domain is from an immunoglobulin light chain. In an embodiment, the recombinant fusion protein expression construct is an expression vector.

Also provided herein is a method for identifying antibodies of interest, comprising providing primary immune cells from at least one mammalian donor; isolating single immune cells or subpopulations of immune cells from said primary immune cells in a reaction vessel; generating a plurality of recombinant fusion polynucleotides each comprising a first polynucleotide encoding a light chain variable domain polypeptide and a second polynucleotide encoding a heavy chain variable domain polypeptide, wherein said first and second polynucleotides are each from said immune cells or subpopulations of immune cells, wherein said recombinant fusion polynucleotides further comprise a linker polynucleotide linking said first and second polynucleotides; inserting at least one of each of said plurality of recombinant fusion polynucleotides into a plurality of expression vectors; expressing said expression vectors in a host cell to generate a plurality of recombinant immunoglobulins; and identifying therapeutic antibodies from said plurality of recombinant immunoglobulins that bind to an antigen of interest.

In some embodiments, the method further comprises enriching the recombinant antibodies based on binding affinity of the recombinant antibodies to the HBV surface antigen. In some embodiments, the method further comprises enriching the recombinant antibodies based on neutralizing activity of the recombinant antibodies to live HBV.

Fully human monoclonal antibodies can be generated by any number of techniques with which those having ordinary skill in the art are familiar. Such methods include, but are not limited to, Epstein Barr Virus (EBV) transformation of human peripheral blood cells (e.g., containing B lymphocytes), in vitro immunization of human B-cells, fusion of spleen cells from immunized transgenic mice carrying inserted human immunoglobulin genes, isolation from human immunoglobulin V region phage libraries, or other procedures as known in the art and based on the disclosure herein. For example, fully human monoclonal antibodies may be obtained from transgenic mice that have been engineered to produce specific human antibodies in response to antigenic challenge. Methods for obtaining fully human antibodies from transgenic mice are described, for example, by Green et al., Nature Genet. 7:13, 1994; Lonberg et al., Nature 368:856, 1994; Taylor et al., Int. Immun. 6:579, 1994; U.S. Pat. No. 5,877,397; Bruggemann et al., 1997 Curr. Opin. Biotechnol. 8:455-58; Jakobovits et al., 1995 Ann. N. Y. Acad. Sci. 764:525-35. In this technique, elements of the human heavy and light chain locus are introduced into strains of mice derived from embryonic stem cell lines that contain targeted disruptions of the endogenous heavy chain and light chain loci (see also Bruggemann et al., Curr. Opin. Biotechnol. 8:455-58 (1997)). For example, human immunoglobulin transgenes may be mini-gene constructs, or transloci on yeast artificial chromosomes, which undergo B-cell-specific DNA rearrangement and hypermutation in the mouse lymphoid tissue. Fully human monoclonal antibodies may be obtained by immunizing the transgenic mice, which may then produce human antibodies specific for the antigen target or targets. Lymphoid cells of the immunized transgenic mice can be used to produce human antibody-secreting hybridomas according to the methods described herein.

Another method for generating human antibodies of the invention includes immortalizing human peripheral blood cells by EBV transformation. See, e.g., U.S. Pat. No. 4,464,456. Such an immortalized B-cell line (or lymphoblastoid cell line) producing an antibody that specifically binds to target or targets can be identified by immunodetection methods as provided herein, for example, an ELISA, and then isolated by standard cloning techniques. The stability of the lymphoblastoid cell line producing an antibody can be improved by fusing the transformed cell lines with a murine myeloma to produce a mouse-human hybrid cell line according to methods known in the art (see, e.g., Glasky et al., Hybridoma 8:377-89 (1989)). Still another method to generate human antibodies is in vitro immunization, which includes priming human splenic B-cells with antigen targets, followed by fusion of primed with a hetero-hybrid fusion partner. See, e.g., Boerner et al., 1991 J. Immunol. 147:86-95.

In certain embodiments, B-cells that are producing recombinant polyclonal antibodies are selected and the light chain and heavy chain variable regions are cloned from the B-cell according to molecular biology techniques known in the art (WO 92/02551; U.S. Pat. No. 5,627,052; Babcook et al., Proc. Natl. Acad. Sci. USA 93:7843-48 (1996)) and described herein. B-cells from an immunized animal may be isolated from the spleen, lymph node, or peripheral blood sample by selecting a cell that is producing an antibody that specifically binds to the antigen target. B-cells may also be isolated from humans, for example, from a peripheral blood sample.

Methods for detecting single B-cells that are producing an antibody with the desired specificity are well known in the art, for example, by plaque formation, fluorescence-activated cell sorting, in vitro stimulation followed by detection of specific antibody, and the like. Methods for selection of specific antibody-producing B-cells include, for example, preparing a single cell suspension of B-cells in soft agar that contains the antigen target. Binding of the specific antibodies produced by the B-cell to the antigen results in the formation of a complex, which may be visible as an immunoprecipitate.

In some embodiments, specific antibody-producing B-cells are selected by using a method that allows identification natively paired antibodies. For example, a method described in Adler et al., A natively paired antibody library yields drug leads with higher sensitivity and specificity than a randomly paired antibody library, MAbs (2018), which is incorporated by reference in its entirety herein, can be employed. The method combines microfluidic technology, molecular genomics, yeast single-chain variable fragment (scFv) display, fluorescence-activated cell sorting (FACS) and deep sequencing. In short, B cells can be isolated from immunized animals and then pooled. The B cells are encapsulated into droplets with oligo-dT beads and a lysis solution, and mRNA-bound beads are purified from the droplets, and then injected into a second emulsion with an OE-RT-PCR amplification mix that generates DNA amplicons that encode scFv with native pairing of heavy and light chain 1 g. Libraries of natively paired amplicons are then electroporated into yeast for scFv display. FACS is used to identify high affinity scFv. Finally, deep antibody sequencing can be used to identify all clones in the pre- and post-sort scFv libraries.

After the B-cells producing the desired antibodies are selected, the specific antibody genes may be cloned by isolating and amplifying DNA or mRNA according to methods known in the art and described herein.

The methods for obtaining antibodies of the invention can also adopt various phage display technologies known in the art. See, e.g., Winter et al., 1994 Annu. Rev. Immunol. 12:433-55; Burton et al., 1994 Adv. Immunol. 57:191-280. Human or murine immunoglobulin variable region gene combinatorial libraries may be created in phage vectors that can be screened to select Ig fragments (Fab, Fv, sFv, or multimers thereof) that bind specifically to the recombinant polyclonal antibodies or variant or fragment thereof. See, e.g., U.S. Pat. No. 5,223,409; Huse et al., 1989 Science 246:1275-81; Sastry et al., Proc. Natl. Acad. Sci. USA 86:5728-32 (1989); Alting-Mees et al., Strategies in Molecular Biology 3:1-9 (1990); Kang et al., 1991 Proc. Natl. Acad. Sci. USA 88:4363-66; Hoogenboom et al., 1992 J. Molec. Biol. 227:381-388; Schlebusch et al., 1997 Hybridoma 16:47-52 and references cited therein. For example, a library containing a plurality of polynucleotide sequences encoding Ig variable region fragments may be inserted into the genome of a filamentous bacteriophage, such as M13 or a variant thereof, in frame with the sequence encoding a phage coat protein. A fusion protein may be a fusion of the coat protein with the light chain variable region domain and/or with the heavy chain variable region domain. According to certain embodiments, immunoglobulin Fab fragments may also be displayed on a phage particle (see, e.g., U.S. Pat. No. 5,698,426).

In one embodiment, in a hybridoma the variable regions of a gene expressing a monoclonal antibody of interest are amplified using nucleotide primers. These primers may be synthesized by one of ordinary skill in the art or may be purchased from commercially available sources. (See, e.g., Stratagene (La Jolla, California), which sells primers for mouse and human variable regions including, among others, primers for V_{Ha}, V_{Hb}, V_{Hc}, V_{Hd}, C_{H1}, V_{L} and C_{L} regions.) These primers may be used to amplify heavy or light chain variable regions, which may then be inserted into vectors such as ImmunoZAP™H or ImmunoZAP™L (Stratagene), respectively. These vectors may then be introduced into E. coli, yeast, or mammalian-based systems for expression. Large amounts of a single-chain protein containing a fusion of the V_{H} and V_{L} domains may be produced using these methods (see Bird et al., Science 242:423-426, 1988).

Once cells producing antibodies according to the invention have been obtained using any of the above-described immunization and other techniques, the specific antibody genes may be cloned by isolating and amplifying DNA or mRNA therefrom according to standard procedures as described herein. The antibodies produced therefrom may be sequenced and the CDRs identified and the DNA coding for the CDRs may be manipulated as described previously to generate other antibodies according to the invention.

The recombinant polyclonal antibodies of the present invention preferably have activity in the cell-based assays described herein and/or the in vivo assay described herein and/or bind to one or more of the antigens described herein. Accordingly, such binding agents can be identified using the assays described herein.

Other antibodies according to the invention may be obtained by conventional immunization and cell fusion procedures as described herein and known in the art.

Molecular evolution of the complementarity determining regions (CDRs) in the center of the antibody binding site also has been used to isolate antibodies with increased affinity, for example, antibodies having increased affinity for c-erbB-2, as described by Schier et al., 1996, J Mol. Biol. 263:551.

Human, partially human, or humanized antibodies are suitable for many applications, particularly those involving administration of the antibody to a human subject, other types of antigen binding proteins. The non-human antibodies of the invention can be, for example, derived from any antibody-producing animal, such as mouse, rat, rabbit, goat, donkey, or non-human primate (such as monkey (e.g., cynomologous or rhesus monkey) or ape (e.g., chimpanzee)). Non-human antibodies of the invention can be used, for example, in in vitro and cell-culture based applications, or any other application where an immune response to the antibody of the invention does not occur, is insignificant, can be prevented, is not a concern, or is desired. In one embodiment, a non-human antibody of the invention is administered to a non-human subject. In another embodiment, the non-human antibody does not elicit an immune response in the non-human subject. In another embodiment, the non-human antibody is from the same species as the non-human subject, e.g., a mouse antibody of the invention is administered to a mouse. An antibody from a particular species can be made by, for example, immunizing an animal of that species with the desired immunogen or using an artificial system for generating antibodies of that species (e.g., a bacterial or phage display-based system for generating antibodies of a particular species), or by converting an antibody from one species into an antibody from another species by replacing, e.g., the constant region of the antibody with a constant region from the other species, or by replacing one or more amino acid residues of the antibody so that it more closely resembles the sequence of an antibody from the other species. In one embodiment, the antibody is a chimeric antibody comprising amino acid sequences derived from antibodies from two or more different species.

The recombinant polyclonal antibodies may be prepared, and screened for desired properties, by any of a number of conventional techniques. Certain of the techniques involve isolating a nucleic acid encoding a polypeptide chain (or portion thereof) of an antibody of interest, and manipulating the nucleic acid through recombinant DNA technology. The nucleic acid may be fused to another nucleic acid of interest, or altered (e.g., by mutagenesis or other conventional techniques) to add, delete, or substitute one or more amino acid residues, for example. Furthermore, the antigen binding proteins may be purified from cells that naturally express them (e.g., an antibody can be purified from a hybridoma that produces it), or produced in recombinant expression systems, using any technique known in the art. See, for example, Monoclonal Antibodies, Hybridomas: A New Dimension in Biological Analyses, Kennet et al. (eds.), Plenum Press, New York (1980); and Antibodies: A Laboratory Manual, Harlow and Land (eds.), Cold Spring Harbor Laboratory Press, Cold Spring Harbor, NY, (1988).

Any expression system known in the art can be used to make the recombinant polypeptides of the invention. Expression systems are detailed comprehensively above. In general, host cells are transformed with a recombinant expression vector that comprises DNA encoding a desired polypeptide. Among the host cells that may be employed are prokaryotes, yeast or higher eukaryotic cells. Prokaryotes include gram negative or gram-positive organisms, for example E. coli or Bacilli. Higher eukaryotic cells include insect cells and established cell lines of mammalian origin. Examples of suitable mammalian host cell lines include the COS-7 line of monkey kidney cells (ATCC CRL 1651) (Gluzman et al., 1981, Cell 23:175), L cells, 293 cells, C127 cells, 3T3 cells (ATCC CCL 163), Chinese hamster ovary (CHO) cells, HeLa cells, BHK (ATCC CRL 10) cell lines, and the CVI/EBNA cell line derived from the African green monkey kidney cell line CVI (ATCC CCL 70) as described by McMahan et al., 1991, EMBO J. 10: 2821. Appropriate cloning and expression vectors for use with bacterial, fungal, yeast, and mammalian cellular hosts are described by Pouwels et al. (Cloning Vectors: A Laboratory Manual, Elsevier, New York, 1985).

Production cell lines for monoclonal antibodies (mAbs) are typically produced by randomly inserting expression constructs into a mammalian production cell genome, for example, a CHO genome (Rita Costa et al., 2010). However, this canonical method produces cell lines with multiple copies of mAb inserted into the CHO genome. If the polyclonal antibody construct libraries were randomly inserted into the CHO genome, many clones would express multiple antibodies, which would result in frequent non-native pairing between heavy and light chain Ig. Additionally, different genome locations have different transcriptional activity levels (Kito et al., 2002), which could result in heterogeneous, inconsistent and/or unstable bioproduction. Thus, in some aspects, the current invention provides a CHO cell line with a Flp recombinase recognition target (FRT) landing pad stably engineered into the genome. Such site-directed genome integration cell lines are then used for stable expression of recombinant polyclonal antibodies.

It will be appreciated that an antibody of the present invention may have at least one amino acid substitution, providing that the antibody retains binding specificity. Therefore, modifications to the antibody structures are encompassed within the scope of the invention. These may include amino acid substitutions, which may be conservative or non-conservative that do not destroy the binding capability of an antibody comprising the recombinant polyclonal antibodies. Conservative amino acid substitutions may encompass non-naturally occurring amino acid residues, which are typically incorporated by chemical peptide synthesis rather than by synthesis in biological systems. These include peptidomimetics and other reversed or inverted forms of amino acid moieties. A conservative amino acid substitution may also involve a substitution of a native amino acid residue with a normative residue such that there is little or no effect on the polarity or charge of the amino acid residue at that position.

Non-conservative substitutions can involve the exchange of a member of one class of amino acids or amino acid mimetics for a member from another class with different physical properties (e.g. size, polarity, hydrophobicity, charge). Such substituted residues can be introduced into regions of the human antibody that are homologous with non-human antibodies, or into the non-homologous regions of the molecule.

Moreover, one skilled in the art may generate test variants containing a single amino acid substitution at each desired amino acid residue. The variants can then be screened using activity assays known to those skilled in the art. Such variants could be used to gather information about suitable variants. For example, if one discovered that a change to a particular amino acid residue resulted in destroyed, undesirably reduced, or unsuitable activity, variants with such a change may be avoided. In other words, based on information gathered from such routine experiments, one skilled in the art can readily determine the amino acids where further substitutions should be avoided either alone or in combination with other mutations.

A skilled artisan will be able to determine suitable variants of the polypeptide as set forth herein using well-known techniques. In certain embodiments, one skilled in the art may identify suitable areas of the molecule that may be changed without destroying activity by targeting regions not believed to be important for activity. In certain embodiments, one can identify residues and portions of the molecules that are conserved among similar polypeptides. In certain embodiments, even areas that may be important for biological activity or for structure may be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, one can predict the importance of amino acid residues in a protein that correspond to amino acid residues which are important for activity or structure in similar proteins. One skilled in the art can opt for chemically similar amino acid substitutions for such predicted important amino acid residues.

One skilled in the art can also analyze the three-dimensional structure and amino acid sequence in relation to that structure in similar polypeptides. In view of such information, one skilled in the art may predict the alignment of amino acid residues of an antibody with respect to its three-dimensional structure. In certain embodiments, one skilled in the art can choose not to make radical changes to amino acid residues predicted to be on the surface of the protein, since such residues may be involved in important interactions with other molecules.

A number of scientific publications have been devoted to the prediction of secondary structure. See Moult J, Curr. Op. in Biotech., 7(4):422-427 (1996), Chou et al., Biochem., 13(2):222-245 (1974); Chou et al., Biochem., 113(2):211-222 (1974); Chou et al., Adv. Enzymol. Relat. Areas Mol. Biol., 47:45-148 (1978); Chou et al., Ann. Rev. Biochem., 47:251-276 and Chou et al., Biophys. J., 26:367-384 (1979). Moreover, computer programs are currently available to assist with predicting secondary structure. One method of predicting secondary structure is based upon homology modeling. For example, two polypeptides or proteins which have a sequence identity of greater than 30%, or similarity greater than 40% often have similar structural topologies. The recent growth of the protein structural database (PDB) has provided enhanced predictability of secondary structure, including the potential number of folds within a polypeptide's or protein's structure. See Holm et al., Nucl. Acid. Res., 27(1):244-247 (1999). It has been suggested (Brenner et al., Curr. Op. Struct. Biol., 7(3):369-376 (1997)) that there are a limited number of folds in a given polypeptide or protein and that once a critical number of structures have been resolved, structural prediction will become dramatically more accurate.

Additional methods of predicting secondary structure include “threading” (Jones, D., Curr. Opin. Struct. Biol., 7(3):377-87 (1997); Sippl et al., Structure, 4(1):15-19 (1996)), “profile analysis” (Bowie et al., Science, 253:164-170 (1991); Gribskov et al., Meth. Enzym., 183:146-159 (1990); Gribskov et al., Proc. Nat. Acad. Sci., 84(13):4355-4358 (1987)), and “evolutionary linkage” (See Holm, supra (1999), and Brenner, supra (1997)).

In certain embodiments, variants of antibodies include glycosylation variants wherein the number and/or type of glycosylation site has been altered compared to the amino acid sequences of a parent polypeptide. In certain embodiments, variants comprise a greater or a lesser number of N-linked glycosylation sites than the native protein. An N-linked glycosylation site is characterized by the sequence: Asn-X-Ser or Asn-X-Thr, wherein the amino acid residue designated as X can be any amino acid residue except proline. The substitution of amino acid residues to create this sequence provides a potential new site for the addition of an N-linked carbohydrate chain. Alternatively, substitutions which eliminate this sequence will remove an existing N-linked carbohydrate chain. Also provided is a rearrangement of N-linked carbohydrate chains wherein one or more N-linked glycosylation sites (typically those that are naturally occurring) are eliminated and one or more new N-linked sites are created. Additional preferred antibody variants include cysteine variants wherein one or more cysteine residues are deleted from or substituted for another amino acid (e.g., serine) as compared to the parent amino acid sequence. Cysteine variants can be useful when antibodies must be refolded into a biologically active conformation such as after the isolation of insoluble inclusion bodies. Cysteine variants generally have fewer cysteine residues than the native protein, and typically have an even number to minimize interactions resulting from unpaired cysteines.

According to certain embodiments, preferred amino acid substitutions are those which: (1) reduce susceptibility to proteolysis, (2) reduce susceptibility to oxidation, (3) alter binding affinity for forming protein complexes, (4) alter binding affinities, and/or (4) confer or modify other physiochemical or functional properties on such polypeptides. According to certain embodiments, single or multiple amino acid substitutions (in certain embodiments, conservative amino acid substitutions) can be made in the naturally-occurring sequence (in certain embodiments, in the portion of the polypeptide outside the domain(s) forming intermolecular contacts). In certain embodiments, a conservative amino acid substitution typically may not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991), which are each incorporated herein by reference.

In certain embodiments, the recombinant polyclonal antibodies of the invention can be chemically bonded with polymers, lipids, or other moieties.

The binding agents may comprise at least one of the CDRs described herein incorporated into a biocompatible framework structure. In one example, the biocompatible framework structure comprises a polypeptide or portion thereof that is sufficient to form a conformationally stable structural support, or framework, or scaffold, which is able to display one or more sequences of amino acids that bind to an antigen (e.g., CDRs, a variable region, etc.) in a localized surface region. Such structures can be a naturally occurring polypeptide or polypeptide “fold” (a structural motif), or can have one or more modifications, such as additions, deletions or substitutions of amino acids, relative to a naturally occurring polypeptide or fold. These scaffolds can be derived from a polypeptide of any species (or of more than one species), such as a human, other mammal, other vertebrate, invertebrate, plant, bacteria or virus.

Typically, the biocompatible framework structures are based on protein scaffolds or skeletons other than immunoglobulin domains. For example, those based on fibronectin, ankyrin, lipocalin, neocarzinostain, cytochrome b, CP1 zinc finger, PST1, coiled coil, LACI-D1, Z domain and tendamistat domains may be used (See e.g., Nygren and Uhlen, 1997, Curr. Opin. in Struct. Biol., 7, 463-469).

It will be appreciated that the recombinant polyclonal antibodies of the invention include the humanized antibodies described herein. Humanized antibodies such as those described herein can be produced using techniques known to those skilled in the art (Zhang, W., et al., Molecular Immunology. 42(12):1445-1451, 2005; Hwang W. et al., Methods. 36(1):35-42, 2005; Dall'AcquaWF, et al., Methods 36(1):43-60, 2005; and Clark, M., Immunology Today. 21(8):397-402, 2000).

Where an antibody comprises one or more of CDR3-H, and/or CDR3-L, it may be obtained by expression from a host cell containing DNA coding for these sequences. A DNA coding for each CDR sequence may be determined on the basis of the amino acid sequence of the CDR and synthesized together with any desired antibody variable region framework and constant region DNA sequences using oligonucleotide synthesis techniques, site-directed mutagenesis and polymerase chain reaction (PCR) techniques as appropriate. DNA coding for variable region frameworks and constant regions is widely available to those skilled in the art from genetic sequences databases such as GenBank®.

Once synthesized, the DNA encoding an antibody of the invention or fragment thereof may be propagated and expressed according to any of a variety of well-known procedures for nucleic acid excision, ligation, transformation, and transfection using any number of known expression vectors. Thus, in certain embodiments expression of an antibody fragment may be preferred in a prokaryotic host, such as Escherichia coli (see, e.g., Pluckthun et al., 1989 Methods Enzymol. 178:497-515). In certain other embodiments, expression of the antibody or a fragment thereof may be preferred in a eukaryotic host cell, including yeast (e.g., Saccharomyces cerevisiae, Schizosaccharomyces pombe, and Pichia pastoris), animal cells (including mammalian cells) or plant cells. Examples of suitable animal cells include, but are not limited to, myeloma (such as a mouse NSO line), COS, CHO, or hybridoma cells. Examples of plant cells include tobacco, corn, soybean, and rice cells.

Replicable expression vectors containing DNA encoding an antibody variable and/or constant region may be prepared and used to transform an appropriate cell line, for example, a non-producing myeloma cell line, such as a mouse NSO line or a bacteria, such as E. co/i, in which production of the antibody will occur. In order to obtain efficient transcription and translation, the DNA sequence in each vector should include appropriate regulatory sequences, particularly a promoter and leader sequence operatively linked to the variable domain sequence. Particular methods for producing antibodies in this way are generally well-known and routinely used. For example, basic molecular biology procedures are described by Maniatis et al. (Molecular Cloning, A Laboratory Manual, 2nd ed., Cold Spring Harbor Laboratory, New York, 1989; see also Maniatis et al, 3rd ed., Cold Spring Harbor Laboratory, New York, (2001)). DNA sequencing can be performed as described in Sanger et al. (PNAS 74:5463, (1977)) and the Amersham International plc sequencing handbook, and site directed mutagenesis can be carried out according to methods known in the art (Kramer et al., Nucleic Acids Res. 12:9441, (1984); Kunkel Proc. Natl. Acad. Sci. USA 82:488-92 (1985); Kunkel et al., Methods in Enzymol. 154:367-82 (1987); the Anglian Biotechnology Ltd. handbook). Additionally, numerous publications describe techniques suitable for the preparation of antibodies by manipulation of DNA, creation of expression vectors, and transformation and culture of appropriate cells (Mountain A and Adair, J R in Biotechnology and Genetic Engineering Reviews (ed. Tombs, M P, 10, Chapter 1, 1992, Intercept, Andover, UK); “Current Protocols in Molecular Biology”, 1999, F. M. Ausubel (ed.), Wiley Interscience, New York).

Where it is desired to improve the affinity of antibodies according to the invention containing one or more of the above-mentioned CDRs can be obtained by a number of affinity maturation protocols including maintaining the CDRs (Yang et al., J. Mol. Biol., 254, 392-403, 1995), chain shuffling (Marks et al., Bio/Technology, 10, 779-783, 1992), use of mutation strains of E. coli. (Low et al., J. Mol. Biol., 250, 350-368, 1996), DNA shuffling (Patten et al., Curr. Opin. Biotechnol., 8, 724-733, 1997), phage display (Thompson et al., J. Mol. Biol., 256, 7-88, 1996) and sexual PCR (Crameri, et al., Nature, 391, 288-291, 1998). All of these methods of affinity maturation are discussed by Vaughan et al. (Nature Biotech., 16, 535-539, 1998).

It will be understood by one skilled in the art that some proteins, such as antibodies, may undergo a variety of posttranslational modifications. The type and extent of these modifications often depends on the host cell line used to express the protein as well as the culture conditions. Such modifications may include variations in glycosylation, methionine oxidation, diketopiperizine formation, aspartate isomerization and asparagine deamidation. A frequent modification is the loss of a carboxy-terminal basic residue (such as lysine or arginine) due to the action of carboxypeptidases (as described in Harris, R. J. Journal of Chromatography 705:129-134, 1995).

One aspect of the present disclosure relates to a plurality of recombinant antibodies generated by any of the methods provided herein. The present disclosure also provides a plurality of isolated polynucleotides, wherein each of the isolated polynucleotides encodes one of the plurality of recombinant antibodies disclosed herein. The present disclosure also provides a plurality of polynucleotide constructs, comprising the isolated polynucleotides. Also provided is a plurality of host cells comprising the plurality of isolated polynucleotides or the plurality of polynucleotide constructs disclosed herein. The host cells can be CHO cells.

### 7.7. Methods of Treating a Disease Responsive to a Recombinant Polyclonal Antibody

In one aspect, methods are presented for treating a subject having a disease responsive to the recombinant polyclonal antibodies. In another aspect, methods are presented for preventing a disease responsive to the recombinant polyclonal antibodies.

The disease can be a viral infection, e.g., HBV. In some embodiments, the method comprises administering the pharmaceutical composition to a patient infected with HBV.

In vivo and/or in vitro assays may optionally be employed to help identify optimal dosage ranges. The precise dose to be employed in the formulation will also depend on the route of administration, and the seriousness of the condition, and should be decided according to the judgment of the practitioner and each subject's circumstances. Effective doses may be extrapolated from dose-response curves derived from in vitro or animal model test systems.

In some embodiments, the patient had exposure to HBV by parenteral exposure, direct mucous membrane contact, or oral ingestion involving HBV surface antigen-positive materials such as blood, plasma, or serum. In some embodiments, the patient is an infant born to a HBV surface antigen-positive mother. In some embodiments, the patient has HBV exposure by sexual exposure to a HBV surface antigen-positive person or household exposure to a person with acute HBV infection. In some embodiments, the patient has chronic HBV infection.

The actual amount administered, and rate and time-course of administration, will depend on the nature and severity of disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

In some embodiments, the pharmaceutical composition is administered by injection, infusion, or by topical application. In some embodiments, the pharmaceutical composition is administered by intravenous infusion. In some embodiments, the pharmaceutical composition is administered by intramuscular injection, subcutaneous injection, intravenous injection, or intradermal injection

In some embodiments, the pharmaceutical composition is administered at a dose of 0.01, 0.05, 0.15, 0.20, 0.25, 0.30, 0.35, 0.40, 0.45, 0.50, 0.55, 0.60, 0.65, 0.70, 0.75, 0.80, 0.85, 0.90, 0.95, or 0.1 g active ingredient (recombinant polyclonal antibodies)/kg body weight. In some embodiments, the pharmaceutical composition is administered at a dose of about 0.01, 0.05, 0.15, 0.20, 0.25, 0.30, 0.35, 0.40, 0.45, 0.50, 0.55, 0.60, 0.65, 0.70, 0.75, 0.80, 0.85, 0.90, 0.95, or 0.1 g active ingredient (recombinant polyclonal antibodies)/kg body weight. In some embodiments, the pharmaceutical composition is administered at a dose of more than 0.01, 0.05, 0.15, 0.20, 0.25, 0.30, 0.35, 0.40, 0.45, 0.50, 0.55, 0.60, 0.65, 0.70, 0.75, 0.80, 0.85, 0.90, 0.95, or 0.1 g active ingredient (recombinant polyclonal antibodies)/kg body weight.

In some embodiments, the pharmaceutical composition is administered once a day, 2-4 times a day, 2-4 times a week, once a week, or once every two weeks.

In some embodiments, the pharmaceutical composition is administered once, twice, three times, four times, five times, or more. In some embodiments, the pharmaceutical composition is administered once a day for one, two, three, four, five, or more days. In some embodiments, the pharmaceutical composition is administered until the desired outcome is observed.

In some embodiments, the pharmaceutical composition is administered less than 1 day, 2 days, 3 days, 4 days, 5 days, 6 days or 7 days after the exposure to HBV. In some embodiments, the pharmaceutical composition is administered less than 2 weeks, 3 weeks, 4 weeks, 5 weeks, 6 weeks, 7 weeks, or 8 weeks after the exposure to HBV. In some embodiments, the pharmaceutical composition is administered when the patient has chronic hepatitis B infection. In some embodiments, the pharmaceutical composition is administered 2 months, 3 months, 4 months, 5 months or more after hepatitis B infection.

In some embodiments, the pharmaceutical composition is administered with plasma IVIg. In some embodiments, the pharmaceutical composition is administered with a vaccine against HBV. In some embodiments, the pharmaceutical composition is administered with a HBV surface antigen.

In some embodiments, the method further comprises administering one or more additional therapeutic agents. In some embodiments, the additional therapeutic agent is an immune stimulatory or suppressive agent.

In some embodiments, the recombinant polyclonal antibody is administered in an amount sufficient as prophylaxis against infectious disease when administered to a subject. In some embodiments, the recombinant polyclonal antibody is administered in an amount sufficient to clear infectious disease in an individual actively fighting infection. In some embodiments, the pharmaceutical composition is administered at least twice, at least three times, at least four times, or more.

### 8. EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B(1992). Further, various microfluidics and molecular genomics strategies that can be used for various embodiments of the present disclosure are disclosed in PCT/US2020/030878 filed on Apr. 30, 2020; PCT/US2021/037232 filed on Jun. 14, 2021; and PCT/US2021/044523 filed on Aug. 4, 2021, which are incorporated by reference in their entireties.

### 8.1. Example 1: Generation of ABPs with Activity Against HBV from Human Donors

A library comprising more than 1,000 unique antibody sequences (rHBIG) was generated to clear and neutralize the virus and viral particles in patients with hepatitis B virus (HBV) infection. rHBIG is a fully human polyclonal antibody (pAb) (IgG1/IgK) that binds to multiple hepatitis B surface antigen (HBsAg) variants. The antibody sequences are derived from HBV vaccinated donors' peripheral blood mononuclear cells (PBMCs). The rHBIG manufacturing process, regardless of the scale used for production, demonstrated controlled and consistent manufacturing of antibodies that bind to HBsAg, neutralize virus in vitro, and prevent infection in vivo.

rHBIG (e.g., RHBIG-1) includes antibodies that target HBsAg expressed on infectious viruses (Dane particle) and non-infectious subviral particles. In chronic hepatitis B, circulating HBsAg leads to HBV-specific immune tolerance and dysfunctional anti-HBV responses preventing clearance of HBV-infected cells. Individuals with chronic hepatitis B, even those on nucleot(s)ide analogue (NA) drugs who have complete viral DNA suppression, still produce HBsAg from integrated HBV-DNA. Although treated individuals have reduced progression to severe liver disease and hepatocellular carcinoma (HCC), treatment with NAs alone does not prevent disease progression. Lower levels of HBsAg have been associated with lower risks of HCC and liver decompensation in hepatitis B envelope antigen (HBeAg)-negative patients with low HBV levels. rHBIG binds and clears viral antigens, which can be used to induce the competent anti-viral immune response capable of maintaining HBsAg suppression.

### 8.1.1. Donor Selection

Applicant previously developed a method for generating recombinant human polyclonal Ab. The methods are disclosed in Adler et al “Rare, high-affinity anti-pathogen antibodies from human repertoires, discovered using microfluidics and molecular genomics”, MAbs, 1282-1296 (2017); Keating et al “Capturing and Recreating Diverse Antibody Repertoires as Multivalent Recombinant Polyclonal Antibody Drugs” bioRxiv (2021), which are incorporated by reference in their entireties herein.

The methods were used herein for generation of polyclonal antibodies (rHBIG) for treating Hepatitis B. In particular, rHBIG was generated from various donors and blood samples (high titer donor/low titer donor; different cell types; IgL or IgK light chain) to find the optimal samples for generation of therapeutics of improved therapeutic efficacy. High titer donors have an anti-HBV antigen serum titer of 50 IU/mL or higher and low titer donors have an anti-HBV antigen serum titer lower than 50 IU/mL.

Specifically, 8 human donors (4 high titer donors and 4 low titer donors) recently vaccinated with an HBV vaccine were recruited. The donors are healthy individuals who have received the full course of three Engerix-B HBV vaccines (GlaxoSmithKline Biologicals), a vaccine indicated for immunization against infection of all known subtypes of HBV. 60 mL of blood samples were collected on day 11 (average) after the HBV vaccine boost and CD27+ cells and CD43+ cells were separately isolated from their blood samples. rHBIG generated from CD27+ cells or CD43+ cells, each generating either antibodies with IgK or IgL light chains, were tested for the high titer and low titer doors.

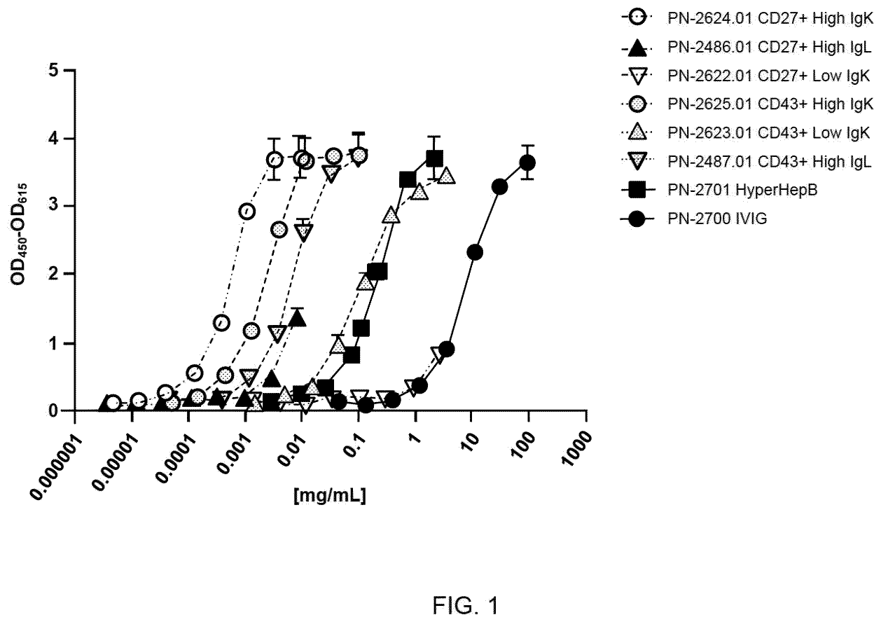

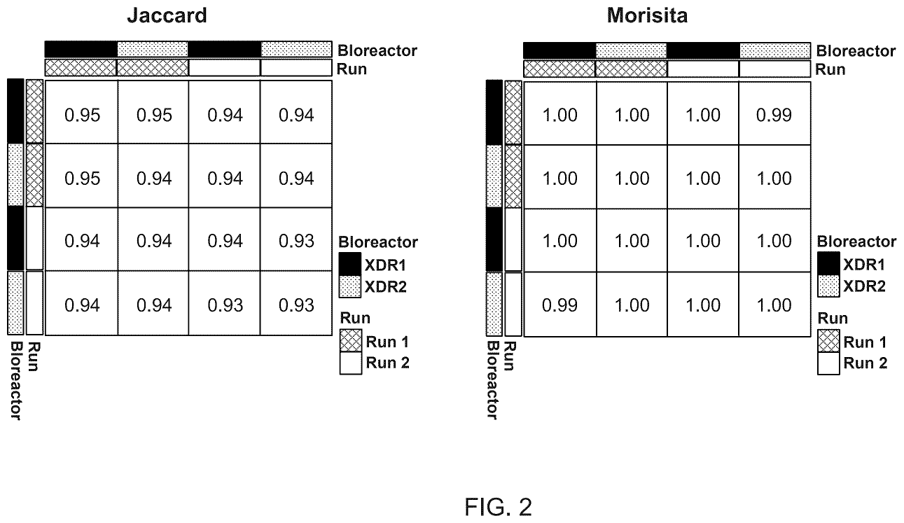

The potency of rHBIG generated from the various samples were characterized in comparison with the anti-HBV plasma hyperimmune product HyperHEP B (Grifols) and IVIG. The results are provided in FIG. 1 and Table 5 below.

TABLE 5
Potency of rHBIG compared to HyperHEP B and IVIG

| pAb library | EC50 (mg/mL) | Fold over HyperHEP B |
|---|---|---|
| IVIG | 8.27 | 0.03x |
| HyperHEP B | 0.23 | n/a |
| CD27+ High IgK | 0.00057 | 404x |
| CD27+ High IgL | 0.014* | 16x* |
| CD27+ Low IgK | 9.35* | 0.02x* |
| CD43+ High IgK | 0.0024 | 96x |
| CD43+ High IgL | 0.0069 | 33x |
| CD43+ Low IgK | 0.11 | 2x |

The results show that CD27+ cells, with IgK light chain, from high titer donors, were 404× more potent than HyperHEP B by ELISA (without any yeast display sorting), stronger than all other libraries.

### 8.1.2. Host Cell Line Development

rHBIG is a recombinant human pAb made up of antibodies derived from B cells that are naturally present in human repertoires from healthy individuals who have received the full course of three Engerix-B HBV vaccines (GlaxoSmithKline Biologicals), a vaccine indicated for immunization against infection of all known subtypes of HBV.

The RHBIG-1 antibodies were isolated from B cells derived from 17 donors exhibiting high plasma titer for binding HBsAg 8-15 days following their last Engerix-B immunization. Briefly, microfluidic methods were used to capture millions of single B cells from each donor and amplify the ribonucleic acid (RNA) of the cognate paired antibody heavy and light chains from the single cells (using the methods disclosed in Adler 2017; Keating 2021, which are incorporated by reference in their entireties herein). To maximize potency, HBsAg-specific antibody sequences were expressed on the surface of yeast as single chain variable fragment (scFv) libraries and enriched for HBsAg-specific antibodies using yeast surface display. These enriched libraries were used for the development of the recombinant pAb, RHBIG-1.

HBsAg is the target of the Engerix-B vaccine. The conserved major hydrophilic region (MHR) of HBsAg is encoded by the HBV small (S) gene. The antigens expressed on the surface of infectious virus and non-infectious sub-viral particles can be subdivided into 4 main antigen serotypes consisting of the “a” determinant with either the d/y expression at site 122 and either the w/r expression at site 160: adr, adw, ayr, and ayw. These major antigenic determinants are distributed across all nine globally circulating viral genotypes [A, B, C, D, E, F, G, H, I]. It should be noted that a single polymorphism at the amino acid 122 or 160 locations in the major antigenic region denotes a serotype change; however, the rest of the S-antigen is highly conserved. Antibodies raised against the S-antigen are broadly cross-reactive against globally circulating genotypes despite these minor amino acid differences in serotype antigen. Therefore, HBsAg-directed vaccines are used worldwide to elicit protective immune responses against globally circulating strains. There are mutations in the MHR “a” determinant that are associated with immune escape and are of clinical importance, for example M133T, I126S, and G145R. These viral mutations are problematic for mAbs targeting a single epitope, but RHBIG-1 has demonstrated binding against clinically derived HBsAg variants where mAbs fail.

Sequences of antibodies enriched for binding to HBsAg serotypes adr and adw (Prospec Bio) were engineered into full-length IgG1/IgK antibody expression constructs. This method captures substantial natural diversity from human repertoires required for binding and neutralizing hepatitis B viral variants, similar to a native immune response to a pathogen but with the added advantages of recombinant production methodology, including improved potency and manufacturing consistency (Keating 2021; Mizrahi 2022).

8.1.3. Bioproduction of rHBIG

RHBIG-1 is an anti-HBV fully human recombinant IgG1/IgK pAb produced in a Chinese hamster ovary (CHO) cell line with an engineered landing pad for site-directed integration of expression constructs. The RHBIG-1 process was developed at 10L scale and further optimized and scaled. The cell culture process is controlled from a passaging/cell age perspective regardless of production scale (cells are thawed and expanded for 4 passages prior to inoculation of the production bioreactor regardless of scale). The downstream process utilized a series of standard purification steps and viral reduction steps.

RHBIG-1 comprises >1,000 unique antibody sequences with a distribution of abundances. We use a novel process to generate a polyclonal CHO cell bank that is different from the standard process used to generate a monoclonal antibody CHO MCB (Keating 2021; Mizrahi et al 2022). The CHO cell line engineering begins with a targeted integration CHO cell line engineered from CHOZN-GS to contain a single genomic integration site with a Flp Recombinase Target (FRT) landing pad. A custom molecular cloning process was used to isolate and enrich for natively paired, full-length antibody sequences from donors vaccinated against HBV. Antibody CHO libraries were generated by co-transfecting the plasmid libraries and a plasmid expressing Flp recombinase into the targeted integration CHO cell line. For high yield clinical and commercial antibody manufacturing, multiple copies of the antibody sequence are typically integrated into the CHO genome. This would be problematic for a recombinant polyclonal product since integration of multiple plasmid copies would lead to heavy/light chain mispairing. Use of our integration cell line results in only one antibody sequence per cell. To improve the yield of a single antibody copy per CHO cell, the 2G UNic™ translational enhancer (ProteoNic Biosciences) was incorporated to increase expression while retaining the native heavy/light chain pairing.

Production is conducted under fed-batch mode and the contents of the production bioreactor undergo harvest and clarification where the harvested cell culture fluid is collected for purification. Cells are collected at the end of production for all scales of RHBIG-1 produced and monitored by Illumina sequencing of antibody RNA (antibody RNA-seq) to assess antibody diversity at the end of bioproduction. The distribution of antibodies is compared across lots to give a qualitative and quantitative indication of antibody content consistency between batches.

8.1.4. Characterization of rHBIG

The biophysical properties of RHBIG-1 were analyzed with bioinformatic methods based on the antibody sequences of its components. The molecular weight of the RHBIG-1 component antibodies ranges from 144,000 to 150,000 Da, with an average of 146,632 Da; isoelectric points (pIs) range from 7.0 to 9.5, with an average of 8.32; and extinction coefficients range from 1.250 to 1.750 ABS at 1 mg/mL with an average of 1.454 ABS at 1 mg/mL.

As no technology exists to quantify each monoclonal antibody protein component within a recombinant pAb, we perform Illumina sequencing of RNA from cells at the time of harvest and perform an informatic analysis as a monitoring method to assess the antibody population for each batch of RHBIG-1. Jaccard and Morisita indices were calculated to compare the overlap between production runs, including antibodies in the analysis that comprise >0.01% of the population. The Jaccard index compares antibody distribution regardless of abundance, whereas the Morisita compares distribution weighted for abundance. In both cases the result is reported on a scale of 0.00 to 1.00, with a higher number indicating a higher degree of overlap.

For RHBIG-1, this analysis was performed to compare two sets of two 10 L bioreactor runs performed in-house (Run 1 and Run 2 for each XDR1 and XDR2 bioreactors). For each RHBIG-1 run, 3 aliquots of cells were harvested at the end of production. From each aliquot the RNA was isolated, amplified and subjected to Illumina RNA-Seq. The abundance of each antibody presents at >0.01% was averaged across the 3 replicate samples, and the distribution from each run was compared to the other runs (FIG. 2), indicating a high degree of sequence similarity between the four replicate runs.

Further, CEX-HPLC (cation exchange high performance liquid chromatography) method was performed using a pH gradient for characterizing the charge distribution of the antibody population of recombinant pAb products. The CEX-HPLC profile of RHBIG-1 showed multiple peaks, which is consistent with its polyclonal nature.

IgGs in RHBIG-1 have different charge states, thus could be resolved by Cation Exchange-High Performance Liquid Chromatography (CEX-HPLC), involving a cation exchange chromatography with a pH gradient from a low pH (pH 5.6) mobile phase A to a high pH (pH 10.2) mobile phase B. The polarity of the IgGs charges changes depending on the buffer pH.

The following materials and equipment were used for the CEX-HPLC.

Material and Equipment

| | VENDOR/PART |
|---|---|
| ITEM | NUMBER |
| Proteomix SCX NP1.7 4.6 × 100 mm | Sepax/401NP2-4610 |
| MAbPac SCX-10 HPLC Columns, 5 μm, | Thermofisher |
| 4.6 × 250 mm | Scientific/082673 |
| CX-1 pH Gradient Buffer A (10X), | Thermofisher |
| 500 mL | Scientific/302779 |
| CX-1 pH Gradient Buffer B (10X), | Thermofisher |
| 500 mL | Scientific/302780 |
| Agilent HPLC 1260 or equivalent | NA |

HPLC Condition

| | |
|---|---|
| Mobile Phase A | CX-1 pH Gradient Buffer A, 1X (pH 5.6) |
| Mobile Phase B | CX-1 pH Gradient Buffer B, 1X (pH 10.2) |
| Column | Proteomix SCX NP1.7 4.6 × 100 mm |
| Flow Rate | 0.80 mL/min or specified |
| Column Temperature | 30° C. |
| Gradient Range | 10-100% B or specified |
| Gradient Time | 35 min or specified |

The method conditions were obtained by testing various conditions to maximize the resolution and minimize variation of the results (e.g., number of peaks over gradient length). Specifically, various flow rate, gradient of % B and gradient runtime were tested to achieve reproducibility, including column to column reproducibility and intra-method reproducibility.

To test reproducibility of the method, the same batch of the polyclonal library was tested three times under the below condition and the results are provided in FIG. 20.

HPLC Condition

| Column | Proteomix SCX NP1.7 4.6 × 100 mm | |
|---|---|---|
| Flow Rate | 0.80 | mL/min |
| Column Temperature | 30° | C. |
| Gradient Range | 10-100% B or specified | |
| Gradient Time | 35 | min |

FIG. 20 shows that the results (e.g., the peak area and height over the retention time (min)) are significantly consistent over the three separate runs showing high intra-method reproducibility.

The CEX-HPLC method was performed using three different column lots —Proteomix SCX-NP1.7 (4.6×100 mm) columns: S/N 2A54701(LN DW054), S/N 0A60382 (LN DW166), S/N 9A60383 (LN 430794). The three columns were installed at different ports and run on the same day. The results showed high column-to-column reproducibility.

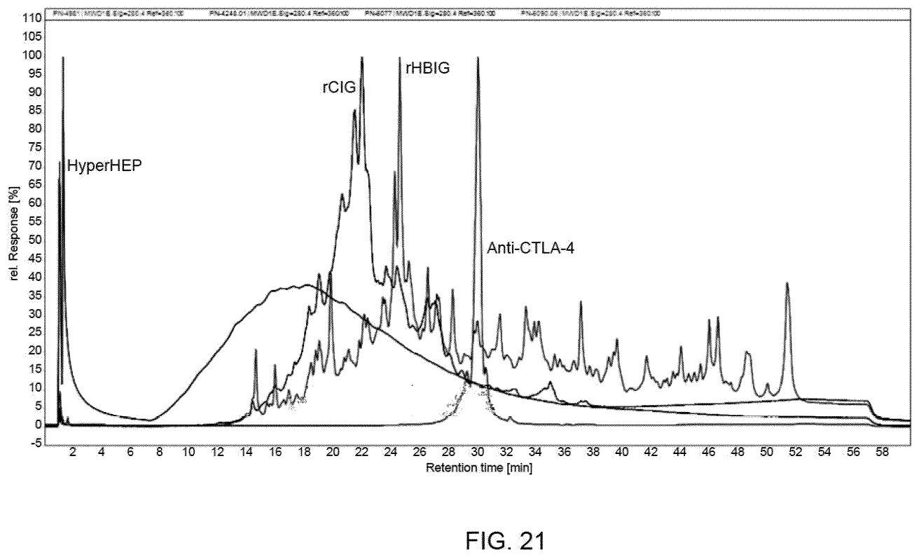

To test specificity of the CEX-HPLC method, the method was performed with different samples—anti-HBV plasma hyperimmune (HyperHEP), RHBIG-1, a library of ABPs with binding specificity to CoV antigen (rCIG), a recombinant monoclonal antibody (anti-CTLA-4). FIG. 21 provides the results. It shows that the CEX-HPLC chromatograms are specific to each sample and represent their unique characteristics. The results suggest that the method can be used to characterize and differentiate different samples.

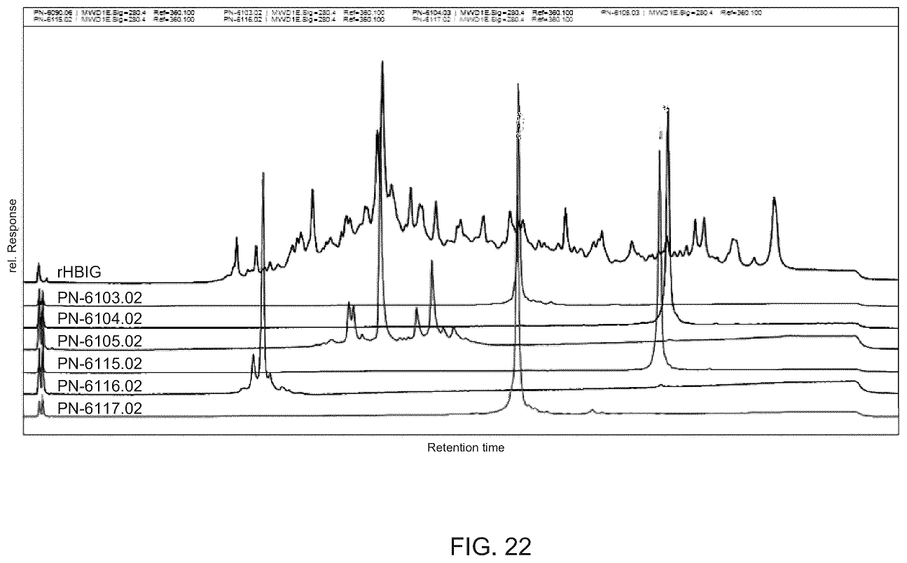

The CEX-HPLC method was performed with the library of ABPs with binding specificity to Hepatitis B virus antigen (rHBIG) and six individual antibodies in the library (PN-6103.02, 6104.02, 6105.02, 6115.02, 6116.02, 6117.02). The results are provided in FIG. 22. The results show that HPLC signals from the rHBIG represent signals from a combination of individual monoclonal antibodies in the library.

These studies suggest that the CEX-HPLC method can distinguish rHBIG from anti-HBV hyperimmune product (HyperHEP), monoclonal antibody (anti-CTLA-4), or a different recombinant polyclonal antibody (anti-CoV-2; rCIG). The analysis provides reproducible profiles for the same sample in the same method as well as for the same sample from different lots of the same column. These together suggest that the CEX-HPLC method is reliable and suitable for fingerprint type identity test for a library of ABPs.

8.1.5. Potency of rHBIG

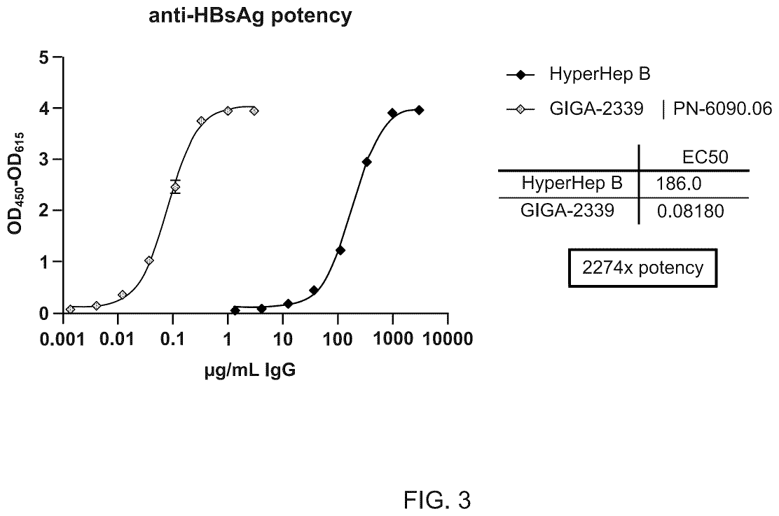

The potency of RHBIG-1 was characterized in comparison with the anti-HBV plasma hyperimmune product HyperHEP B (Grifols), using a HBsAg binding ELISA. The dose response curves are of comparable shape (FIG. 3). Based on the EC50 calculation, RHBIG-1 is greater than 2000-fold more potent than HyperHEP B.

As part of the assessment of neutralization activity, RHBIG-1 was characterized by an R&D live virus neutralization assay. This assay evaluated the neutralization capacity of RHBIG-1 against HBV virus as determined by prophylactic protection of infection in primary human hepatocytes. In brief, multiple dilutions of RHBIG-1 were mixed with 1000 multiplicity of infection (MOI) of HBV virus (defined as the number of HBV genome equivalents, as determined by quantitative PCR quantitation, added per cell in the respective growth format) for 2 hours to allow antibody binding to surface HBV antigens and formation of immune complex. After 2 hours, 200 μL of virus/antibody mixture was then added to cultured primary human hepatocytes. After 18 hours, cells were washed 5 times with fresh media then fresh media was added; this wash process was repeated at 24 hours, 4 days, and 7 days. Supernatant was collected on day 10 after exposure to antigen/antibody mixture. Total extracellular HBV-DNA copy number was evaluated by quantitatively determining extracellular HBV-DNA in treated and untreated (virus control) media via quantitative PCR. The percentage of hepatocytes that were infected was calculated for each treatment and titration point compared to a no treatment control.

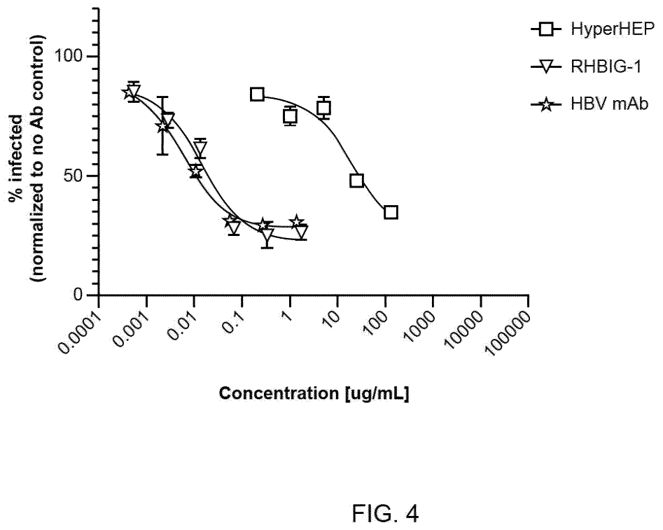

In this assay, the neutralization capacity of RHBIG-1 was compared to IVIG, HyperHEP B, and an HBV-specific monoclonal antibody. These data highlight that RHBIG-1, when exposed to live HBV virus, binds in a neutralizing manner that prevents infection of hepatocytes at ng/mL concentrations (FIG. 4). As detailed in Table 6, the IC50 (half maximal inhibitory concentration) of RHBIG-1 is far lower than HyperHEP B (>1000×) and IVIG (>50,000×, not shown) indicating a much greater neutralization capacity of HBV virus.

TABLE 6
IC50 of antibody from in vitro neutralization

| | IVIG | HyperHEP B | RHBIG-1 | HBV mAb |
|---|---|---|---|---|
| IC50 [μg/mL] | 1284 | 21.62 | 0.015 | 0.006 |

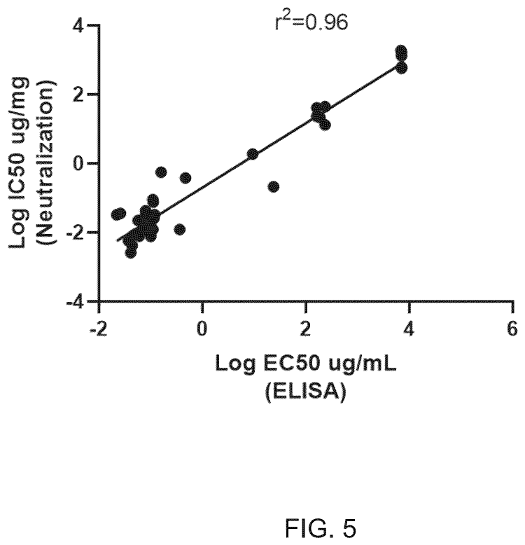

To date, several lots of RHBIG-1, as well as material made from precursor cell banks during product development, have been tested in the anti-HBs binding ELISA as well as the in vitro neutralization assay. The results of these two assays are significantly correlated (FIG. 5).

### 8.1.6. Evaluation of Binding of HBV Mutation Variants

The purpose of this in vitro study was to evaluate the capacity of RHBIG-1 to bind selected mutational variants of HBV protein antigen using an ELISA assay. The central core of HBsAg spans amino acids 99 to 169, with the antigenic domain (“a” determinant) spanning from amino acids 124 to 147; this protein loop is exposed to the surface and is recognized and bound by anti-HBs antibodies. Mutations to this “a” determinant region therefore pose a risk to generation of viral escape variants when antibodies lose the ability to recognize and neutralize a specific HBsAg epitope. In this assay, RHBIG-1 binding capacity to ten different mutations within the “a” determinant loop was measured, where each individual HBsAg protein mutant was used as a plate coat for an ELISA assay. RHBIG-1, a monoclonal anti-HBs antibody, and negative antibody control (non-HBV binding) were added in a serial dilution to bind plates coated with supernatant from HBsAg-variant producing cells.

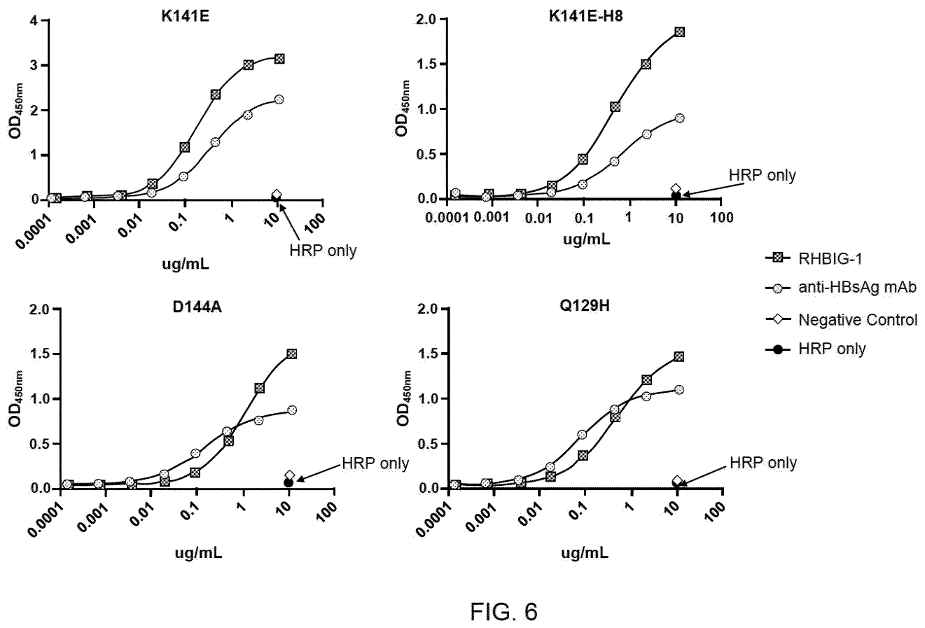

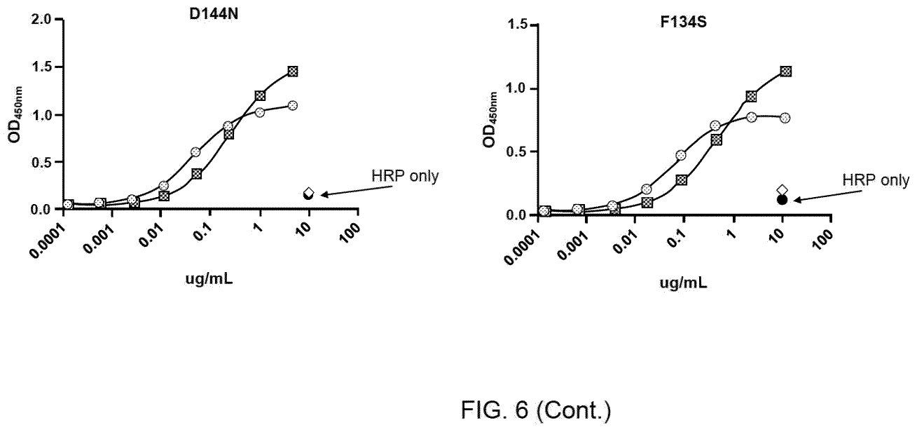

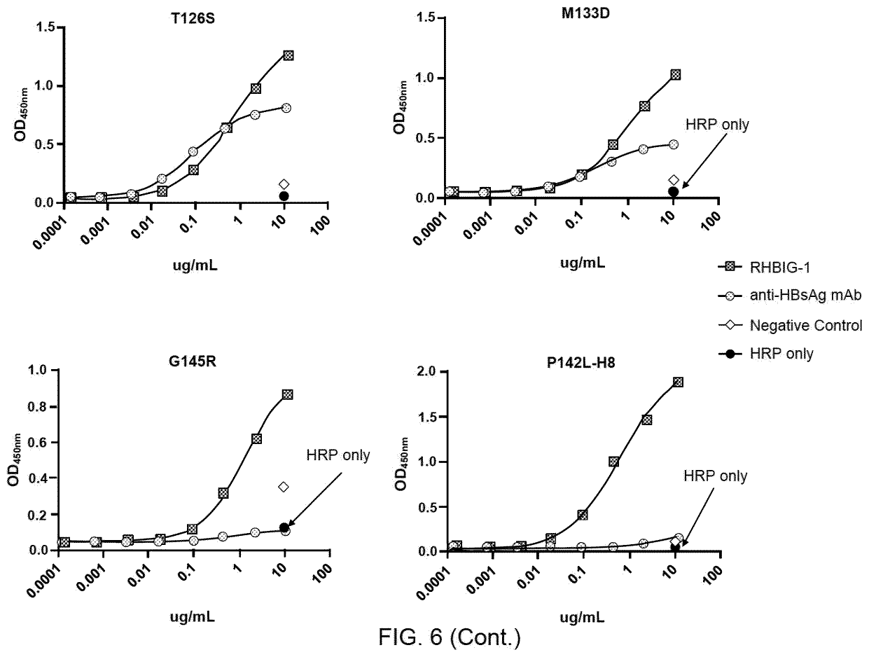

As shown in FIG. 6, RHBIG-1 binding to HBsAg (red line) was detected in all ten mutants included in this assay. Of note, RHBIG-1 was able to bind mutants G145R and P142L-H8 while the comparator monoclonal antibody (green line) was not. This result highlights the capacity of the RHBIG-1 polyclonal therapeutic to bind and therefore prevent mutational escape variants that may impact a monoclonal antibody that recognizes a single antigenic epitope.

8.1.7. In Vivo Prophylactic Protection from HBV Infection by RHBIG-1

The purpose of this in vivo study was to evaluate the prophylactic protection capacity of RHBIG-1 via neutralization of live HBV virus (genotype C) after injection in PXB mice (human hepatocyte engraftment mouse model). In brief, various concentrations of RHBIG-1 (or comparator antibody treatment HyperHEP B) were administered by IV injection to PXB mice that had been engrafted with human hepatocytes (>70%) at the study start (day −1). One day later (DO), each mouse was inoculated with HBV (1×10⁵ copies per mouse). A positive control group received no treatment prior to inoculation with HBV.

After the single dose of antibody and HBV inoculation, serum samples were taken for quantification of HBV-DNA to determine infection rates of human hepatocytes until study termination at day 70. Serum was also taken on day 57 and day 70 for analysis of HBsAg level.

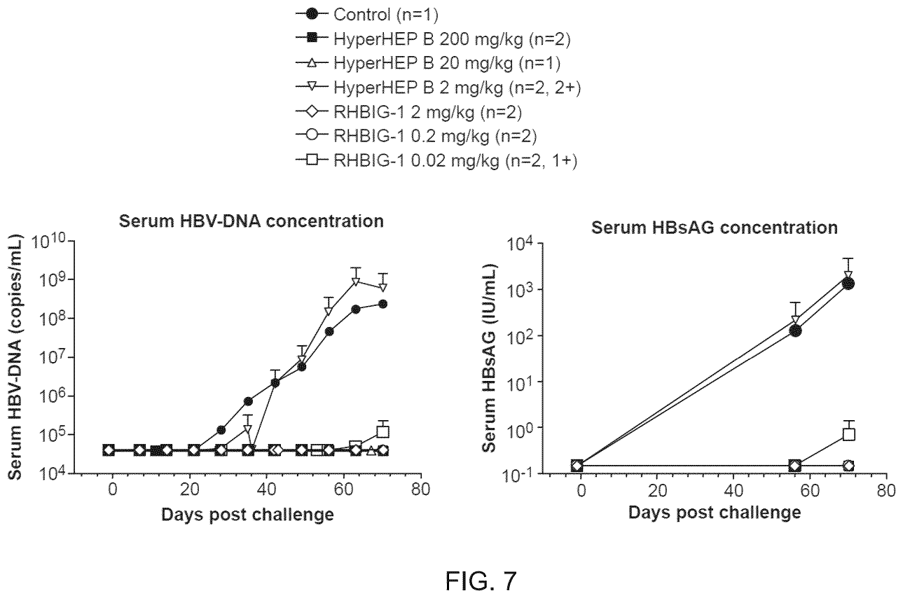

As antibody was delivered prior to HBV exposure, this study interrogated the capacity of pre-loaded antibody to bind and neutralize HBV virus before it could infect engrafted hepatocytes. Identification of HBV-DNA in the serum indicates infection of hepatocytes, with greater levels of HBV-DNA indicating increased and spreading HBV infection. Mice pretreated with 2 and 0.2 mg/kg doses of RHBIG-1 did not have detectable HBV-DNA, indicating prophylactic protection out to 70 days; these doses also did not have detectable serum HBsAg supporting the prevention of HBV infection (FIG. 7). The single dose of RHBIG-1 at 0.02 mg/kg still provided robust albeit partial prophylactic prevention of HBV infection: HBV-DNA was detected above baseline levels after 56 days although HBsAg was not detected until day 70. In comparison, the 2 mg/kg dose of HyperHEP B failed to prevent HBV infection as indicated by detection of HBV-DNA by day 28. Dose ranges of 200 and 20 mg/kg HyperHEP B prevented HBV infection, as no HBV-DNA or HBsAg was detected in these sample groups.

RHBIG-1 binds and neutralizes HBV virus present in serum and prevents infection of hepatocytes in an in vivo model. The amount of test article needed to prevent infection was much lower for RHBIG-1 compared to HyperHEP B, indicating a much greater potency.

### 8.2. Example 2: Evaluation of Binding and Neutralization of rHBIG-1

rHBIG-1 was further evaluated in a series of in vitro and in vivo nonclinical pharmacology studies to evaluate its primary and secondary mechanisms of action.

Binding of rHBIG-1 to HBsAg and live HBV virus, rHBIG-1 binding to clinically identified HbsAg variants, and neutralization of pan-genotype HbsAg were evaluated by ELISA and cell-based assays.

rHBIG-1 induction of ADCC and ADCP signal activity was measured by reporter cell-assays and FcR driven binding of rHBIG-1 to human immune cells was measured by flow cytometry.

### 8.2.1. In Vitro Pharmacodynamics

### In Vitro Binding of HBsAg by RHBIG-1 (RHBIG-1)

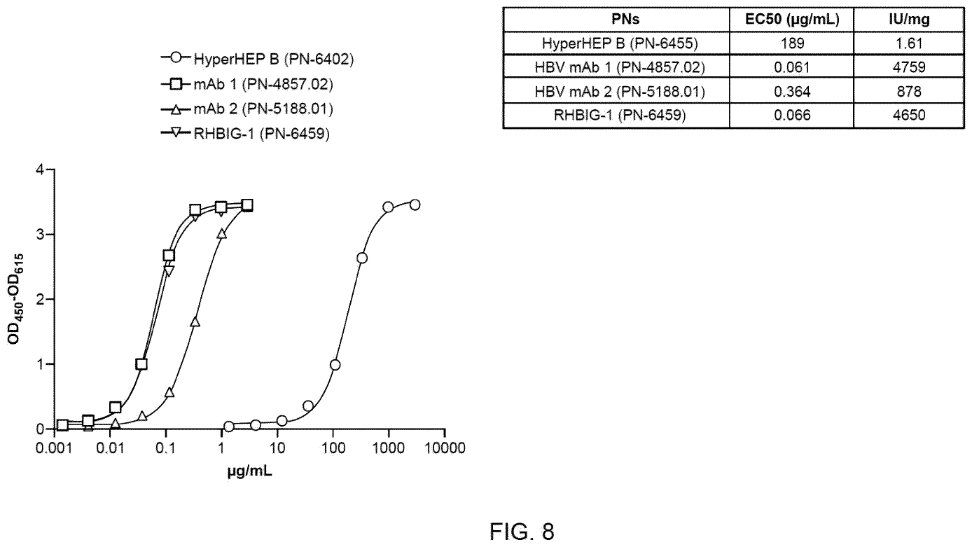

The binding capacity of RHBIG-1 against HBsAg was measured using the Monolisa anti-HBsAg ELISA (Bio-Rad, Hercules, CA), which utilizes HBsAg subtypes ad and ay as both capture and detection reagents to assess HBsAg binding. In brief, RHBIG-1, HyperHEP B (PN-6402), HBV mAb 1 (PN-4857.02), and HBV mAb 2 (PN-5188.01) were serially diluted then added to the pre-coated plates. After incubation, a horseradish peroxidase (HRP)-conjugated secondary antibody was used to detect binding; this colorimetric readout was used to calculate FC50 values for each test article. The standard curve was graphed and the titration curves for each of the test articles were interpolated to calculate the concentration of antibodies specific to HBsAg measured in IU/mg (FIG. 8). Similar to the findings of Example 1, based on the FC50 calculation, RHBIG-1 is greater than 2,000-fold more potent than HyperHEP B.

A summary of nonclinical pharmacology studies conducted to evaluate RHBIG-1 is presented in Table 7:

TABLE 7
In Vitro Pharmacodynamics

| | | | Method of |
|---|---|---|---|
| Study | Experiment | Test System | Administration |
| HBV ELISA of RHBIG-1 | Evaluation of binding potency | ELISA | In vitro |
| | of RHBIG-1 compared to | | |
| | HyperHEP B and anti-HBV | | |
| | mAb | | |
| Neutralization of HBV by | Evaluation of RHBIG-1 | ELISA | In vitro |
| RHBIG-1 | capacity to block HBV viral | | |
| | entry and infection of naive | | |
| | primary human hepatocytes | | |
| | across concentration titrations | | |
| Characterization of RHBIG- | Evaluation of RHBIG-1 binding | ELISA, WB | In vitro |
| 1 binding to HBsAg | capacity and affinity to 11 | | |
| variants and to HBV sub- | mutations in HBsAg “a” | | |
| viral particles | determinant loop region | | |
| Evaluation of FcR mediated | Induction of FcγRIIIa signaling | Cell-based Fc | In vitro |
| effector function of RHBIG- | by GIGA-2239 in complex with | receptor ADCC | |
| 1 | HBsAg | bioassay kit | |
| Evaluation of FcR mediated | Induction of FcγRIIa signaling | Cell-based Fc | In vitro |
| effector function of RHBIG- | by RHBIG-1 in complex with | receptor ADCP | |
| 1 | HBsAg | bioassay kit | |
| Characterization of human | FcR mediated binding of | Flow cytometry | In vitro |
| whole blood immune cells | RHBIG-1 antibody:antigen | | |
| that bind RHBIG-1 | complex by human blood | | |
| | immune cell subsets | | |
| Evaluation of pan-genotype | RHBIG-1 neutralization of | Quantitative | In vitro |
| neutralization capacity of | HBV genotypes using HDV-1 | immunofluorescence | |
| RHBIG-1 | pseudo type expression | | |
| Evaluation of RHBIG-1 | In vivo prophylactic protection | Male PXB mice | In vivo, IV |
| protective capacity from | from HBV infection by one IV | engrafted with | |
| live HBV infection | dose of RHBIG-1 at 2, 0.2 and | human hepatocytes | |
| | 0.02 mg/kg doses through 70 | | |
| | days post dosing in male mice | | |
| Evaluation of the | In vivo efficacy and | AAV/HBV infected | In vivo, IV |
| pharmacodynamics of | pharmacodynamics study of one | male C57BL/6J | |
| single doses of RHBIG-1 in | IV dose of RHBIG-1 at 10 | mice | |
| a chronic infection model | mg/kg and 100 mg/kg dose | | |
| | levels with immunoassay | | |
| | evaluation through 28 days after | | |
| | dosing in male mice | | |
| Evaluation of RHBIG-1 | In vivo pharmacodynamics and | AAV/HBV infected | In vivo, IV |
| impact on liver/kidney | tolerability study of one IV dose | male and female | |
| function, complement | of RHBIG-1 at 10 mg/kg and | C57BL/6J mice | |
| activation and coagulation | 100 mg/kg dose levels through | | |
| and PK/PD in an antigenic | 14 days after dosing in male and | | |
| system | female mice. | | |
| | In vivo pharmacokinetics and | | |
| | pharmacodynamics study of one | | |
| | IV dose of RHBIG-1 at 10 | | |
| | mg/kg and 100 mg/kg dose | | |
| | levels over 35 days after dosing | | |

Abbreviations:
AAV/HBV = adeno-associated virus vector for delivery of HBV;
ADCC = antibody-dependent cell-mediated cytotoxicity;
ADCP = antibody-dependent cell mediated phagocytosis;
ELISA = enzyme-linked immunosorbent assay;
Fc = fragment crystallizable region;
FcγR = Fc gamma receptor;
HBV = hepatitis B virus;
HDV = hepatitis D virus;
IV = intravenous;
mg/kg = milligram per kilogram;
PK/PD = pharmacokinetics and pharmacodynamics;
PXB = mouse model that is heterozygous for the cDNA-uPA (urokinase) transgene and possessing the SCID (severe combined immunodeficiency) trait with human hepatocyte engraftment

### 8.2.2. Primary Pharmacodynamics

RHBIG-1 was evaluated in a series of in vitro and in vivo nonclinical pharmacology studies to evaluate its primary and secondary mechanisms of action. Binding of RHBIG-1 to HBsAg and live HBV virus, RHBIG-1 binding to clinically identified HBsAg variants, and neutralization of pan-genotype HBsAg were evaluated by ELISA and cell-based assays. RHBIG-1 induction of ADCC and ADCP signal activity was measured by reporter cell-assays and FcR driven binding of RHBIG-1 to human immune cells was measured by flow cytometry.

In vivo, RHBIG-1 prophylactically prevented HBV infection in an immune-deficient murine model engrafted with human hepatocytes. RHBIG-1 engaged and neutralized HBsAg in a dose dependent manner in a chronic infection murine model, and RHBIG-1 was shown to be well tolerated in this murine chronic infection model.

### 8.2.2.1 In Vitro Pharmacodynamics

### In Vitro Binding of HBsAg by RHBIG-1

The binding capacity of RHBIG-1 against HBsAg was measured using the Monolisa anti-HBsAg ELISA (Bio-Rad, Hercules, CA), which utilizes HBsAg subtypes ad and ay as both capture and detection reagents to assess HBsAg binding. In brief, RHBIG-1 (PN-6459), HyperHEP B (PN-6402), HBV mAb 1 (PN-4857.02), and HBV mAb 2 (PN-5188.01) were serially diluted then added to the pre-coated plates. After incubation, a horseradish peroxidase (HRP)-conjugated secondary antibody was used to detect binding; this colorimetric readout was used to calculate EC50 values for each test article. The standard curve was graphed and the titration curves for each of the test articles were interpolated to calculate the concentration of antibodies specific to HBsAg measured in IU/mg (FIG. 8). Based on the EC50 calculation, RHBIG-1 is greater than 2,000-fold more potent than HyperHEP B.

In summary, this assay supports the mechanism of action of RHBIG-1 and its ability to bind to HBsAg. Furthermore, the comparison of RHBIG-1 to other antibody products provides information on relative potency. While RHBIG-1 shows a similar potency to mAb 1 and 5-fold increased potency compared to mAb 2, it shows a greater than 2,000-fold increase in potency compared to HyperHEP B.

### In Vitro Neutralization of HBV by RHBIG-1

The neutralization capacity of RHBIG-1 against HBV virus was determined using a cell-based assay to measure prophylactic protection of infection in primary human hepatocytes. In brief, RHBIG-1, HyperHEP B (PN-6402), HBV mAb 1 (PN-4857.02), HBV mAb 2 (PN-5188.01), and IVIG (Grifols; PN-6455) were mixed with 1,000 multiplicity of infection (MOI) of HBV virus (defined as the number of HBV genome equivalents, as determined by qPCR quantitation, added per cell in the respective growth format) to allow antibody binding to surface HBV antigens and formation of immune complex. The virus/antibody mixture was then added to primary human hepatocytes and supernatant was collected on day 10 after viral infection. Total HBsAg was measured using an Ig Biotechnology (Burlingame, CA) chemiluminescent assay in treated and untreated (virus control) media (HBsAg CLIA kit; Ref CL18002, readout is relative luminescence units, RLUs). From the RLUs measured for each test well, the amount of HBsAg (IU/mL) in each sample was determined, which corresponded to infected hepatocytes.

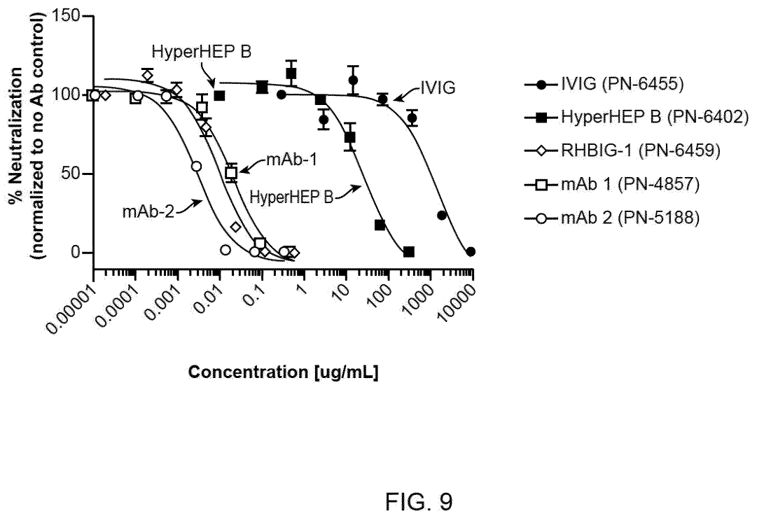

Each test sample was compared to no antibody control. The values reported below ( Table) correspond to the concentration of test article required to the dose of drug that gave half maximal inhibitory concentration values (IC50) (FIG. 9).

When exposed to live HBV virus, RHBIG-1 bound in a neutralizing manner that prevented infection of hepatocytes at 0.0097 μg/mL concentrations, similar to that of the monoclonal antibody comparators. As detailed in

Table, the IC50 (half maximal inhibitory concentration) of RHBIG-1 is far lower than HyperHEP B (>2,000×), indicating a much greater neutralization capacity of HBV virus.

TABLE 8
IC50 of antibody from in vitro neutralization assay

| | | HyperHEP | RHBIG- | HBV | HBV |
|---|---|---|---|---|---|
| | IVIG | B | 1 | mAb 1 | mAb 2 |
| | (PN- | (PN- | (PN- | (PN- | (PN- |
| | 6455) | 6402) | 6459) | 4857) | 5188) |
| IC₅₀ | 1,354 | 26.71 | 0.0097 | 0.003 | 0.021 |
| [μg/mL] | | | | | |

IC50 = Half maximal inhibitory concentration

In summary, this in vitro neutralization assay provided supporting evidence for the primary mechanism of action of RHBIG-1 to bind HBsAg on live HBV virus and neutralize the virus, such that it was unable to infect primary hepatocytes. The potency of RHBIG-1 was far greater than HyperHEP B, and it was similar, though slightly lower, in neutralization potency to two monoclonal antibodies; lower neutralization potency is likely due to the diversity of HBV epitope binding such that the potency curve is the average neutralization of all antibody species in RHBIG-1. The slightly lower potency of RHBIG-1 compared to a monoclonal antibody is not considered detrimental, as the benefits of the polyclonal mixture to bind HBV mutant variants remains highly desirable.

### In Vitro Evaluation of Binding of HBV Mutation Variants by RHBIG-1

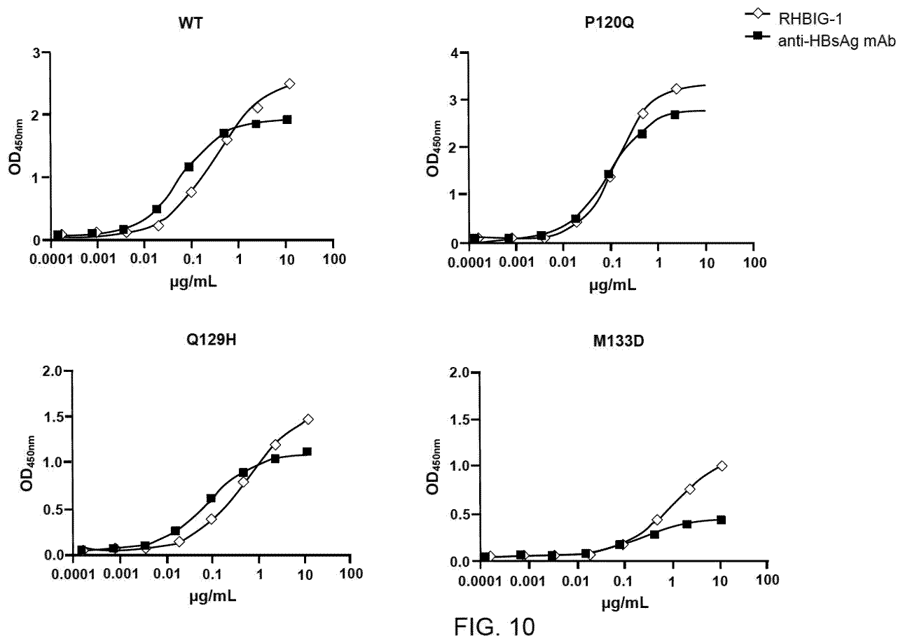

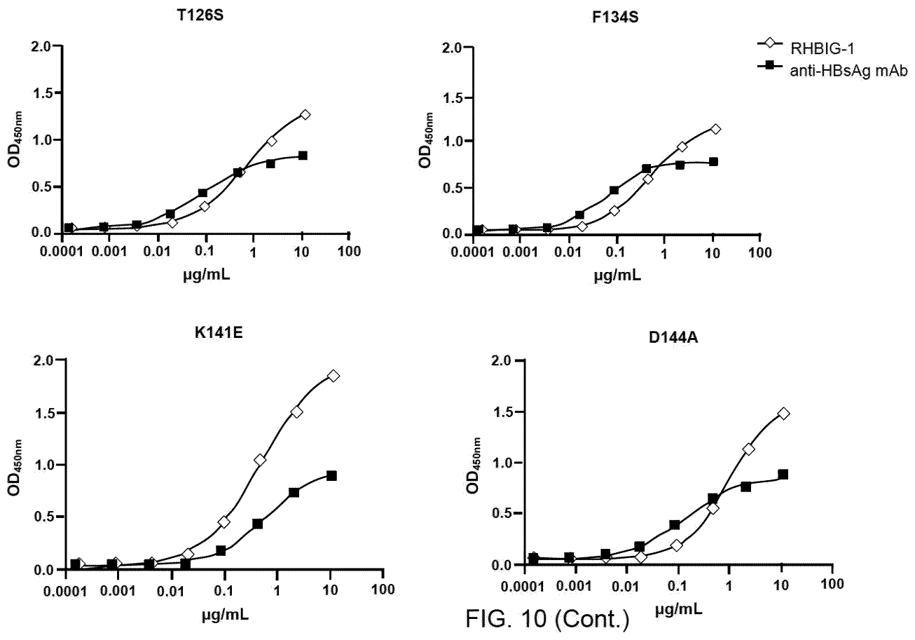

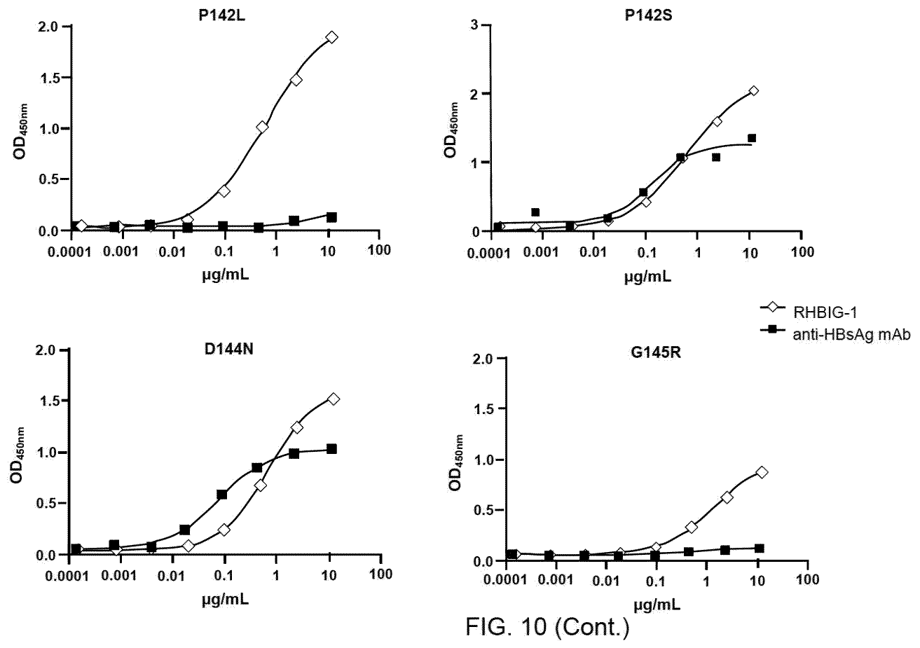

This in vitro antigen binding experiment was performed to evaluate the capacity of RHBIG-1 to bind selected mutational variants of HBV protein antigen using an ELISA assay. As stated in Example 1, the central core of HBsAg spans amino acids 99 to 169, with the antigenic domain (“a” determinant) spanning from amino acids 124 to 147; this protein loop is exposed to be recognized and bound by anti-HBs antibodies. In this assay, RHBIG-1 binding capacity to eleven different mutations within the “a” determinant loop were measured, where each individual HBsAg protein mutant was used as a capture reagent for an ELISA. RHBIG-1 and mAb 1 (PN-4857.02) were added in a serial dilution to bind plates coated with supernatant from HBsAg-variant producing cells. Secondary antibody was used to detect antibody captured in the assay and quantitation of HRP signal indicated the amount of test article bound to the mutant protein. As shown in FIG. 10, RHBIG-1 binding to HBsAg (red line) was detected in the wildtype antigen and all eleven mutants included in this assay. Of note, RHBIG-1 was able to bind mutants G145R and P142L while the comparator mAb (green line) was not.

Additional confirmation of the differentiated binding of RHBIG-1 to “a” determinant mutants, SVPs were produced expressing either wildtype HBsAg or P142L mutant HBsAg on the surface. The constituent proteins of the SVPs were analyzed by Western blot (WB) by separating via sodium dodecyl sulfate polyacrylamide gel electrophoresis, blotting onto polyvinylidene difluoride membranes, and probing with either RHBIG-1 or anti-HBV mAb 1. Anti-human IgG-HRP was used as a detection reagent and a chemiluminescent substrate was implemented to develop a signal corresponding to the amount of binding of the antibodies to HBsAg. In agreement with the ELISA binding results, by WB RHBIG-1 bound to both wildtype and P142L mutant HBsAg, while HBV mAb 1 only bound to wildtype HBsAg.

In summary, this study provided evidence that mutations to the “a” determinant region that are responsible for mutational escape from a monoclonal antibody are bound by the polyclonal antibody RHBIG-1. These results highlight the capacity of RHBIG-1 to prevent HBV from achieving mutational escape.

### In Vitro Evaluation of HBV Pan Genotype Neutralization by RHBIG-1

The purpose of this study was to evaluate the capacity of RHBIG-1 to bind and neutralize eight of the major genotypes of HBV (A-H) in vitro using HDV pseudo-expression of HBV genotype HBsAg.

HBV is divided into nine major genotypes, A through I, based on sequence divergence, which are dispersed geographically around the world. HBV genotypes vary in frequency worldwide, with genotypes B, C, and D accounting for over 60% of global infections while F, G, H, and I combined account for less than 2% total. Certain geographic regions have higher frequencies of select. With such global diversity of HBV genotype, it is clinically relevant that an anti-HBV antibody therapy be able to bind and neutralize in a pan-genotype manner.

The different HBV genotypes exhibit heterogeneity in infection and disease progression, which is evident in vitro by the varied replication rate and quantity of viral particle production. As a result, certain HBV genotypes (such as genotype G) have remained difficult to study in vitro when using live HBV to infect cells for the generation of new infectious particles. To address this issue, in vitro models have been generated that use HDV-1 to produce ribonucleoproteins combined with HBV derived envelopes in HuH7-NTCP cells (a permanent hepatocyte cell line established from hepatoma tissue expressing NTCP to allow native infection). The result is infected cells that led to the production of replication and infection competent HDV-1 virions expressing the different HBV genotype surface antigens.

Differentiated HepaRG liver progenitor cells (dHepaRG) are susceptible to infection by HDV and HBV and thus are an optimal surrogate for primary human hepatocytes (PHH). To evaluate this in vitro model system using HepaRG cells in comparison to prior neutralization data of HBV-D presented in Example 1 using PHH, live HBV genotype D (MOI of 100) was co-incubated with RHBIG-1, mAb 1 (PN-4857.02), and HyperHEP B (PN-6077) to allow antibody neutralization of HBV. The lot of RHBIG-1 used (PN-6264) in this study is comparable to the RHBIG-1 Tox Lot and GMP DS. Each antibody and HBV-D mixture was then added to dHepaRG cells and cultured for 6 days to allow infection, which was measured by quantitative immunofluorescence of HDV-1 antigen (HDAg). In agreement with Example 1, both RHBIG-1 and mAb 1 neutralized HBV-D with high efficacy compared to HyperHEP B based on the neutralization concentration 50% of antibody (NC50; the amount of antibody needed to reduce infection by 50%). RHBIG-1 (NC50 of 40.8 ng/mL) was far more effective at neutralizing HBV-D than HyperHEP B (NC50 of 44,568 ng/ml) and slightly less effective than mAb 1 (NC50 of 16.8 ng/mL). These data supported the use of dHepaRG cells instead of PHH for this neutralization assay.

Eight HuH7.5-NTCP cell lines were generated (i.e.; HuH7.5-NTCP-HBsA, HuH7.5-NTCP-HBsB, HuH7.5-NTCP-HBsC), such that HBsAg from each major HBV genotype A-H would be produced. As genotype I is rare and likely a recombinant of genotype C, it was not specifically assayed. Live HDV-1 was then used to infect each HuH7.5-NTCP-HBs cell line, resulting in production of high titers of HDV-1 that was coated in each HBV genotype of HBsAg. This method circumvented the issue of variable infectious particles production based on HBV genotype in live HBV. The live HDV-1 coated in each genotype of HBsAg was then used for infection of dHepaRG cells.

As RHBIG-1 was shown to neutralize and prevent infection of dHepaRG cells by live HBV-D, the capacity of RHBIG-1 to neutralize and prevent infection by HDV-1 coated in HBsAg from genotype D (HDV-1/D) was then evaluated. As previously described, antibody was combined with HDV-1/D and then added to dHepaRG cells for 6 days. Quantification of immunofluorescence for HDV intracellular antigen (HDAg) was used to determine infection levels of cells. In agreement with the live HBV-D neutralization, RHBIG-1 (NC50 of 10.5 ng/mL), was far more effective at neutralizing HDV-1/D than HyperHEP B (NC50 of 4,594 ng/ml) and slightly less effective than mAb 1 (3.7 ng/mL). The similar capacity to neutralize and prevent infection by HBV-D and HDV-1/D indicated that HDV-1 pseudo-neutralization was an appropriate model for measuring pan genotype neutralization by RHBIG-1.

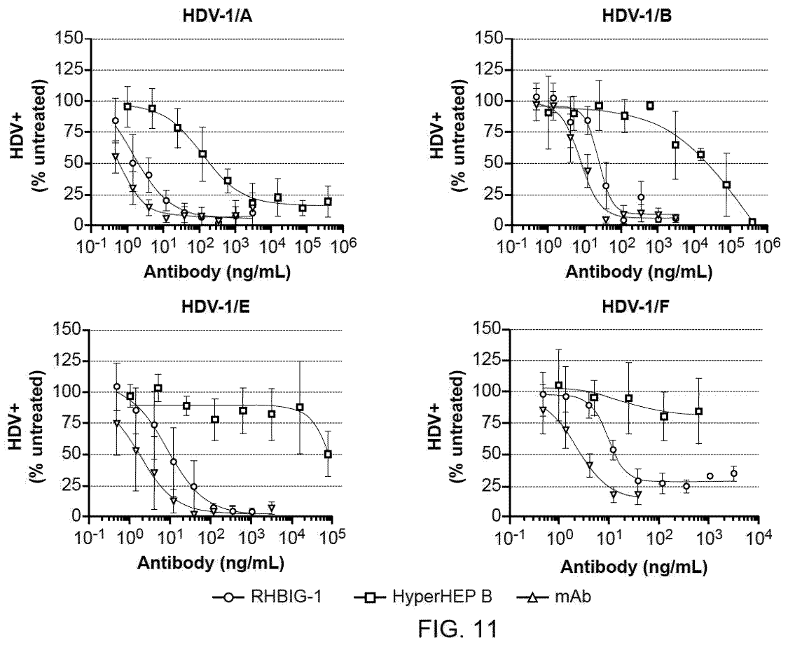

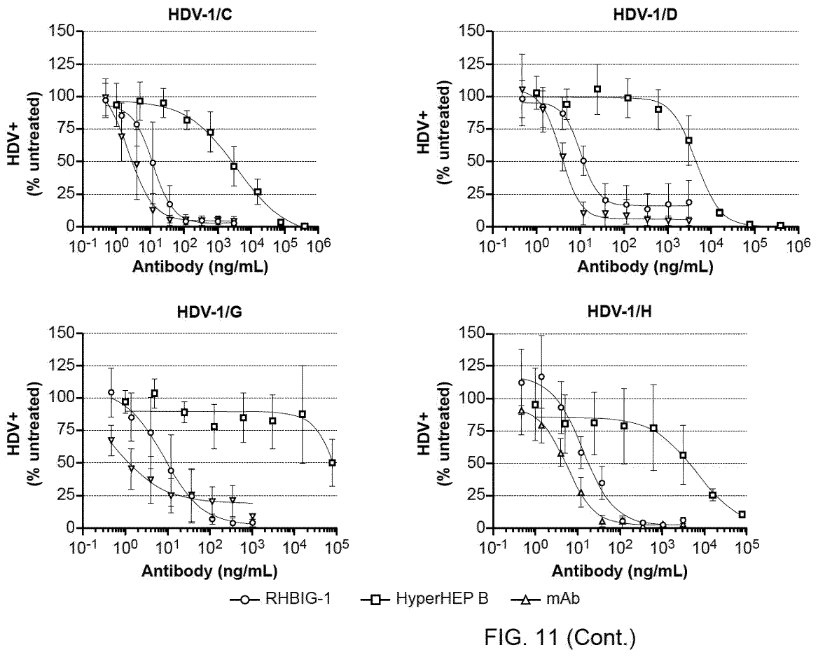

For each HDV-1 pseudotype, coated in HBsAg from HBV genotype A-H, titrations of antibody (RHBIG-1, mAb 1, or HyperHEP B) were incubated with HDV-1 before being added to dHepaRG cells and cultured for 6 days, after which immunofluorescence was used to quantify the percentage of infected cells for each condition (FIG. 11, Table 9). Cellular enumeration confirmed no toxicity of test articles across all concentrations based on viable cell count compared to puromycin treated controls. Across all eight genotypes, HyperHEP B was the least effective at preventing HDV-1/HBsAg infection of dHepaRG cells, having the highest NC50 compared to RHBIG-1 and mAb 1. In comparison to HyperHEP B, RHBIG-1 was highly effective at preventing infection of dHepaRG cells with far low ng/mL NC50 values for each pseudotype, but RHBIG-1 was consistently slightly less potent than mAb across each genotype.

TABLE 9
Neutralization concentration 50% (NC50) of HDV-1
coated with HBsAg from each HBV genotype

| | |
|---|---|
| HBV HBsAg | NC50 (ng/mL) |

| pseudotype | RHBIG-1 | HyperHEP B | mAb 1 |
|---|---|---|---|
| HDV-1/A | 1.4 | 124 | 0.55 |
| HDV-1/B | 24 | 112,175 | 7.5 |
| HDV-1/C | 12 | 3748 | 2.4 |
| HDV-1/D | 10.5 | 4594 | 3.7 |
| HDV-1/E | 8.3 | 273,494 | 1.7 |
| HDV-1/F | 11.6 | no neutralization | 3.9 |
| HDV-1/G | 4.8 | 143 | 0.6 |
| HDV-1/H | 13 | 6,563 | 5.5 |

In summary, these data indicated that RHBIG-1 can bind and neutralize the HBsAg produced from each HBV genotype. The ability to neutralize HBsAg antigen regardless of genotypic origin supports the global application of RHBIG-1, as it is efficacious against all geographic origins of HBV. Additionally, RHBIG-1 provides potent protective benefit to patients that are co-infected with multiple genotypes of HBV. As this system used live HDV for cellular infection, that RHBIG-1 potently neutralized and prevented infection of cells by HDV, indicated that RHBIG-1 could be effective in HBV/HDV coinfected patients.

### In Vitro Antibody Dependent Cellular Cytotoxicity (ADCC) Induction by RHBIG-1

The purpose of this in vitro assay was to evaluate if RHBIG-1, in complex with HBsAg, induced ADCC signal pathways. Beyond the direct impact on binding and neutralizing HBsAg, antibody therapeutics may engage secondary mechanisms of action acting through endogenous humoral immune pathways induced through FcRs. RHBIG-1, after forming an immune complex of antibody and antigen, may be recognized and engaged through its IgG1 fragment crystallizable (Fc) domain by FcγRs and result in the induction of effector functions such as ADCC for directed cell killing or ADCP of the antigen complex for processing and presentation.

In the context of ADCC, specific immune populations such as NK cells express surface FcγR for the detection of immune complex on infected cell surfaces. A few studies have described HBsAg on the membrane of infected cells, which would make ADCC potentially an important mechanism of action for an anti-HBsAg antibody therapy.

ADCC occurs primarily through the FcγRIIIa receptor, of which there are two variants. The V variant, which specifically has a higher affinity for IgG1, and the E variant, which has a lower affinity. This in vitro assay was used to evaluate the extent that RHBIG-1, which contains a human IgG1 heavy chain (the Fc region), induced ADCC signal activity when bound by the human pro-inflammatory FcgRIIIa receptor expressing the V variant.

The Promega ADCC reporter Bioassay (G7018; Madison, WI) includes effector cells (engineered Jurkat T cells) that express a high affinity (V158) variant of human surface FcgRIIIa receptor with downstream signal cascades modified to produce luciferase. FcR signal activity was quantitatively determined by luminescent output compared to controls. In this assay, RHBIG-1 was co-incubated with soluble HBsAg to allow formation of immune antibody-antigen complexes. This solution was then combined with cultured effector cells, and the engagement of receptor and downstream signal cascades was allowed to proceed for 6 hours. Luciferase activity was quantitatively determined by spectrophotometry, measured in RLUs, and was plotted against the concentration of anti-HBV-Abs. The fold of induction was calculated based on the average of the control wells with only HBsAg and effector cells. The line of best fit and EC50 were calculated on GraphPad Prism through a four-parameter variable-slope logistic curve.

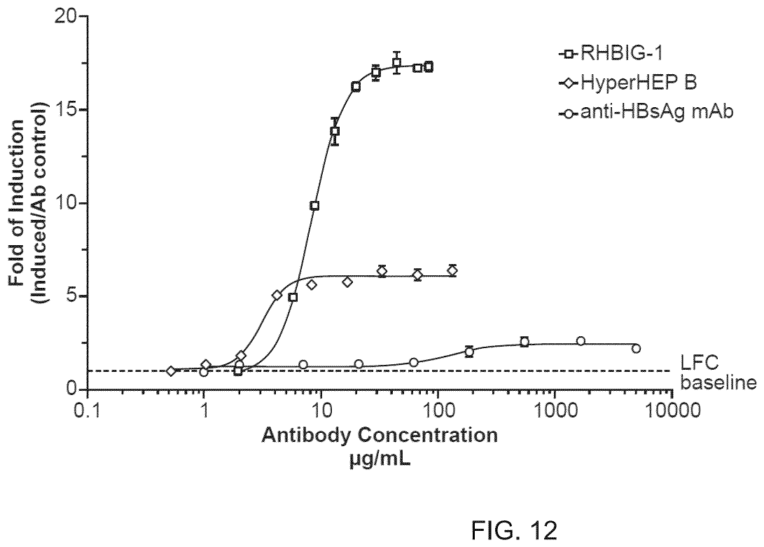

No Fc signaling activity was induced by RHBIG-1 alone (PN-6090.06), as engagement with HBsAg was necessary to induce signal activity (data not shown). FIG. 12 shows a representative ADCC assay where RHBIG-1 strongly induced an FcγRIIIa driven downstream response as determined by the log fold increase in luminescence over control levels (dotted line at Y axis=1). RHBIG-1 had a ~13-fold lower average EC50 of 8.3 μg/mL compared to 106.3 μg/mL of HyperHEP B (Table 10), indicating a much greater potency. RHBIG-1 induced a 17-log fold change (LFC) in luminescence over baseline, far greater than HyperHEP B (PN-6077) or mAb 1 (PN-4857.02) at matched concentrations of antibody, indicating greater formation of IC and higher efficiency of FcR engagement. A lower EC50 indicated a greater drug potency, and a higher maximum luminescence indicated better drug efficacy; thus, these results indicated that RHBIG-1 induced greater human FcγRIIIa signaling than HyperHEP B with this high-affinity (V158) variant of FcγRIIIa in vitro.

TABLE 10
Summary of ADCP signaling activity induction
by RHBIG-1 and Commercial HyperHEP B

| | Log Fold Change over | |
|---|---|---|
| Antibody | baseline (±SD) | EC₅₀ (±SD) μg/mL |
| RHBIG-1 (PN-6090.06) | 16.99 ± 1.21 | 8.30 ± 0.30 |
| HyperHEP B(PN-6077) | 2.30 ± 0.26 | 106.33 ± 20.69 |
| Anti-HBsAg mAb 1 | 6.19 ± 0.47 | 3.32 ± 0.18 |
| (PN-4857.02) | | |

Average results and statistics for the max RLU value as compared to its baseline and EC₅₀ as determined by luciferase quantification for RHBIG-1, HyperHEP B, and mAb 1. Data is from three separate experimental runs, shown with average and standard deviation (SD).

In summary, these data indicated that RHBIG-1 antibody, as a human IgG1 construct, was recognized and bound by human FcγRIIIa in vitro after forming an IC with HBsAg. These data suggest that RHBIG-1, when bound to HBsAg, may have the capacity to direct the secondary mechanism of action of ADCC under native conditions.

### In Vitro Antibody Dependent Cellular Phagocytosis (ADCP) Induction by RHBIG-1

The purpose of this in vitro assay was to evaluate if RHBIG-1 induced ADCP, another immune cell driven secondary mechanism of action. As mentioned in the previous section regarding ADCC, there are different types of FcRs that induce various downstream immune outcomes. While ADCC activity is mostly driven by NK cells expressing proinflammatory receptors such as FcgRIIIa, ADCP is primarily enacted through the FcgRIIa receptor (but also can include FcgRI and FcgRIIIa receptors) on phagocytic antigen presenting cells like dendritic cells, macrophages, and monocytes. There are two variants (H and R) of the FcgRIIa receptor: the H variant has a higher affinity for IgG1 than the R variant, based on the amino acid of either histidine (H) or arginine (R) at position 131. In the process of ADCP, antibody-opsonized or bound antigen is recognized by FcRs and induces uptake (phagocytosis) of target antigen for degradation. Processed antigen can also be presented on surface HLA complex for recognition by cognate T cell or B cell receptor interaction, leading to adaptive immune cell activation. In this manner ADCP links innate immunity (antigen presenting cells) and activation of adaptive immunity (T and B cells) mediated by humoral immunity.

A secondary expected benefit of reducing HBsAg titer by binding with RHBIG-1 is the concurrent easement of the systemic immunosuppressive environment induced by HBsAg; this reduction synergizes with the secondary immune activation mechanism of actions such as ADCP that antibody therapeutics can elicit. Thus, RHBIG-1 clearance of viral HBsAg may induce a host anti-viral immune response capable of maintaining HBsAg suppression, as has been demonstrated in individuals with chronic hepatitis B who control viral replication through endogenous antibody production. This in vitro assay was used to evaluate the extent that RHBIG-1, which contains a human IgG1 heavy chain Fc region, induced ADCP signal activity when bound by the human H variant FcgRIIa receptor.

The Promega ADCP reporter Bioassay (G9901) includes effector cells (modified Jurkat T cells) that express the H variant of human surface FcgRIIa receptor with downstream signal cascades modified to produce luciferase. FcR activity was quantitatively determined by luminescent signal output compared to controls. In brief, RHBIG-1 was co-incubated with soluble HBsAg to allow formation of immune complexes. This solution was then combined with cultured effector cells, and the engagement of receptor and downstream signal cascades was allowed to proceed for 6 hours. Luciferase activity was quantitatively determined by spectrophotometry, measured in RLUs, and was plotted against the concentration of anti-HBV-Abs. The fold of induction was calculated based on the average of the control wells with only HBsAg and effector cells. The line of best fit and EC50 were calculated on GraphPad Prism through a four-parameter variable-slope logistic curve.

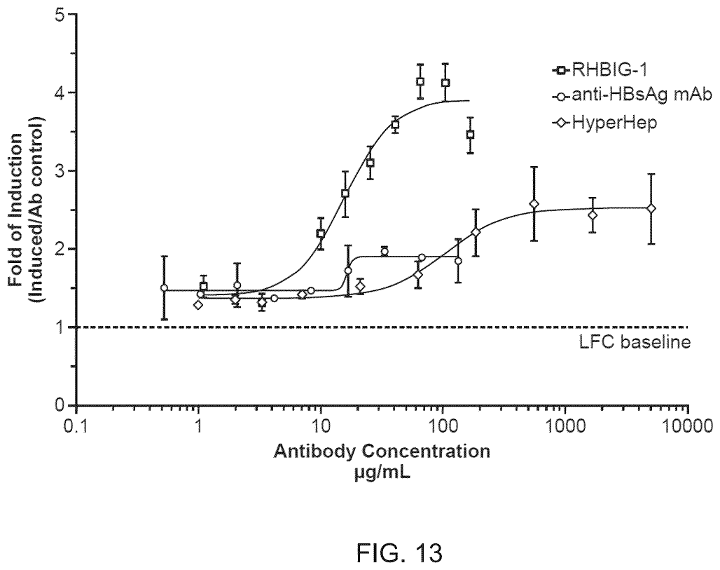

No Fc signaling activity was induced by RHBIG-1 alone (PN-6090.06), as engagement with HBsAg was necessary to induce signal activity (data not shown). FIG. 13 shows a representative ADCP assay where RHBIG-1 induced an FcgRIIa driven downstream response as determined by the log fold increase in luminescence over control levels (dotted line at Y axis=1). RHBIG-1 had a 10-fold lower average EC50 of 13.16 μg/mL compared to 131.8 μg/mL of HyperHEP B, indicating a much greater potency (Table 11). RHBIG-1 induced a 4-LFC in luminescence over background, greater than HyperHEP B or mAb at matched concentrations of antibody, indicating greater formation of IC and higher efficiency of FcR engagement.

A lower EC₅₀ indicated a greater drug potency, and a higher maximum luminescence indicated better drug efficacy; thus these results highlighted that RHBIG-1 induced more human FcγRIIa signaling than HyperHEP B or mAb 1 with the high-affinity (H131) variant of FcγRIIa in vitro.

TABLE 11
Summary of ADCP signaling activity induction
by RHBIG-1 and Commercial HyperHEP B

| | Log Fold Change over | |
|---|---|---|
| Antibody | baseline (±SD) | EC₅₀ (±SD) μg/mL |
| RHBIG-1 | 3.91 ± 0.34 | 13.16 ± 2.71 |
| (PN-6090.06) | | |
| HyperHEP B | 2.15 ± 0.33 | 131.80 ± 45.52 |
| (PN-6077) | | |
| Anti-HBsAg mAb 1 | 1.92 ± 0.30 | 12.76 ± 5.14 |
| (PN-4857.02) | | |

Average results and statistics for the max RLU value as compared to its baseline and EC₅₀ as determined by luciferase quantification for RHBIG-1, HyperHEP B, and mAb 1. Data is from three separate experimental runs, shown as average and standard deviation (SD).

In summary, these data indicated that RHBIG-1 antibody, as a human IgG1 construct, is recognized and bound by human FcgRIIa after forming an IC with HBsAg. RHBIG-1, when bound to HBsAg, may direct the secondary mechanism of action of ADCP under native conditions and thus may potentiate endogenous immune stimulation as a secondary mechanism of action.

These results show that RHBIG-1 in complex with HBsAg was necessary and sufficient to drive both ADCC and ADCP signaling activity in vitro through human FcγRIIIa and FcγRIIa, respectively. RHBIG-1 exhibited greater efficacy in induction of FcR signal activity compared to HyperHEP B and anti-HBsAg mAb 1 and was more potent than HyperHEP B in both assays. Taken together, RHBIG-1 FcγR-mediated ADCC and ADCP activity may be possible in HBV infected patients, potentially leading to antigen processing and presentation to allow for immune reactivation or directed cell killing of infected hepatocytes in vivo.

### In Vitro Evaluation of Fc Mediated Binding of RHBIG-1 in Human Blood Immune Cell Populations

The purpose of this study was to evaluate the ability of human immune cell subpopulations to interact with RHBIG-1 in complex with HBsAg via FcRs. As shown in vitro, RHBIG-1 was shown to elicit ADCC and ADCP in Jurkat effector cells expressing human FcγRIIIa or FcγRIIa, respectively.

This study was designed to assess if the human immune cells that canonically mediate ADCC and ADCP responses natively in vivo recognize and bind RHBIG-1:HBsAg IC in an in vitro assay.

In brief, RHBIG-1 was combined with soluble HBsAg to form IC, which was then incubated with whole human blood (after red blood cell lysis) to allow for FcR mediated binding to immune cell surfaces. Immune subpopulations were separated based on surface protein phenotype, as identified by flow cytometry using antibodies conjugated to fluorophores (Table).

TABLE 12
Immune cell populations phenotypic identification

| Population | Markers |
|---|---|
| Cytotoxic | CD45⁺CD19⁻CD3⁻CD11b⁺CD11c⁻HLA- |
| NK Cells | DR⁻CD16⁺CD14⁻SSC^{lo} |
| | (CX3CR1⁻CD56⁻) |
| Non-cytotoxic | CD45⁺CD19⁻CD3⁻CD11b⁺CD11c⁻HLA- |
| NK Cells | DR⁻CD16⁺CD14^{+/-}SSC^{lo} |
| | (CX3CR1⁻CD56⁺) |
| Neutrophils | CD45⁺CD19⁻CD3⁻CD11b⁺CD11c⁻HLA- |
| | DR⁻CD16⁺CD14⁻SSC^{hi} |
| | (CX3CR1⁻CD56⁻) |
| Macrophages | CD45⁺CD19⁻CD3⁻CD11b⁺CD11c⁺HLA- |
| | DR⁺CD16⁺CD14⁺SSC^{hi} |
| | (CX3CR1⁺CD56⁻) |
| Classical | CD45⁺CD19⁻CD3⁻CD11b⁺CD11c⁺HLA- |
| Monocytes | DR⁺CD16⁻CD14⁺ |
| | (CX3CR1⁻CD56⁻) |
| Intermediate | CD45⁺CD19⁻CD3⁻CD11b⁺CD11c⁺HLA- |
| Monocytes | DR⁺CD16⁺CD14⁺SSC^{lo} |
| | (CX3CR1⁻CD56⁻) |
| Non-classical | CD45⁺CD19⁻CD3⁻CD11b⁺CD11c⁺HLA- |
| Monocytes | DR⁺CD16⁺CD14⁻CX3CR1⁺ |
| | (CD56⁻) |
| Eosinophils | CD45⁺CD19⁻CD3⁻CD11b⁺CD11c⁻HLA- |
| | DR⁻CD16⁺CD14^{+/-}SSC^{hi} |
| | (CX3CR1⁻CD56⁻) |
| Classical | CD45⁺CD19⁻CD3⁻CD11b⁺CD11c⁺HLA- |
| Dendritic Cells | DR⁺CD16⁻CD14⁻ |
| (cDCs) | (CX3CR1^{+/-}CD56⁻) |
| T Cells (CD4 | CD45⁺CD19⁻CD3⁺CD11b⁻CD16⁻CD56⁻ |
| and CD8) | (CD11c⁻HLA-DR^{+/-}CD14⁻CX3CR1⁻) |
| gamma delta T | CD45⁺CD19⁻CD3⁺CD11b⁻CD16⁺CD56⁻ |
| Cells | (CD11c⁻HLA-DR^{+/-}CD14⁻CX3CR1⁻CD56⁻) |
| B Cells | CD45⁺CD19⁺ |
| | (CD3⁻CD11b⁻CD11c⁻HLA- |
| | DR⁺CD16⁻CD14⁻CX3CR1⁻CD56⁻) |

Surface protein expression (or lack of expression) criteria used to define each listed cell populations. Parentheses denote protein expression that was visually confirmed but not used as a specific gating parameter.

RHBIG-1 was biotinylated so that it could be specifically identified using a fluorophore conjugated to streptavidin. To ensure biotinylation of RHBIG-1 did not interfere with antibody functionality, the ability of RHBIG-1 to co-bind HBsAg and FcR was assessed. RHBIG-1 (PN-6090.06), biotinylated RHBIG-1 (PN-6090.06.02), and HyperHEP B (PN-6077) were assessed in the Bio-Rad HBV potency Monolisa ELISA assay. Comparable EC50 potency values between biotinylated RHBIG-1 and unmodified RHBIG-1 indicated biotinylation did not prohibit binding of HBsAg. Additionally, biotinylated RHBIG-1 was analyzed on the cell-based ADCC reporter assay and ADCP assay. Biotinylated RHBIG-1 and non-biotinylated RHBIG-1 comparably elicited ADCC and ADCP signals, suggesting that biotinylation did not block the Fc region of RHBIG-1 or its capacity to interact with FcRs. Together these data indicate that biotinylated RHBIG-1 would perform comparably to non-biotinylated RHBIG-1 in its ability to interact with FcRs in this flow cytometry assay.

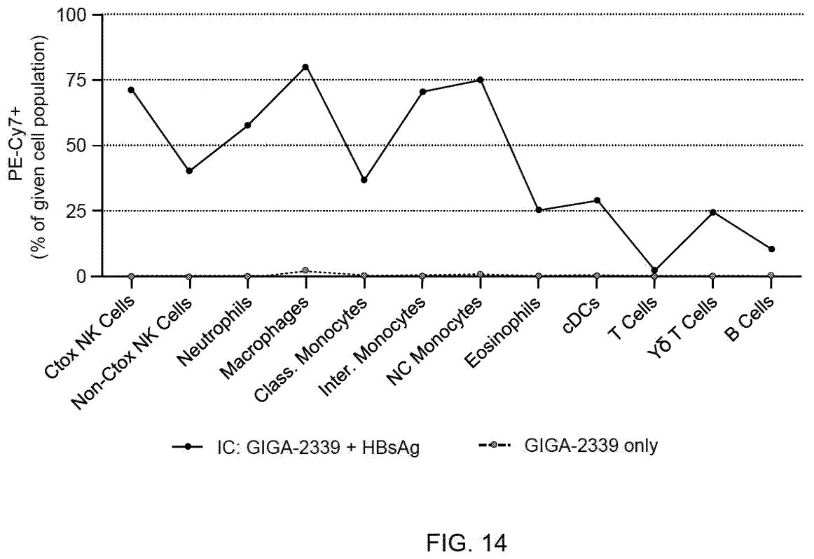

To determine if RHBIG-1 alone could bind to the surface of cells (outside of an IC), whole blood samples were incubated with RHBIG-1 only as control. Minimal RHBIG-1 signal was detected for all immune cell types (FIG. 14), indicating that leukocytes did not specifically bind RHBIG-1 alone when it was not complexed with antigen, confirming the expected need for IC formation between RHBIG-1 and antigen for FcR binding. T and B cells, which canonically have low expression of surface FcγRs, were evaluated to consider if positive IC signal could be due to direct cell binding of HBsAg by HBV specific T or B cells. Low levels of positive IC signal were detected on T and B cells (2.33% and 10.43%, respectively), which supported that specific binding to HBsAg or FcR mediated binding of RHBIG-1:HBsAg immune complex by lymphocytes was not a significant source of signal in this assay (FIG. 14). Thus, positive RHBIG-1 signal observed will only be seen when in an IC that is bound by immune cell expressed FcR.

Cells with detected surface expression of FcγRIIIa (CD16) were highly enriched in the cell population that interacted with IC, such as neutrophils and NK cells. For known FcγRIIIa+ (CD16+) immune cohorts, the percent of total population that was positive for RHBIG-1 was as follows: neutrophils—57.92%, cytotoxic NK cells—71.12%, non-classical monocytes—75.16%, intermediate monocytes—70.64%, macrophages—80.08%, and γδ T cells—24.26% (FIG. 14). Evidence of IC binding to NK cells, the primary driver of ADCC activity, supports the potential for RHBIG-1 to elicit the ADCC response in vivo. Additionally, cell types that lacked CD16 but are known biologically to express other FcγR subtypes such as FcγRIIa, that can be expressed on macrophages and classical monocytes to drive ADCP activity, were also found to be enriched for RHBIG-1 binding. IC was found associated with 80.08% of macrophages, 29.04% of classic dendritic cells, and 36.86% of classical monocytes.

None of the assay defined cell populations exhibited complete FcR binding of IC to every cell within a population, which may be due to the differential expression of FcRs within a given leukocyte population, often dependent on such factors as maturation, cellular activation, or even tissue location. Evidence of these influences with the differing percentages of IC binding cells was seen among classical, nonclassical, and intermediate monocyte subgroups.

In summary these results indicate known FcγR expressing human immune cells such as neutrophils, monocytes, and NK cells bound RHBIG-1 when in complex with HBsAg while cells with limited FcRs expression did not. There was high level of RHBIG-1 interaction with ADCC- and ADCP-driving immune cohorts when in complex with HBsAg. Taken together these data suggest that RHBIG-1, in an antigenic setting with formation of immune complex, could elicit the secondary mechanisms of action of ADCC and/or ADCP in vivo via FcγR binding on the surface of these cells.

### 8.2.2.2. In Vivo Pharmacodynamics

In Vivo Evaluation Protective Capacity from Live HBV Infection by RHBIG-1

The purpose of this in vivo study was to evaluate the neutralization capacity of live HBV by RHBIG-1 as measured by prophylactic protection capacity of human hepatocytes in male PXB mice (human hepatocyte engraftment mouse model). This study was performed by PhoenixBio (Hiroshima City, Japan). In brief, various concentrations of RHBIG-1 (PN-4863.02) or comparator antibody treatment (HyperHEP B, PN-2701) were administered by IV injection to PXB mice that had been engrafted with human hepatocytes (>70% engraftment) at the study start (day −1). One day later (day 0), each mouse was inoculated IV with HBV (1×10⁵ copies per mouse, genotype C), once test article was already present in the serum. A positive control group received no test article treatment prior to inoculation with HBV. After the single dose of antibody and HBV inoculation, serum samples were taken for quantification of HBV-DNA and HBsAg to determine infection rates of human hepatocytes until study termination at day 70.

As test article was delivered prior to HBV exposure, this study interrogated the capacity of pre-loaded antibody to bind and neutralize live HBV virus before it could infect engrafted human hepatocytes susceptible to native infection. Identification of HBsAg and HBV-DNA in the serum indicated that hepatocytes were infected, with higher levels of serum biomarkers indicating an increase and spread of HBV infection, as shown in the control mouse group (FIG. 9). Mice pretreated with 2 mg/kg and 0.2 mg/kg doses of RHBIG-1 did not have detectable HBV-DNA throughout the study, indicating live HBV was neutralized prior to successful infection of hepatocytes and prevention of HBV expansion. These RHBIG-1 doses also did not have detectable serum HBsAg, supporting the conclusion that RHBIG-1 prevented HBV infection.

### In Vivo Evaluation of Pharmacodynamics of Single Doses of RHBIG-1 in an Immune Competent Chronic AAV/HBV Infection Model

The purpose of this study was to evaluate the in vivo pharmacodynamics and neutralization efficacy of a single IV dose of RHBIG-1 (compared to PBS control, HyperHEP B, and an HBV-specific monoclonal antibody) to bind and clear HBsAg in immune intact C57BL/6J mice chronically infected with HBV (genotype D) delivered by an AAV/HBV. This study was performed by WuXi AppTec (Shanghai, China).

C57BL/6J mice were infected with AAV/HBV 28 days prior to test article dosing to ensure establishment of HBV infection and chronicity of infection. Prior to test article dosing at day 0, baseline measurements of serum HBsAg and HBV-DNA were taken at days −11 and −4 for inter-mouse tracking of changes to both biomarkers following test article dosing. Across all groups, the average baseline measurements of serum biomarkers were determined to be ~4.33 log international units (IU)/mL circulating HBsAg and ~4.25 log copy/mL HBV-DNA, confirming established HBV infection.

Test articles were IV dosed one time on day 0 of the study. This study was divided into 5 groups with 6 male mice per group for test article dosing as follows: Vehicle control (PBS), RHBIG-1 (PN-6091.17) delivered at 100 mg/kg, RHBIG-1 (PN-6091.17) delivered at 10 mg/kg, anti-HBsAg mAb 1 (PN-4857.02) delivered at 7.3 mg/kg, and HyperHEP B (PN-6077) delivered at 100 mg/kg. To evaluate the differential capacity to neutralize HBsAg given equal amounts of antibody protein, HyperHEP B and RHBIG-1 were delivered at matched protein concentrations (100 mg/kg). An anti-HBsAg targeting mAb was delivered at 7.3 mg/kg to match the IU/mg neutralization capacity of the RHBIG-1 group that was delivered at 10 mg/kg. The activity of mAb and RHBIG-1 in IU/mg was measured and determined in comparison to World Health Organization standards.

Throughout the study from test article dosing through terminal harvest 28 days later, there were no adverse clinical observations noted in the mice and body weight gain was normal indicating tolerance of the test articles.

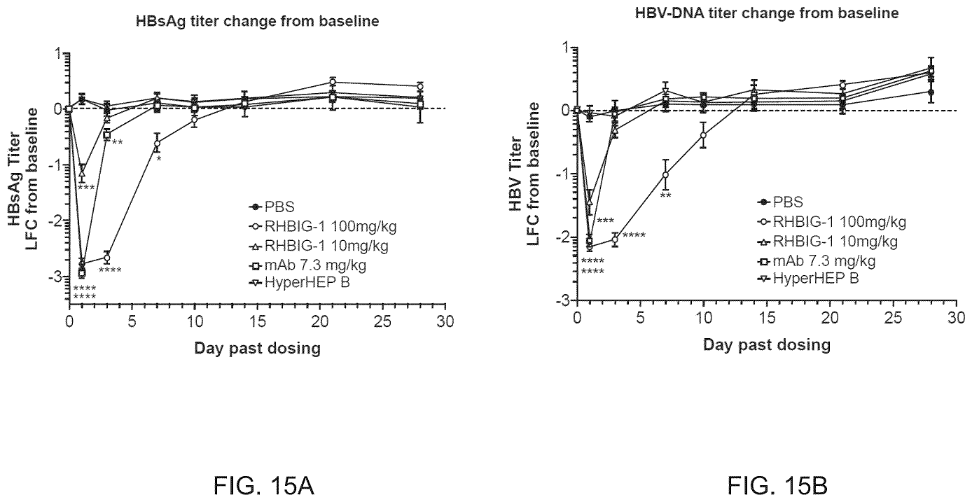

Following test article administration, measurements of plasma HBV viral load (as measured by HBV-DNA) and plasma HBsAg levels were taken at days +1, 3, 7, 10, 14, 21, and 28. Compared with the PBS control group, RHBIG-1 dose dependently reduced plasma HBV-DNA and HBsAg levels (FIG. 15). One IV dose of RHBIG-1 at 100 mg/kg significantly reduced plasma HBV-DNA levels from day 1 to day 10 and reduced plasma HBsAg levels from day 1 to day 7. One IV dose of RHBIG-1 at 10 mg/kg significantly reduced plasma HBsAg and HBV-DNA levels on day 1. Compared with the PBS control group, one dose of HyperHEP B at 100 mg/kg had no significant change plasma HBsAg and HBV-DNA levels at any timepoint. In contrast, compared with the PBS control group, one dose of mAb at 7.3 mg/kg showed a significant reduction in plasma HBV-DNA level at day 1, while plasma HBsAg level showed significant reductions at day 1 and day 3.

Peripheral blood mononuclear cells (PBMCs) were analyzed and separated into immune subsets based on surface protein staining and phenotyping using flow cytometry at day −4, and weekly after TA dosing (days+7, 14, 21, 28) to quantify any changes in circulating immune populations. Overall, there were no significant changes in PBMC subpopulation percentages in test article groups compared to PBS control.

Plasma cytokines were measured 24 hours and 28 days after test article dosing to evaluate acute and sustained changes in interferon gamma (IFNγ), tumor necrosis factor alpha (TNFα), interleukin (IL)-1b, IL-2, IL-6, and IL-12 p70 level. Cytokine measurements were below the lower level of quantitation (LLOQ) of each individual assay for all groups at both timepoints, and thus no change in cytokine level was determined.

At study completion on day 28, both the spleen and plasma were analyzed by enzyme-linked spot assay (EliSpot) to quantify IFN-γ secreting cells. Samples were stimulated with HBsAg, core peptide (as well as medium negative control and ConA positive control) before EliSpot analysis. Although there was an increase in detected spots after peptide stimulation compared to control medium in all groups, there was no significant increase in IFN-γ secreting cells from PBMCs or spleen from any test article group compared to the PBS control group. Thus, one dose of any test article did not induce additional IFN-γ secreting cell formation by day 28 above that seen in PBS treatment.

At terminal harvest, liver sections were formalin fixed and paraffin embedded (FFPE) and were stained for HBsAg using immunohistochemistry (IHC). Compared with the PBS control group, liver HBsAg positive expression in RHBIG-1 100 mg/kg, RHBIG-1 10 mg/kg, HyperHEP B 100 mg/kg, and mAb 7.3 mg/kg treatment groups was equivalent, indicating no single dose of test article induced sustained reduction of HBsAg in liver tissue.

Liver samples were also evaluated via hematoxylin and eosin (H&E) staining for histopathological evaluation. Various pathological alterations were observed in the 30 mice liver sections, including inflammatory cell infiltration in 25 out of 30 mice and necrosis in 20 out of 30 mice across PBS control and test article groups. There were individual differences of the histopathology changes within each group with clinical scoring ranging from 0 (none) to 1 (minimal) for inflammatory cell infiltration and ranging from 0 (none) to 3 (moderate) for necrosis. No tissue sections were graded 4 (marked) or 5 (severe/high) in any group. These results suggest that there was no specific induction of inflammatory cell infiltration or necrosis by test article dosing, and that these histopathological changes are a result of the AAV/HBV infection model itself.

In summary, IV delivery of RHBIG-1 resulted in the significant depletion of serum HBsAg in a dose dependent manner compared to PBS control. HBV-DNA, though not a direct target of RHBIG-1 was similarly reduced in the serum along with HBsAg. Taken together these data suggest that RHBIG-1 was able to bind to HBsAg coated viral particles containing HBV-DNA; as these particles were removed from the serum, the amount of detectable HBV-DNA in serum samples was subsequently also reduced. One single dose of RHBIG-1, mAb or HyperHEP B was not sufficient to induce detectable acute or sustained changes to serum cytokine levels, PBMC immune subset frequency, or IFN-g secreting cells in the serum or spleen. Recovery of HBsAg and HBV-DNA titers to baseline levels was seen in all test article groups by day 14 after dosing, indicating that there was no active test article present in the system to bind and deplete the constitutively produced HBsAg, which is within the expected half-life of a protein antibody delivered in the presence of serum antigen in a constitutive antigen production system. Finally, histopathological evaluation of liver 28 days after test article dosing revealed no toxicity induced by RHBIG-1.

### In Vivo Evaluation of Pharmacodynamics and Tolerability of RHBIG-1 in an Immune Competent Chronic AAV/HBV Infection Model

This study was divided into two separate arms: the purpose of arm 1 was to evaluate the systemic impact of a single IV of RHBIG-1 (at different doses) compared to PBS control in a chronic infection of male and female C57BL/6J mice by using an AAV/HBV model, with specific focus on safety, tolerability, and liver and kidney health readouts. The purpose of arm 2 of this study was to evaluate the combined pharmacokinetics and pharmacodynamics (PK/PD) of a single IV dose of RHBIG-1 (at different doses) compared to PBS control in the presence of HBsAg in male C57BL/6J mice. Both arms of this study were performed by WuXi AppTec.

### Liver and Kidney Safety Evaluation and RHBIG-1 Tolerability

As previously shown herein, RHBIG-1 neutralized HBsAg in a dose dependent manner compared to PBS control in male C57BL/6J mice infected with AAV/HBV without changes to cytokine levels or blood immune cell frequencies. Arm 1 of this study expanded upon those results by including female mice and further evaluated the systemic impact of RHBIG-1 by including measurement of CBC and coagulation measurement (by prothrombin time) at selected times after test article dosing. As it is possible that HBV antigen could be expressed on the surface of infected liver cells and therefore recognized for ADCC or complement mediated destruction, this study also included evaluation of serum liver and kidney health readouts, complement cascade activation readouts, and histology and IHC analysis of liver and kidney.

Male and female C57BL/6J mice were infected with AAV/HBV 28 days prior to test article dosing to ensure establishment of HBV infection and chronicity of infection. Prior to test article dosing at day 0, baseline measurements of serum HBsAg and HBV-DNA were taken at days −11 and −4 for inter-mouse tracking of changes to both biomarkers following test article dosing. Female mice had a lower average starting bodyweight and a lower HBsAg titer than male mice, as normally seen in this model.

Once established HBV infection was confirmed, mice were dosed IV with test articles via tail vein one time on day 0 of the study. The study was divided into 4 test groups, where each group contained 20 male and 20 female mice for test article dosing as follows: Group 1 Vehicle control (PBS), Group 2 HyperHEP B (PN-6402) delivered at 100 mg/kg, Group 3 RHBIG-1 delivered at 10 mg/kg, and Group 4 RHBIG-1 delivered at 100 mg/kg. To evaluate the differential capacity to neutralize HBsAg given equal amounts of antibody protein, HyperHEP B and RHBIG-1 were delivered at matched protein concentrations (100 mg/kg).

Following test article dosing, 5 male and 5 female mice from each group were terminally sacrificed at day +1, 3, 7, and 14. As HBsAg and HBV-DNA titers returned to baseline levels by day 14, indicating washout or depletion of all active test article, this study was designed to be completed on day 14. After test article dosing, no adverse reactions occurred in any mice and steady weight gain was recorded on average for all test groups (male and female) during the study, indicating tolerability of test article. On each terminal harvest day, whole blood was taken from each animal and was then subdivided into whole blood (for CBC assaying), plasma (for HBsAg quantitation and Prothrombin time measurement), and serum (for C3a/5a complement quantitation and liver and kidney serum protein health readouts). The liver and kidneys were harvested and underwent formalin fixation for H&E pathology and IHC readouts.

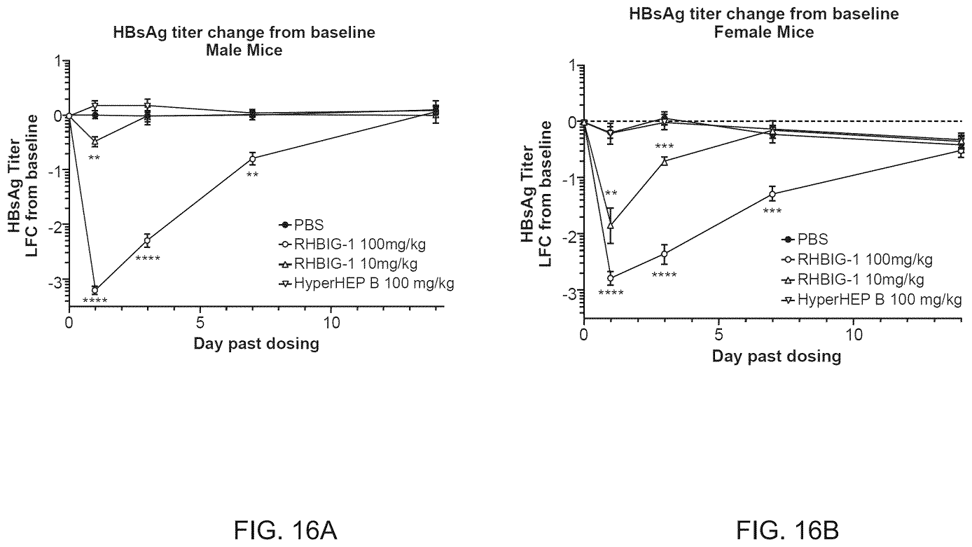

Dosing with PBS did not impact HBsAg titer, and one IV dose of HyperHEP B at 100 mg/kg did not significantly reduce HBsAg titer at any timepoint after dosing. RHBIG-1 significantly reduced HBsAg titer levels in a dose dependent manner from baseline levels in both male and female mice. RHBIG-1 had a greater log-fold reduction of HBsAg titer from baseline that persisted for a longer interval of time following dosing in female mice compared to male mice, which was likely attributable to the lower starting HBsAg titer in female mice. One IV dose of RHBIG-1 delivered at 10 mg/kg significantly reduced HBsAg below baseline at day 1 in male mice before returning to baseline by day 3; in female mice HBsAg was significantly reduced through day 3 before returning to baseline by day 7. One IV dose of RHBIG-1 delivered at 100 mg/kg significantly reduced HBsAg titer from baseline through day 7 before returning to baseline by day 14 in male and female mice, although the log fold reduction in HBsAg from baseline was greater in females at day 3 and day 7 (FIGS. 16A-16B).

At each terminal harvest timepoint, a CBC assay was performed to evaluate changes to red blood cells, white blood cells, and platelets from test article dosing. Historical C57BL/6 CBC measurements (averaged from over 100 C57BL/6 male or female mice) were used to aid identification of biologically significant changes in CBC assays. Though there were some variations outside of historical averages in CBC assay readouts, the lack of consistent change and comparison to PBS control temporally, by gender, and in a dose dependent manner for RHBIG-1, indicated biological heterogeneity rather than test article dosing impact.

Serum levels of bilirubin, creatinine, and urea were measured to evaluate test article dosing impact on kidney health. Bilirubin, a byproduct of the breakdown of red blood cells, can be elevated in the blood when liver or kidney function is compromised. Creatinine and urea are byproducts normally filtered from the blood by the kidney, thus elevated levels could indicate kidney health issues. Changes in serum concentrations for these factors were seen during the study (within normal expected ranges), but there was no consistent upward or downward trend in these changes compared to PBS control temporally, nor were changes reflected by gender, and no changes were seen in a dose dependent manner for RHBIG-1 dose groups. Taken together this indicated these changes were likely driven by natural heterogeneity rather than test article dosing impact. Thus, compared to PBS control there were no biologically relevant changes to these readouts in male or female mice across time points, suggesting no impact on kidney health due to test article dosing.

Serum levels of ALT, aspartate transaminase (AST), albumin (ALB), alkaline phosphatase (ALP), glutamate dehydrogenase (GLDH), and lactate dehydrogenase (LDH) were measured to evaluate test article dosing impact on liver health. During liver damage, liver enzymes like ALT, AST, ALP, GLDH, and LDH can leak into the bloodstream, thus elevation of these factors can indicate hepatic damage. Similarly, ALB is a key blood protein produced by the liver and loss of ALB can indicate liver dysfunction. Slight deviations in serum concentrations for these factors were seen during the study (within normal expected ranges), but there was no consistent upward or downward trend in these changes compared to PBS control temporally, nor were changes reflected by gender, and no changes were seen in a dose dependent manner for RHBIG-1 dose groups. Taken together this indicated these changes were likely driven by natural heterogeneity rather than test article dosing impact. Thus, compared to PBS control there were no biologically relevant changes to these readouts in male or female mice across all time points, suggesting no impact on liver health due to test article dosing.

Evaluation of test article dosing on coagulation was measured by prothrombin time (PT), a measurement of how long it took for a clot to form; for C57BL/6 mice the normal PT time was between 9 and 12 seconds. PT coagulation time was not impacted by gender or by test article group across all time points, with no significant change compared to the PT time of PBS control. Thus, delivery of test article and formation of antibody:antigen IC did not alter the ability of blood coagulation across time points in this study.

Interrogation of antibody driven activation of the complement cascade was evaluated by separate measurements of C3a and C5a after test article dosing; C3a and C5a are two cleavage products found in elevated levels after complement cascades have been activated. The concentration of C3a and C5a was measured compared to PBS control for each time point in the study for male and female mice. No test article groups had significantly more C3a than the PBS control mice in male or female mice at any time point, in contrast to the expectation that an antibody therapy bound to antigen would engage the complement pathway and result in elevated C3a. C5a levels exhibited slight variation compared to PBS control but lacked gender and temporal consistency and exhibited no dose dependent changes for RHBIG-1 groups. Taken together, the concentrations of C3a and C5a from the same gender, time point, and test article group do not exhibit a positive correlation as expected in the case of an engagement of the complement cascade, and this variance was not linked with other biological changes from other readouts. Thus, these results indicated that complement levels were heterogenous across timepoints but did not appear to be changing in a biologically significant manner that impacted animal health.

Standard H&E pathology examination was performed on FFPE liver and kidney sections taken from both male and female mice on terminal harvests 1, 3, 7, and 14 days after test article dosing. Various pathological alterations were observed in liver sections, including inflammatory cell infiltration in 47 out of 160 liver samples, and hepatocellular necrosis in 48 out of 160 mice; detection of these alterations was dispersed across all test groups and genders. Lesions were described in 56 liver samples with 55 scored as minimal, 1 sample was scored as mild (HyperHEP B, Male, day 1), and no samples scored as moderate or above. The liver pathology observations were distributed across male/female mice, across time points, and across all dose groups, which indicated no clear influence of test article dosing, and instead the infection model was the contributing factor. Pathology examination of the kidney tissue yielded no detectable lesions in 155 of the 160 samples across all dose groups, genders, and harvest date after dosing, supporting that test article dosing did not negatively impact the kidney tissue.

### PK/PD in an Antigenic System

Given that RHBIG-1 is an antibody therapeutic that binds an exogenous antigen for neutralization and destruction, it is possible that RHBIG-1 will be impacted by target-mediated drug disposition (TMDD) wherein its high affinity for antigen impacts the PK profile in a dose dependent manner. Initially when HBsAg levels are high, RHBIG-1 will bind available antigen and be rapidly turned over; as antigen levels are decreased through antibody mediated clearance, more RHBIG-1 will remain unbound and thus be detectable in the serum in standard assays. The result is that the PK profile is expected to be non-linear, where the half-life of RHBIG-1 is related to the concentration of serum HBsAg.

Arm 2 of this study was used to evaluate the PK/PD relationship of RHBIG-1 in a system with antigen present, where male C57BL/6J mice were infected with AAV/HBV 28 days prior to test article dosing to ensure establishment of HBV infection and chronicity of infection. Prior to test article dosing on day 0, baseline measurements of serum HBsAg were taken at days −11 and −4 for inter-mouse tracking of changes to HBsAg following test article dosing.

Once established HBV infection was confirmed, mice were dosed IV with test articles via tail vein one time on day 0 of the study. Arm 2 was divided into 2 test groups where each group contained 12 male mice for test article dosing as follows: Group 5 RHBIG-1 delivered at 10 mg/kg and Group 6 RHBIG-1 delivered at 100 mg/kg. Both groups were randomly subdivided into “A” and “B” cohorts containing 6 mice each to allow blood sampling across more time points. Mice from Group 5/6A were bled at timepoints after test article dosing as follows: 15 min, 1 hr, 8 hr, 2 days, 10 days, 21 days, and 35 days. Mice from Group 5/6B were bled at timepoints after test article dosing as follows: 30 min, 4 hr, 24 hr, 7 days, 14 days, 28 days, and at day 35 as backup samples.

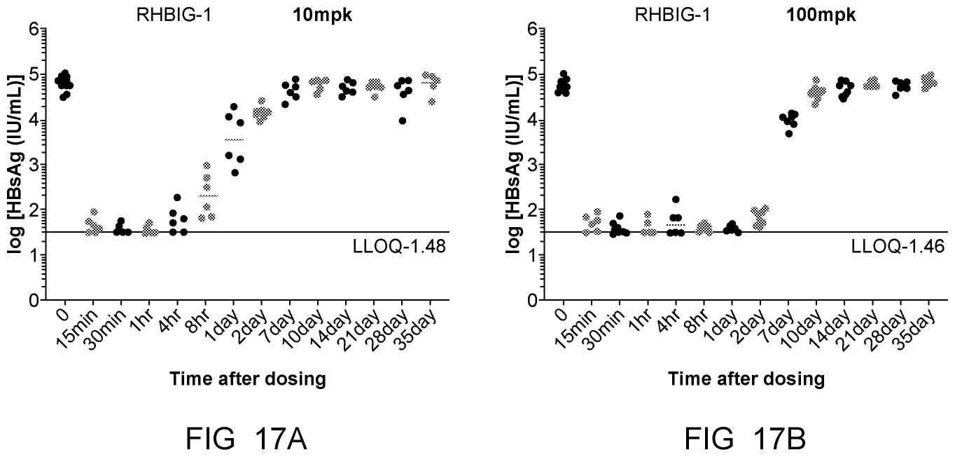

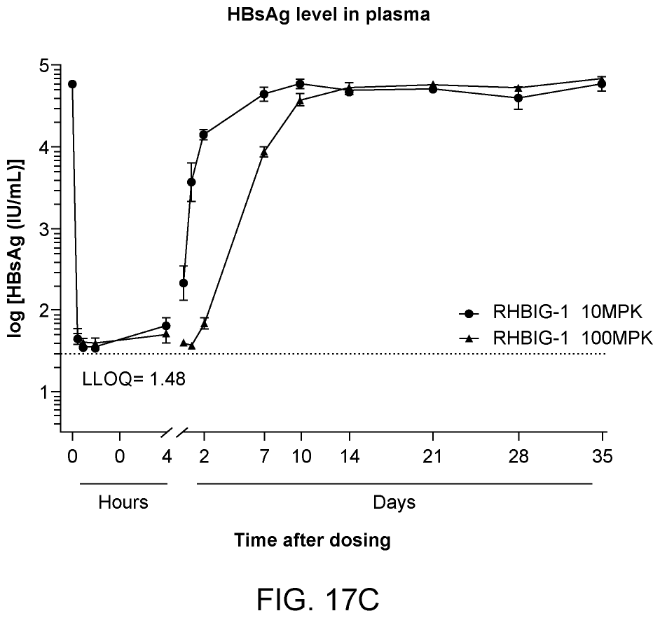

For each blood draw listed, the HBsAg titer was measured. Similar to the result seen in arm 1 of this study, RHBIG-1 exhibited a dose dependent depletion of HBsAg from the serum in this PK/PD study. The additional testing time points in the first day after dosing reveal that RHBIG-1 10 mg/kg depleted HBsAg equivalent to 100 mg/kg until 4 hours after dosing, with recovery of serum HBsAg starting by hour 8 and return to baseline seen by day 7 (FIGS. 17A-17C). The RHBIG-1 100 mg/kg dose suppressed HBsAg levels below the LLOQ through day 2 and recovery of HBsAg levels was seen at day 7 with return to baseline levels by day 14.

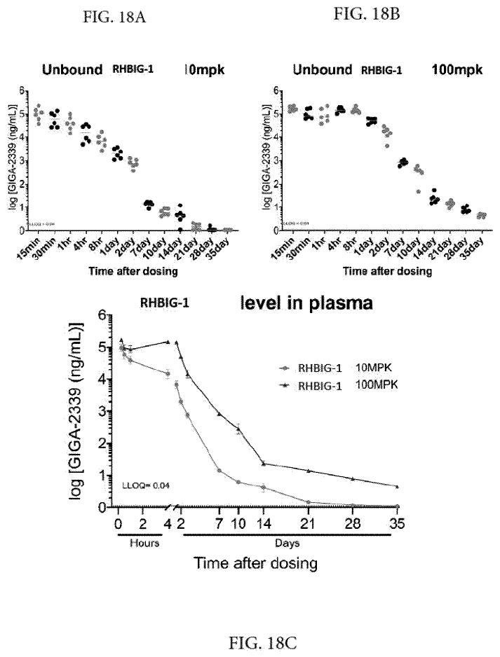

At each PD time point listed above, the amount of RHBIG-1 not bound to HBsAg in the serum was also measured. In brief, HBsAg was used as the capture antigen for an ELISA assay serving as the well plate coat. When the wells were exposed to serum, RHBIG-1 in serum would be able to bind the HBsAg capture antigen if it were not already bound to HBsAg from the serum. Complexed RHBIG-1 would be washed away and only the RHBIG-1 that was “free” (unbound to HBsAg) in the serum would be captured for detection using an anti-human anti-IgG1 antibody conjugated to HRP for colorimetric quantitation compared to standards. As expected, the quantity of detected RHBIG-1 in the 10 mg/kg dosing group declined more rapidly than the RHBIG-1 100 mg/kg group, as there was 10-fold less antibody available from study start to bind to an equivalent amount of starting HBsAg (FIGS. 18A-18C). As a result, there was no unbound RHBIG-1 detected by day 10 in the 10 mg/kg group, indicating all available antibody had been bound to HBsAg; this result aligned with the PD result showing a return to baseline of HBsAg titer by day 10 in this dose group. The RHBIG-1 cohort dosed at 100 mg/kg had detectable RHBIG-1 28 days after dosing with minimal antibody detected above the LLOQ by day 35. In this dosing cohort, the HBsAg levels were nearly at baseline beyond day 14 and remained at that level as the free RHBIG-1 present to bind and deplete HBsAg could not reduce HBsAg below replacement levels. Thus, in a constitutive HBsAg producing system, with high starting HBsAg titer, one IV dose of RHBIG-1 at 10 mg/kg depleted HBsAg below the LLOQ for 4 hours before HBsAg levels began to recover, compared to one IV dose of 100 mg/kg that depleted HBsAg below the LLOQ for up to 2 days before HBsAg levels began to recover.

In summary, RHBIG-1 bound and neutralized HBsAg in a dose dependent manner, with a greater depletion of HBsAg for a longer interval of time after dosing by the 100 mg/kg dose compared to the 10 mg/kg dose level. RHBIG-1 was present in the serum two days after dosing at 10 mg/kg but was not detected by ten days after dosing; based on the reduction of HBsAg seen at day 3 and 7 but not day 10, it is likely that all RHBIG-1 had bound to antigen (and thus no longer detectable) between day 7 and 10. When dosed at 100 mg/kg, RHBIG-1 was detected in the serum up to 35 days after dosing close to the limit of detection. Though there was remaining RHBIG-1 through this timepoint, it was not present in high enough level to reduce the constitutively produced HBsAg; the 100 mg/kg animals returned to HBsAg baseline between day 14 and day 21.

Dosing with RHBIG-1 up to 100 mg/kg did not induce significant changes to CBC, liver and kidney serum health readouts, coagulation time, or complement activation cascades, indicating safety and tolerability of RHBIG-1. Differences in serum chemistry, complement measurement, and CBC test results, statistically significant or not, between control and RHBIG-1-treated animals, were consistent with normal variation and considered incidental due to small magnitude, lack of dose relationship, inconsistency between sexes, and/or the absence of correlative findings. Pathology examination of liver and kidney tissue taken from male and female mice 1, 3, 7, and 14 days after test article dosing indicated no significant variance in levels of inflammatory immune cell infiltration and necrosis across all test article groups, including PBS control, and were thus attributable to the infection model, not as a result of test article dosing. Taken together these results support the dose dependent depletion of HBsAg by RHBIG-1 and the safety and tolerability of dosing with RHBIG-1 up to 100 mg/kg from one to fourteen days after dosing.

### In Vivo Evaluation of Pharmacodynamics of Single Doses of RHBIG-1 in an Immune Knockout Human Liver Engraftment Chronic HBV Infection Model

To ensure proper evaluation of RHBIG-1 dosing in an antigenic system, two studies in the AAV/HBV model to evaluate the efficacy and non-GLP safety/tolerability of RHBIG-1, with specific assessment of the potential for liver toxicity. Furthermore, a full GLP toxicology study was performed in Sprague Dawley rats, which did not show detrimental impact of test article dosing, during in life or recovery phases, up to 500 mg/kg. Taken together, these in vivo studies support that RHBIG-1 is safe and tolerable and exhibits dose dependent efficacy in an antigenic system.

### 8.2.3. Safety Pharmacology

Informal safety pharmacology of RHBIG-1 was conducted in immune competent C57BL/6J mice infected with AAV/HBV for constitutive production of HBsAg. No adverse changes were evident in the liver and kidney tissues (by histopathology) after administration of RHBIG-1 at dose ranges up to 100 mg/kg when evaluated at timepoints spanning one day to fourteen days after dosing. Serum evaluation of liver and kidney proteins that serve as biomarkers of organ function and health did not show RHBIG-1 dosing related changes compared to PBS control, consistent with the pathology examination result that revealed only AAV/HBV infection model driven effects. Quantitation of CBC did not reveal RHBIG-1 dosing related changes, nor were there changes to coagulation.

In a GLP study, RHBIG-1 toxicity was measured in Sprague Dawley rats after single or multiple dosing up to 500 mg/kg per week including clinical observations, body weights, food consumption, ophthalmic observations, functional observational battery, plethysmography, and clinical and anatomic pathology. No RHBIG-1 dosing related mortality or body weight changes were measured, and clinical chemistry and microscopic findings were mild and reversed in the recovery phase, indicating effects of dosing up to 500 mg/kg were considered non-adverse.

In vivo, rHBIG-1 prophylactically prevented HBV infection in an immune-deficient murine model engrafted with human hepatocytes. rHBIG-1 engaged and neutralized HBsAg in a dose dependent manner in a chronic infection murine model, and rHBIG-1 was shown to be well tolerated in this murine chronic infection model.

rHBIG-1 demonstrated an in vitro binding EC50 of 0.066 μg/mL, more than 2,000-times more potent than HyperHEP B® (Grifols, Barcelona, Spain; EC50 of 189 μg/mL), a plasma-derived immunoglobulin approved for post HBV exposure prophylaxis and for preventing hepatitis B recurrence following HBsAg-positive liver transplants. rHBIG-1 also had a calculated binding potency of 4,650 IU/mg, which when compared to HyperHEP B (1.61 IU/mg) indicates more than 2,000-times greater potency. The capacity of rHBIG-1 to bind and neutralize live HBV and prevent infection of primary human hepatocytes in vitro was measured using an enzyme-linked immunosorbent (ELISA) assay of extracellular HBV-DNA in the supernatant. The IC50 (half maximal inhibitory concentration) of rHBIG-1 to prevent infection was 0.0097 μg/mL, over 2000-times more potent than HyperHEP B (26.71 μg/mL). The polyclonal mixture of rHBIG-1 was highly potent, similar to an anti-HBsAg monoclonal antibody that exhibited an IC50 of 0.003 μg/mL.

rHBIG-1 bound 11 clinically relevant mutations within the “a” determinant loop of HBsAg as measured by an ELISA assay. By comparison, an HBsAg targeting mAb was unable to bind 2 of the mutations, highlighting that mutational escape seen with a mAb is prevented by the polyclonal rHBIG-1 mixture due to its broad diversity of HBsAg epitopes that are targeted.

The pan genotype neutralization capacity of RHBIG-1 was evaluated through a hepatitis D virus (HDV)-1 pseudo expression system, expressing HBsAg from each genotype of HBV. RHBIG-1 was able to bind and neutralize HDV-1/A, B, C, and D with potent NC50 (50% neutralization concentration) values of 2.8 ng/mL, 5.3 ng/mL, 19.9 ng/mL, and 10.4 ng/mL, respectively, far more potent than HyperHEP B which had NC50 values of 35.9 ng/mL, 1,427 ng/mL, 1,889 ng/mL, and 3,000 ng/mL respectively.

Two cell-based reporter assays measured the induction of downstream signaling activity of Fc gamma receptors (FcγRs) by RHBIG-1 in complex with HBsAg. For both ADCC and ADCP assays, RHBIG-1 induced signal activity with much greater efficiency than HyperHEP B, as determined by the log fold of induction of signal activity in both assays. For ADCC, RHBIG-1 (EC50 of 8.3 μg/mL) was ~13-times more potent than HyperHEP B (106.33 μg/mL; for ADCP signaling, RHBIG-1 (EC50 of 13.16 μg/mL) was ~10-times more potent than HyperHEP B (131.80 μg/mL).

Using an in vitro flow cytometry assay, RHBIG-1 (only when in complex with HBsAg) was co-identified with FcγR expressing human leukocytes known to induce ADCC and ADCP at high levels (co-detected with 79% of cytotoxic NK cells, 61% of nonclassical monocytes, 71% of intermediate monocytes, 72.5% of neutrophils, and 77% of macrophages) and was rarely identified with immune cohorts canonically expressing low levels of FcRs. RHBIG-1 induced human FcR signaling in vitro and interacted with FcR-expressing human immune cell cohorts in vitro, suggesting that RHBIG-1 is capable of inducing ADCC and ADCP activity in vivo.

Evaluation of in vivo neutralization of live HBV and HBsAg by RHBIG-1 was measured in murine models simulating prophylactic protection and chronic HBV scenarios. The ability to bind live HBV and prophylactically prevent infection was measured in a human hepatocyte engraftment model (PXB mice) where RHBIG-1 was delivered intravenous (IV) prior to IV inoculation with 1×105 copies of live HBV; protection and prevention of infection resulted in no presence of serum biomarkers of HBV-DNA and HBsAg up to 70 days later. One dose of IV RHBIG-1 at 2 or 0.2 mg/kg was sufficient to prevent infection of engrafted human hepatocytes. In comparison, a 2 mg/kg dose of HyperHEP B failed to prevent HBV infection, as HBV-DNA was detected by day 28; HyperHEP B required a 20 mg/kg dose for prophylactic protection through day 70. Thus, RHBIG-1 was able to bind and neutralize live HBV in serum at a much greater potency than HyperHEP B, as was previously indicated by in vitro experiments.

C57BL/6J mice infected by an adeno-associated virus vector carrying an HBV genomic payload (AAV/HBV) was used to show the dose dependent depletion of serum HBsAg by RHBIG-1 in a chronic HBV model with constitutive HBsAg production. At baseline, serum evaluation was performed to establish circulating levels of HBsAg (~4.33 log IU/mL) and HBV-DNA (~4.25 log copy/μL). One IV dose of RHBIG-1 at 100 mg/kg treatment significantly reduced plasma HBsAg levels on average by −2.8 log through day 1 and by −0.6 log through day 7, and reduced plasma HBV-DNA levels on average by −2.15 log through day 1 and by −0.4 log through day 10, as compared to baseline. One IV dose of RHBIG-1 at 10 mg/kg significantly reduced plasma HBsAg on average by −1.15 log and HBV-DNA levels on average by −1.45 log through day 1 after dosing, as compared to baseline. One IV dose of HyperHEP B at 100 mg/kg did not significantly reduce either HBsAg or HBV-DNA levels compared to baseline at any time point. A second study in the AAV/HBV mouse model replicated these results in male mice as well as in female mice. Together these in vivo data demonstrate that RHBIG-1 can bind and neutralize live HBV and HBsAg in serum at far greater potency than HyperHEP B.

Formal safety pharmacology evaluations (including respiratory, central nervous system, and cardiovascular) were conducted. A preliminary evaluation of safety was performed in the AAV/HBV model, where dosing male and female mice with one IV dose of RHBIG-1 (up to 100 mg/kg) did not induce biological changes to complete blood counts (CBC), concentrations of liver and kidney serum proteins used as organ health readouts, or altered coagulation time or complement activation cascades. Pathology evaluation of liver and kidney tissue 1, 3, 7, 14, and 28 days after IV dosing did not reveal any histopathological changes attributable to test article dosing; inflammatory cell infiltration and necrosis were equivalent across phosphate-buffered saline (PBS) control and test article dosed groups, indicating that any observed histopathological changes were a result of the AAV/HBV infection model itself.

In summary, RHBIG-1 more potently binds HBsAg to neutralize live HBV virus both in vitro and in vivo as compared to HyperHEP B, and more effectively induces FcR-mediated effector function signaling. Although the neutralizing potency of RHBIG-1 aligns with an anti-HBsAg mAb, RHBIG-1 was able to bind more clinically relevant HBsAg escape mutations than the mAb. Furthermore, RHBIG-1 showed efficacy against multiple genotypes of HBV. In concert with nonclinical pharmacokinetics (PK) and toxicology studies, these data support advancement of RHBIG-1 into clinical studies in subjects with chronic HBV infection.

As RHBIG-1 bound specifically to HBsAg, an exogenous antigen, the non-clinical pharmacodynamic efficacy model was selected to be a C57BL/6J mouse infected with AAV/HBV that constitutively produces serum HBsAg. Administration of up to 100 mg/kg of RHBIG-1 in a single IV dose was tolerated by AAV/HBV infected C57BL/6J mice with no negative impact on health biomarkers or through tissue pathology. RHBIG-1 exhibited a dose-dependent depletion of serum HBsAg in vivo that was conserved across gender and with far greater efficacy than HyperHEP B.

In summary, RHBIG-1 potently binds HBsAg to neutralize live HBV virus and prevent viral entry. RHBIG-1 more effectively induces FcR mediated signaling than HyperHEP B and interacts with FcR-expressing human immune cohorts from whole blood. In vivo, RHBIG-1 significantly reduces serum HBsAg level in a dose dependent manner, with far greater efficacy than HyperHEP B. RHBIG-1 does not elicit safety or tolerability signals when administered in an antigenic murine system and does not result in toxicity when administered to Sprague Dawley rats with single or multiple doses up to 500 mg/kg per week. In concert with nonclinical PK and toxicology studies, these data support advancement of RHBIG-1 into clinical studies in subjects with chronic HBV infection.

### 8.3. Example 3: Evaluating Tissue Cross Reactivity (TCR) of rHBIG-1 (Non-Clinical Toxicology Studies)

A toxicology study included an in vitro study that evaluated potential tissue cross reactivity (TCR) of rHBIG-1 in cryosections from a full panel of normal human, non-human primate (NHP) (cynomolgus monkey), and rat (Sprague Dawley) tissues and a 32-day repeat dose toxicity and toxicokinetic (TK) study with a 4 week reversal phase in Sprague Dawley rats.

The results of the TCR study indicated no specific risk for potential organ toxicity in vivo, as expected since HBV is an exogenous target with no known homology to human or mammalian species. Thus, the Sprague Dawley rat was selected as the appropriate species for conducting the repeat dose intravenous (IV) toxicity study. The IV route of administration was used as that is the intended clinical route.

The nonclinical toxicology studies were performed in compliance with International Conference on Harmonization (ICH) M3(R2) and ICH S6(R1), and in compliance with United States FDA (US FDA) Good Laboratory Practices (GLP) regulations.

A summary of nonclinical toxicology studies conducted with RHBIG-1 is presented in Table 13.

TABLE 13
Repeat dose toxicity studies

| | | Species/strain | | Dose | | GLP |
|---|---|---|---|---|---|---|
| Study Number | Study Title | (sex) | Route | (mg/kg/dose) | Test Article | Status |
| rHBIG-1 | A 32-day | Hsd:Sprague | IV | 50, 150 and | RHBIG-1 | Yes |
| | weekly | Dawley SD rats | (slow | 500 mg/kg/week | | |
| | intravenous | (male and | bolus) | (total of 5 | | |
| | injection | female) | | doses) | | |
| | toxicity and | | | | | |
| | toxicokinetics | | | | | |
| | study with | | | | | |
| | rHBIG-1 in | | | | | |
| | Sprague Dawley | | | | | |
| | rats with a 4- | | | | | |
| | Week recovery | | | | | |

Other toxicity studies

| | | | | | | |
|---|---|---|---|---|---|---|
| rHBIG-1 | A Tissue Cross | Tissues from | In | 5 and 10 μg/mL | RHBIG-1 | Yes |
| | Reactivity study | humans, | vitro | | (PN-6091.17) | |
| | with rHBIG-1 | cynomolgus | | | | |
| | in normal | monkeys, and | | | | |
| | human, | Sprague | | | | |
| | cynomolgus | Dawley rats | | | | |
| | monkey, and rat | | | | | |
| | tissue | | | | | |

Abbreviations:
GLP = Good Laboratory Practice;
IV = intravenous

### 8.3.1. rHBIG-1 Repeat Dose Toxicity

A GLP 32-day repeat dose toxicity study in Sprague-Dawley rats with a 4-week recovery was completed with rHBIG-1 and is summarized below.

A 32-Day Weekly Intravenous Injection Toxicity and Toxicokinetic Study in Sprague Dawley Rats with a 4-Week Recovery:

The objective of this GLP study was to determine the potential toxicity and TK of HBIG, when administered once weekly for 5 weeks by IV (slow bolus) injection at dose levels of 50, 150, or 500 mg/kg/week to Sprague Dawley rats, and to assess the reversibility, persistence, or delayed occurrence of toxic effects following a 4-week recovery period. In addition, TK of rHBIG-1 and potential effects on behavior (functional observational battery; FOB) and respiration (plethysmography) were determined.

Male and female Sprague Dawley (Hsd:Sprague Dawley SD) rats were assigned to four groups, and doses were administered as indicated in Table. Animals were dosed via IV (slow bolus) injection once weekly (on Days 1, 8, 15, 22, and 29 of the dosing phase) at a volume of 16.67 mL/kg. The vehicle control article was 20 mM sodium acetate, 270 mM sucrose, 0.02% polysorbate 20 pH 4.8.

The study design is shown below in Table.

TABLE 14
HBIG: GLP 32-Day Repeat-Dose IV Toxicology and
Toxicokinetic Study with a 4-Week Recovery

| | | | Nominal Dose | Number of | |
|---|---|---|---|---|---|
| | | Nominal Dose level^{b} | Concentration^{b} | Animals^{c} | |
| Group^{a} | Subgroup | (mg/kg/week) | (mg/mL) | Males | Females |
| 1 (Control) | 1 (Toxicity) | 0 | 0 | 15 | 15 |
| | 2 (Toxicokinetic) | 0 | 0 | 3 | 3 |
| 2 (Low) | 1 (Toxicity) | 50 | 3 | 15 | 15 |
| | 2 (Toxicokinetic) | 50 | 3 | 6 | 6 |
| 3 (Intermediate) | 1 (Toxicity) | 150 | 9 | 15 | 15 |
| | 2 (Toxicokinetic) | 150 | 9 | 6 | 6 |
| 4 (High) | 1 (Toxicity) | 500 | 30 | 15 | 15 |
| | 2 (Toxicokinetic) | 500 | 30 | 6 | 6 |

Note:
Day 1 of the dosing phase was defined as the first day of dosing for each animal.
^{a}Group 1 was administered vehicle control article only.
^{b}Animals were dosed at a volume of 16.67 mL/kg on Days 1, 8, 15, 22, and 29.
^{c}Toxicity animals: 10 animals/sex/group were designated as terminal animals, and 5 animals/sex/group were designated as recovery animals.

Criteria for evaluation included viability (morbidity/mortality), clinical observations, body weight, food consumption, ophthalmic examinations, FOB (main study females only) and respiratory (main study males only) assessments, clinical pathology (hematology, coagulation, serum chemistry, urinalysis), cytokine analysis, gross (necropsy) evaluation, organ weight, histopathological evaluation, and TK.

All animals survived until scheduled termination. There were no rHBIG-1 related body weight changes; food consumption changes; clinical, ophthalmic, or neurologic observations; tidal volume, respiration rate, or minute volume changes; coagulation or urinalysis parameter changes; organ weight changes or macroscopic findings were noted.

rHBIG-1-related hematology effects included lower red blood cell (RBC) count (−4% to −6%) and hemoglobin concentration (−2% to −3%) and higher mean corpuscular volume (3%), mean corpuscular hemoglobin (2% to 3%), and red cell distribution width (RDW; 5% to 6%) in females administered ≥50 mg/kg/week and minimally higher RDW in males (3% to 6%) administered ≥50 mg/kg/week. These changes were of a small magnitude and may have reflected a subtle increase in RBC turnover. rHBIG-1-related clinical chemistry effects included minimally higher total protein (TP) and globulin (GLOB) concentrations resulting in lower albumin:globulin (A/G) ratio noted in males administered 500 mg/kg/week (TP: 5%; GLOB: 24%, A/G ratio: −21%) and females administered ≥50 mg/kg/week (TP: 3% to 8%; GLOB: 17% to 42%; A/G ratio: −12% to −26%). All rHBIG-1-related clinical pathology effects exhibited reversibility at the end of the recovery phase.

rHBIG-1-related, dose-dependent, 2.2-fold to 6.7-fold increases across both sexes in keratinocyte chemoattractant/human growth-regulated oncogene (KC/GRO) levels were noted at approximately 4 hours postdose on Day 1 of the dosing phase in animals administered ≥50 mg/kg/week, compared with controls. Increased KC/GRO on Day 1 of the dosing phase was transient, and values declined by Day 29 of the dosing phase in animals administered ≥50 mg/kg/week, with levels comparable with or slightly lower than controls. No rHBIG-1-related changes in the levels of TNF-α and no changes in the levels of IFN-γ, IL-1β, IL-4, IL-5, IL-13, IL-6, or IL-10 were noted.

There were no rHBIG-1 related gross pathologic findings or organ weight changes. RHBIG-1-related microscopic findings were limited to the tail IV injection site of females administered 150 or 500 mg/kg/week (slight subcutaneous (SC) fibrosis in females administered 500 mg/kg/week and minimal to slight SC mixed cell infiltrates in females administered ≥150 mg/kg/week). These findings were not noted in recovery phase animals, indicating a full reversal.

The no observed adverse effect level (NOAEL) is 500 mg/kg/week. Following multiple doses on Day 29, this dose level corresponded to mean maximum concentration (Cₘₐₓ) and area under the concentration versus time curve from 0 to 168 hours (AUC₀₋₁₆₈) values of 15200000 ng/mL and 782000000 h*ng/mL, respectively, for combined sexes.

### 8.3.1.1. Local Tolerance

Local tolerance was evaluated in the context of the rHBIG-1 GLP a 32-day repeat dose rat toxicity study by evaluating the IV injection sites. In this study, IV dosing with rHBIG-1 resulted in minimal to slight SC fibrosis and SC mixed cell infiltrates at the injection site at a higher incidence than controls, but these changes were reversible and not considered adverse.

### 8.3.2. rHBIG-1 Other Toxicity Studies

The in vitro study that evaluated potential TCR of rHBIG-1 in cryosections from a full panel of normal human, NHP (cynomolgus monkey), and rat (Sprague Dawley) tissues is summarized below.

8.3.2.1. rHBIG-1: A Tissue Cross Reactivity (TCR) Study in Normal Human, Cynomolgus Monkey, and Rat Tissues:

The objective of this GLP study was to determine the potential cross-reactivity of rHBIG-1 in cryosections from a full panel of normal human, NHP (cynomolgus monkey), and rat (Sprague Dawley) tissues (3 donors/animals per tissue, where available).

A full panel of tissues from each species was incubated with rHBIG-1 at 5 or 10 μg/mL. The study also included isotype control human IgG1 (HuIgG1) as negative control group along with secondary antibody biotinylated F(ab′)₂ donkey anti-human IgG, Fcγ fragment-specific (DkaHuIgG) assay control. In addition, human hypercalcemia of malignancy peptide, amino acid residues 1-34, ultraviolet (UV)-resin spot slides, and cryosections of Chinese hamster ovary (CHO) cells (cell line CSS-1286), were used as negative control tissues and recombinant HBsAg UV-resin spot slides and cryosections of HBsAg-expressing CHO cells (cell line CSS-2474) were used as positive control tissues.

The control HuIgG1 antibody and positive and negative control tissues stained as expected, demonstrating that the assay was sensitive, specific, and reproducible.

In the human prostate, rHBIG-1 weakly to strongly stained the cytoplasm of occasional stromal spindle cells in 1 of 3 donors at 10 μg/mL, with a reduction in the staining to weak to moderate and rare at 5 μg/mL. No other binding was present with rHBIG-1 in the human tissue panel.

In the cynomolgus monkey spinal cord, rHBIG-1 weakly to moderately stained the cytoplasm of rare glial cells and processes at the glia limitans in 1 of 3 animals at 10 μg/mL only. No other binding was present with rHBIG-1 in the cynomolgus monkey tissue panel.

In the Sprague Dawley rat, rHBIG-1 stained the cytoplasm of mucosal epithelial cells in the Fallopian tube and crypt epithelial cells in the large intestine. The Fallopian tube staining was present in 1 of 2 animals and was weak to moderate and occasional at 10 μg/mL and weak and rare at 5 μg/mL. The intestinal staining was observed in 1 of 3 animals and was weak and rare at 10 μg/mL only. No other binding was present with RHBIG-1 in the Sprague Dawley rat tissue panel.

All binding with rHBIG-1 was cytoplasmic in nature, and antibody binding to cytoplasmic sites in TCR studies generally is considered of little to no toxicologic significance due to the limited ability of antibody drugs to access the cytoplasmic compartment in vivo.

### 8.3.3. Conclusions

The toxicology study for rHBIG-1 included an in vitro study that evaluated potential TCR of rHBIG-1 in cryosections from a full panel of normal human, NHP (cynomolgus monkey), and rat (Sprague Dawley) tissues and a 32-day repeat dose toxicity and toxicokinetic study with a 4-week reversal phase in Sprague Dawley rats. The results of the TCR study indicate no specific risk for potential organ toxicity in vivo, as expected since HBV is an exogenous target with no known homology to human or mammalian species. Thus, the Sprague Dawley rat was selected as the appropriate species for conducting the repeat dose IV toxicity study. The IV route of administration was used as that is the intended clinical route.

In the TCR study with a full panel of normal human, cynomolgus monkey, and Sprague Dawley rat tissues, all binding with rHBIG-1 was cytoplasmic in nature.

The administration of rHBIG-1 once weekly to Sprague Dawley rats for 5 doses by IV injection at dose levels up to 500 mg/kg/week was well tolerated.

In conclusion, the nonclinical toxicology studies support advancement of rHBIG-1 into clinical studies.

### 8.4. Example 4: Pharmacokinetic Studies of rHBIG-1

rHBIG-1 described in Examples 1-3 was then tested to characterize the PK of rHBIG-1.

Multiple single- and repeat-dose pharmacokinetic (PK), toxicokinetic (TK), and pharmacology studies were conducted in nonclinical species such as cynomolgus monkeys, mice, and rats to characterize the PK parameters of RHBIG-1, project human PK parameters, and estimate the human equivalent dose (HED) to inform dose selection for first in human (FIH) Study GC2301.

Specifically, single-dose studies were conducted in mice and cynomolgus monkeys to characterize the PK of rHBIG-1. A repeat-dose toxicity study in Sprague-Dawley rats was also completed. The Sprague-Dawley rat TK data were used to project human PK parameters using a 2-compartment model, assuming linear PK and target-independent clearance. Additionally, rHBIG-1 efficacy data from in vivo efficacy/PK-PD study in an immune competent chronic AAV/HBV infection mice model were integrated to further project a pharmacologically active dose/exposure in humans.

Following a single dose IV administration of 285 IU/kg with rHBIG-1 (2.9 mg/kg) to two non-naïve cynomolgus non-human primates (NHP) monkeys, a biphasic elimination pattern in NHP typically exhibited by systemically administered monoclonal antibodies (mAbs) was observed.

Administration of 285 IU/kg (2.9 mg/kg) rHBIG-1 resulted in mean maximum concentration (Cₘₐₓ) of 3672 mIU/mL with the mean time to maximum concentration (Tₘₐₓ) reached at 4.5 hours. The mean area under concentration curve (AUC) from 0 to infinity (AUC_{0-∞}) value was 1280000 h*mIU/mL. The mean serum clearance (CL) was 0.0122 L/day (range: 0.0109 L/day to 0.0135 L/day), volume of distribution (V or Vz) was 0.302 L (range: 0.273 L to 0.330 L), and mean terminal half-life (t1/2) was 17.5 days (range: 14 days to 21 days). The calculated mean rHBIG-1 CL, Vz and t_{1/2} were similar to human IgG1 mAb PK parameters reported for cynomolgus monkeys in literature. In this NHP study, rHBIG-1 induced no signs or symptoms of over toxicity in any of the animals.

Following a single IV bolus administration of rHBIG-1 at 10, 30, or 100 mg/kg to C57BL/6J mice, serum rHBIG-1 concentrations peaked at 0.50 h for the 10 and 30 mg/kg dose groups and 4.00 h for the 100 mg/kg dose group (as values at 0.50 h were all above the limit of quantitation [ALQ]). Serum rHBIG-1 concentrations decreased in a multi-exponential manner. Cₘₐₓ values for the 10, 30, and 100 mg/kg rHBIG-1 treatment groups were 399000, 1080000, and 3060000 ng/mL, respectively. AUC from time zero to 168 h (AUC₀₋₁₆₈) values for the 10, 30, and 100 mg/kg rHBIG-1 treatment groups were 25300000, 68500000, and 168000000 h*ng/mL, respectively. AUC from time zero to 672 h (AUC₀₋₆₇₂ or AUC₀₋ₜ) values were 65800000, 172000000, and 322000000 h*ng/mL for the 10, 30, and 100 mg/kg rHBIG-1 treatment groups, respectively. T_{1/2}, AUC_{0-∞}, C_{L}, and V for the 10 mg/kg rHBIG-1 treatment group was 468 hours, 109000000 h*ng/mL, 0.0916 mL/h/kg, and 63.5 mL/kg, respectively. Due to an inability to characterize a terminal elimination phase, estimation of elimination phase t_{1/2} was not possible at 30 and 100 mg/kg dose levels. Overall, exposures appeared to increase generally in a less than dose proportional manner for C₀, Cₘₐₓ, AUC₀₋₁₆₈, AUC₀₋₆₇₂ and AUC₀₋ₜ over the dose range tested. The administration of 10, 30 or 100 mg/kg IV single doses RHBIG-1 in healthy male C57BL/6J mice was well tolerated and safe.

The efficacy of rHBIG-1 in a mouse model of HBV infection was evaluated in AAV/HBV infected male C57BL/6J mice treated post-infection Day 28 with vehicle/phosphate buffer saline (PBS) or IV 10 mg/kg or 100 mg/kg single doses of rHBIG-1. Administration of 10 mg/kg and 100 mg/kg rHBIG-1 resulted in mean Cₘₐₓ of approximately 113000 ng/mL and 173000 ng/mL, respectively with the mean Tₘₐₓ reached at approximately 0.25 hours, first timepoint collected post injection. The mean AUC_{0-∞} values were 366000 h*ng/mL and 4040000 h*ng/mL following single dose administration of 10 mg/kg and 100 mg/kg of rHBIG-1 respectively. The male C57BL/6J mice were infected with AAV/HBV 28 days prior to rHBIG-1 dosing to ensure establishment of HBV infection and chronicity of infection. Prior to RHBIG-1 dosing at Day 0, baseline measurements of serum HBsAg was taken at Days −11 and −4 for inter-mouse tracking of changes to HBsAg following rHBIG-1 dosing. Once established HBV infection was confirmed, mice were intravenously dosed with 10 mg/kg and 100 mg/kg of rHBIG-1 on Day 0 of the study. The blood plasma samples (n=3 animals/timepoint/group) were collected at 15 min, 30 min, 1 hr, 4 hr, 8 hr, 1, 2, 7, 10, 21, 28 and 35 days post dose to quantitatively analyze HBsAg and unbound rHBIG-1 levels. For each blood draw listed, the HBsAg titer was measured.

Plasma analysis for RHBIG-1 was performed using a validated ELISA for anti-HBs. The PK parameters were determined using Phoenix WinNonlin version 8.4.0 software (Certara USA, Inc. Princeton, New Jersey) using a non-compartmental approach. Administration of 10 mg/kg and 100 mg/kg rHBIG-1 resulted in mean Cmax of approximately 113000 ng/mL and 173000 ng/mL, respectively with the mean Tmax reached at approximately 0.25 hours, the first timepoint collected post injection. The mean AUC0-∞ values were 366000 h*ng/mL and 4040000 h*ng/mL following single dose administration of 10 mg/kg and 100 mg/kg of RHBIG-1 respectively. Pharmacokinetic evaluation suggests rHBIG-1 exhibited AUC and Cmax slightly greater than proportional between 10 mg/kg and 100 mg/kg and the t1/2 estimate was approximately 205 hours (9 days) following 100 mg versus approximately 163 hours (7 days) following 10 mg/kg suggestive of TMDD.

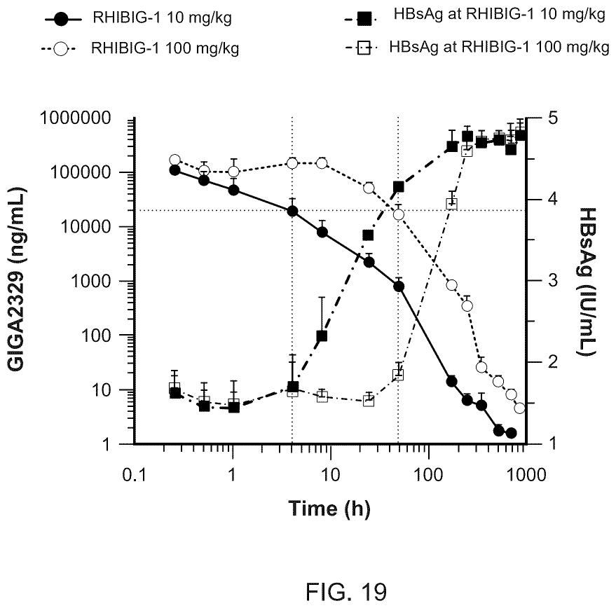

rHBIG-1 potently neutralized HBsAg at both active doses dropping post-administration HBsAg concentrations >3 log units from baseline. Suppression of HBsAg was maintained while rHBIG-1 concentrations remained at or above approximately 20,000 ng/mL, independent of the dose, at which point, the HBsAg increased, returning to baseline. These data indicated maximal efficacy is observed at a rHBIG-1 serum concentration of approximately 20,000 ng/mL (FIG. 19) i.e., rHBIG-1 serum concentration of approximately 20,000 ng/mL are required to suppress HBsAg.

The administration of 10 mg/kg or 100 mg/kg IV single doses rHBIG-1 in AAV/HBV infected male C57BL/6J mice treated post-infection Day 28 was well tolerated and safe.

Pharmacokinetic evaluation suggested rHBIG-1 exhibited AUC and Cmax slightly greater than proportional between 10 mg/kg and 100 mg/kg and the t1/2 estimate was approximately 205 hours (9 days) following 100 mg versus approximately 163 hours (7 days) following 10 mg/kg suggestive of target mediated drug disposition (TMDD). rHBIG-1 potently neutralized HBsAg at both active doses dropping post-administration HBsAg concentrations >3 log units from baseline. Suppression of HBsAg was maintained while rHBIG-1 concentrations remained at or above approximately 20,000 ng/mL, independent of the dose, at which point, the HBsAg increased, returning to baseline. These data indicate maximal efficacy is observed at a rHBIG-1 serum concentration of approximately 20,000 ng/mL i.e., RHBIG-1 serum concentration of approximately 20,000 ng/mL are required to suppress HBsAg. The administration of 10 mg/kg or 100 mg/kg IV single doses rHBIG-1 in AAV/HBV infected male C57BL/6J mice treated post-infection Day 28 was well tolerated and safe.

As part of a GLP toxicity study, 50, 150 and 500 mg/kg of rHBIG-1 was administered weekly via IV (slow bolus) injection for at least 32 days (total of 5 doses) to Sprague Dawley rats. The TK characteristics of rHBIG-1 were determined following a single and repeated slow bolus weekly IV injection in Sprague Dawley rats on Days 1 and 29. Serum analysis for rHBIG-1 was performed using a validated enzyme immunoassay for hepatitis B surface antibodies (anti-HBs). Following injection of rHBIG-1 serum concentrations peaked, as expected, at the first timepoint sampled post injection (1.00 hour) and decreased in a biexponential manner. For all animals, mean concentrations generally stayed above lower limit of quantitation (LLOQ) up to the last sampling timepoint across dose levels. There was no apparent sex difference, exposure generally increased in a dose proportional manner across all the tested dose levels for Cₘₐₓ and C₀. For AUC₀₋₁₆₈ and AUC₀₋₆₇₂ (Day 29) the increase in exposure appeared to be less than dose proportional for the doses tested. There was minor accumulation upon repeated dosing on Day 29. Consistent with the observed t1/2 (1-2 weeks), steady state was apparently not achieved by Day 29. Otherwise, CL and V appeared consistent with large molecule kinetics.

In summary, the PK parameters of rHBIG-1 were evaluated in a series of single- and repeat-dose studies in nonclinical species. Following IV bolus administration of RHBIG-1 in cynomolgus monkeys, mice, and rats, a biphasic (multi-exponential in mice) elimination pattern typically exhibited by systemically administered mAbs was observed. The calculated mean rHBIG-1 PK parameters CL, Vz and t_{1/2} were similar to human IgG1 mAb PK parameters reported for cynomolgus monkeys and rats in literature. In rats, peak exposure generally increased in a dose proportional manner whereas systemic exposure increase appeared to be less than dose proportional for the doses tested. In rats, minor accumulation was observed upon repeated dosing.

The Sprague-Dawley rat TK data were used to project human PK parameters using a 2-compartment model, assuming linear PK and target-independent clearance. These projections were integrated with rHBIG-1 efficacy data from an in vivo efficacy/PK-PD study in an immune competent chronic AAV/HBV infection mice model. rHBIG-1 potently neutralized HBsAg at both active doses dropping post-administration HBsAg concentrations >3 log units from baseline. These data indicate maximal efficacy is observed at a rHBIG-1 serum concentration of approximately 20,000 ng/mL i.e., rHBIG-1 serum concentration of approximately 20,000 ng/mL are required to suppress HBsAg.

TABLE 15
Summary of nonclinical pharmacokinetic and toxicokinetic studies for rHBIG-1.

| | | | | | |
|---|---|---|---|---|---|
| Study Number | Study Title | Species/strain (Sex) | Route | Dose | Test Article |

Single-Dose PK Studies

| | | | | | |
|---|---|---|---|---|---|
| rHBIG-1_{—} | Pharmacokinetic/tolerability safety | Cynomolgus NHP | IV bolus | 2.9 mg/kg (285 IU/kg) for | rHBIG or |
| PK_001 | study in Non-Human Primates (NHP) | (Macaca fascicularis) | | RHBIG-1 (PN-2389); 260 mg/k [symbol] | HyperHEP B |
| | using an early R&D lot of RHBIG-1 | (male) | | (285 IU/kg) | (PN-1020) |
| | | | | HyperHep B | |
| rHBIG-1_{—} | An in vivo assessment of the | C57 Black | IV bolus and | 10, 30 and 100 mg/kg single | rHBIG or |
| PK_002 | pharmacokinetics of RHBIG-1 followi [symbol] | (C57BL/6NHsd) | SC | dose for RHBIG-1 IV; | HyperHEP B |
| | single-dose intravenous and | mice (male) | | 30 and 100 mg/kg single dose | (H01H051313) |
| | subcutaneous administration to male | | | for RHBIG-1 SC; 100 mg/kg | |
| | C57BL/6 mice | | | single dose for HyperHep B IV | |

Repeat-Dose TK Study

| | | | | | |
|---|---|---|---|---|---|
| rHBIG-1_{—} | A 32-day weekly intravenous | Hsd:Sprague | IV | 50, 150 and 500 mg/kg/week (t [symbol] | rHBIG |
| TOX_002 | injection toxicity and toxicokinetic stu [symbol] | Dawley SD rats (male) | (slow bolus) | of 5 doses) | |
| | with RHBIG-1 in Sprague Dawley rats | | | | |
| | with a 4-Week recovery | | | | |

Nonclinical in vivo Pharmacology Study (for Potential Efficacious Human Dose Projection)

| | | | | | |
|---|---|---|---|---|---|
| rHBIG-1_{—} | In vivo evaluation of pharmacodynami [symbol] | C57BL/6J mice (male) | IV injection | 10 and 100 mg/kg single dose | rHBIG |
| VIV_004^{#} | and tolerability of RHBIG-1 in an | | | | |
| | immune competent chronic | | | | |
| | AAV/HBV infection model | | | | |

Abbreviations: GLP = Good Laboratory Practice; IV = intravenous; IU = International Unit; PK = Pharmacokinetics; SC = Subcutaneous; TK = Toxicokinetics. [symbol]
indicates data missing or illegible when filed

### Methods of Analysis:

Quantitation of rHBIG-1 in Serum:

Enzyme-linked immunosorbent assay (ELISA) methods were utilized to measure rHBIG-1 concentrations in serum samples collected during the non-GLP PK study in C57BL/6 mice and the GLP TK study in Sprague Dawley rats.

Quantitation of rHBIG-1 in Plasma:

Enzyme-linked immunosorbent assay (ELISA) methods were utilized to measure rHBIG-1 concentrations in plasma samples collected during the pharmacodynamics (PD) and tolerability study in AAV/HBV infected C57BL/6 mice and the PK study in cynomolgus NHPs.

### Quantitation of HyperHEP B in Serum:

Enzyme-linked immunosorbent assay (ELISA) methods were utilized to measure HyperHEP B concentrations in serum samples collected during a non-GLP PK study in C57BL/6 mice.

### 8.5. Example 5: Phase 1 Clinical Study Evaluating Safety, Tolerability, and Pharmacokinetics of rHBIG-1 Administered as a Single Ascending Dose and Multiple Ascending Doses in Human Subjects with Chronic Hepatitis B Virus Infection

This study is a randomized, placebo controlled, double-blind, Phase 1 study that is conducted in 2 parts for evaluating safety, tolerability, and pharmacokinetics of rHBIG-1 administered as a single ascending dose and multiple ascending doses in human subjects with chronic Hepatitis B Virus Infection.

RHBIG-1 is a fully human pAb (IgG1/IgK) that binds to multiple HBsAg variants to neutralize and potentially clear the virus and viral particles in patients with HBV infection. It is administered via IV infusion.

RHBIG-1 is a sterile liquid formulation of recombinant immunoglobulins. RHBIG-1 is a clear to slightly opalescent, colorless to slightly brown solution contained in a 5.0 mL solution of immunoglobulins in a 10 mL vial.

Part 1 utilizes a single ascending dose (SAD) schema while Part 2 utilizes a multiple ascending dose (MAD) schema. The study evaluates the safety, tolerability, pharmacokinetics (PK), and pharmacodynamics (PD) of IV-administered RHBIG-1, a recombinant human pAb for HBV infection, in participants with chronic HBV B infection.

### 8.5.1. Part 1

The primary objective in Part 1 is to evaluate the safety and tolerability of a single IV dose of RHBIG-1 in participants with chronic HBV infection. The secondary objective in Part 1 is to characterize the PK of RHBIG-1 following a single IV dose in participants with chronic HBV infection. The exploratory objective in Part 1 is to evaluate the pharmacodynamic (PD) effects of RHBIG-1 following a single IV dose in participants with chronic HBV infection.

In Part 1 (SAD) of the study, adult participants with chronic HBV infection are enrolled into respective escalating cohorts to receive a single intravenous (IV) dose of RHBIG-1 or placebo on Day 1 and be followed for a total of 105 days after dosing. There are 4 cohorts consisting of 8 participants each. In each cohort, the 8 participants receive either RHBIG-1 or placebo, such that 6 participants receive RHBIG-1 and 2 participants receive placebo. In Part 1, 21 mg (Cohort 1), 70 mg (Cohort 2), 210 mg (Cohort 3), and 700 mg (Cohort 4), of RHBIG-1 diluted with saline (0.9% Sodium Chloride Injection USP) is administered as a single IV infusion on Day 1.

For each dose level, two sentinel participants initially are randomized in 1:1 ratio (active:placebo) such that 1 participant is randomly assigned to receive the active drug whilst the other receives placebo. The sentinel participants are dosed, and followed for 24 hours to assess safety and tolerability. If no safety issues arise, the remaining 6 participants in the cohort are randomized 5:1 to RHBIG-1:placebo.

The primary safety endpoints in Part 1 include:
Incidence and severity of treatment-emergent AEs (TEAEs) and serious AES (SAEs).
Changes from baseline in laboratory parameters (hematology, clinical chemistry, complement component 3 [C3], complement component 4 [C4], and urinalysis).
Changes in vital signs (blood pressure, heart rate, and temperature).
Changes in electrocardiograms (ECGs).

In Part 1, the secondary endpoints include characterizing the following PK parameters:
Maximum plasma concentration (Cmax).
Area under the concentration time curve from 0 to the last quantifiable concentration(AUC0-t).
Dose normalized area under the concentration curve from 0 to infinity (AUC0-∞),
Dose normalized maximum plasma concentration (DN_Cmax).
Dose normalized area under the concentration time curve from 0 to the last quantifiableconcentration (DN_AUC0-t).
Dose normalized area under the concentration curve from 0 to infinity (DN_AUC0-∞).
Time to obtain maximum concentration (Tmax).
Terminal half-life (t1/2).
Volume of distribution (Vz).
Clearance (CL).

### 8.5.2. Part 2 Multiple Ascending Dose (MAD)

The primary objective in Part 2 is to evaluate the safety and tolerability of a multiple IV dose of RHBIG-1 in participants with chronic HBV infection. The secondary objective in Part 2 is to characterize the PK of RHBIG-1 following multiple IV doses in participants with chronic HBV infection. The exploratory objective in Part 2 is to evaluate the PD effects of RHBIG-1 following multiple IV doses in participants with chronic HBV infection.

In Part 2 (MAD) of the study, adult participants with chronic HBV infection are enrolled and receive 6 doses, one every 4 weeks (Q4W), of RHBIG-1 or placebo. A starting dose for the initial cohort in Part 2 (MAD) is informed by the Part 1 (SAD) interim data analysis and PK/PD modeling. A staggered approach is employed where initiation of the next MAD cohort occurs following a recommendation from the SRC in Part 2. There are at least two cohorts for Part 2 (MAD). Participants in each MAD cohort are followed for 105 days after the last dose, for an overall duration of approximately 245 days. In each MAD cohort, 8 adult participants with chronic HBV are randomized 3:1 to receive either RHBIG-1 or placebo, such that 6 participants receive RHBIG-1 and 2 participants receive placebo.

The primary safety endpoints include:
Incidence and severity of treatment emergent AEs and SAEs.
Changes from baseline in laboratory parameters (hematology, clinical chemistry, C3, C4 and urinalysis).
Changes in vital signs (blood pressure, heart rate and temperature).
Changes in ECGs.
Secondary endpoints in Part 2 include characterizing the following PK parameters:
Plasma concentration at the end of the dosing interval (Ctrough).
Area under the plasma concentration versus time curve during the dosing interval (AUC0-tau).
Cₘₐₓ
AUC₀₋ₜ
AUC_{0-∞}
Accumulation ratio maximum plasma concentration (ARCmax).
Accumulation ratio area under the concentration curves (ARAUCs).
Tₘₐₓ
t_{1/2}
V_{z}
C_{L}

### 8.5.3. Diagnosis and Main Criteria for Inclusion:

### Inclusion Criteria:

A participant must meet all the following inclusion criteria to be eligible for participation in this study.

1. ≥18 years of age at the time of signing the informed consent.

2. HBeAg negative chronic HBV infection for ≥6 months, defined as presence of HBsAgin serum for 6 months.

3. Serum HBsAg concentration between ≥100 IU/mL and ≤2000 IU/mL at screening.

4. Currently on stable dose of NAs (≥6 months) and expected to continue while in study.

5. Serum HBV DNA concentration, ≤50 IU/mL at screening.

6. Agree for the following contraception requirements:

A) A male participant must refrain from donating spermatozoa and agree to use contraception during the treatment period and for at least 90 days following the last dose unless surgically sterile (vasectomy with confirmed absence of spermatozoa). In addition, non-surgically sterile male participants must:

Be abstinent from heterosexual intercourse as their preferred and usual lifestyle(abstinent on a long term and persistent basis) OR use a barrier method of contraception, such as a male condom (and should also be advised of the benefit for a female partner to use a highly effective method of contraception as a condom may break or leak) when having sexual intercourse with a woman of childbearing potential (WOCBP).

B) A female participant is eligible to participate if she is not pregnant or breastfeeding AND at least one of the following conditions applies: not a WOCBP (i.e., postmenopausal [absence of menses for at least 1 year without an alternative cause] or surgically sterile (if confirmed hysterectomy, bilateral sal pingo-oophorectomy, bilateral tubal ligation) OR is a WOCBP and using a contraceptive method that is highly effective (with a failure rate of <1 percent per year, for example; oral, injectable or implanted hormonal methods of contraception, placement of an intrauterine device or intrauterine system, condom or occlusive cap with spermicidal foam/gel/film/cream/suppository), preferably with low user dependency during the intervention period and for at least 90 days after the last dose of study treatment.

A WOCBP must have both a confirmed menstrual period prior to the first dose of study intervention (additional evaluation [e.g., amenorrhea in athletes, birth control] should also be considered) and a negative highly sensitive pregnancy test (urine or serum) within 24 hours before the first dose of study treatment.

### 8.5.4. Exclusion Criteria

A participant meeting any of the following exclusion criteria is NOT eligible for participation in the study.
1. Positive for co-infection with hepatitis C virus (HCV), human immunodeficiency virus(HIV), hepatitis D virus (HDV) at screening.
2. Participants that weigh less than 50 kg.
3. History of documented liver cirrhosis at screening. Patients under liver cirrhosis evaluation at screening are not eligible until cirrhosis is ruled out.
4. Liver stiffness >8 kPa at screening.
5. Malignancy diagnosed and/or treated within 5 years prior to Screening, with the exception of localized non-metastatic basal cell or squamous cell carcinoma of the skinor in-situ carcinoma of the cervix excised with curative intent.
6. Currently taking, or took within 3 months of Day 1, any immunosuppressing drugs (e.g., prednisone), other than a short course of therapy (≤2 weeks) or topical/inhaled steroid use.
7. Participants requiring anti-coagulation therapies (for example warfarin, Factor Xa inhibitors, or anti-platelet agents like clopidogrel).
8. Participation in a clinical trial and has received an investigational product within the following time period prior to the first dosing day in the current study: 5 half-lives (if known) or 2× the duration (if known) of the biological effect of the study treatment (whichever is longer) or 90 days (if half-life or duration is unknown).
9. Male participants with a corrected QT interval using Fridericia's formula (QTcF) >450 msec and female participants with QTcF >470 msec on ECG recorded at screening. Participants with evidence of intraventricular conduction delay, defined as QRS interval greater than 110 msec, is excluded if the QTcF is >500 msec for both males and females.
10. Laboratory results as follows at screening:
   a. Estimated glomerular filtration rate (eGFR)<60 mL/min as calculated by the Chronic Kidney Disease Epidemiology (CKD-EPI) formula at screening.
   b. ALT >1.5×ULN.
   C. International normalized ratio (INR) >1.25.
   d. Platelet count <140×109 cells/L.
   e. Total bilirubin >1.25×ULN (unless attributable to known Gilbert's syndrome with normal direct bilirubin).
11. Participant test positive for amphetamine, cocaine, methamphetamine, methylenedioxymethamphetamine or phencyclidine in urine drug screen performed at Screening, or has a recent history (≤6 months) of alcohol or drug abuse. A participant is not excluded due to positive drug screen caused by prescribed medications.
12. Known hypersensitivity to any RHBIG-1 excipients or a history of drug or other allergy that, in the opinion of the Investigator, contraindicates participation.
13. Used prescription drugs within 14 days before Day 1 except for a stable dose of medication started ≥3 months before screening.
14. Women who are pregnant, planning on becoming pregnant, lactating, or breast-feeding.
15. Any reason that, in the opinion of the Investigator or Medical Monitor, precludes the participant from participating in the study (e.g., any previous or current condition) that may increase the risk associated with study participation or that would confound study analysis or impair study participation or cooperation.
16. Participants who cannot or are unwilling to give independent consent.

### 8.5.5. Part 1 (SAD) Dosing

A single IV dose of RHBIG-1 on Day 1 for Cohort 1 followed by up to 3 additional ascending dose cohorts.

In Part 1, 21 mg (Cohort 1), 70 mg (Cohort 2), 210 mg (Cohort 3), and 700 mg (Cohort 4), of RHBIG-1 is administered as a single IV infusion on Day 1.

### 8.5.6. Part 2 (MAD) Dosing

Participants in Part 2 (MAD) receive Q4W infusions of RHBIG-1 or placebo beginning on Day 1 and continuing for 6 months.

An IV dose of RHBIG-1 at Day 1, Day 29, Day 57, Day 85, Day 113, and Day 141 for the Low Dose Cohort. RHBIG-1 is administered on the same days in the High Dose Cohort.

For Placebo, A matched volume of the respective dose used in each cohort is administered intravenously.

### 8.5.7. Duration of Treatment

Part 1 (SAD): A Screening Period of approximately 30 days, 1 day of treatment, and 105 days of follow-up. The expected duration for participants in Part 1 (SAD) is approximately 5 months (135 days).

Part 2 (MAD) consists of a Screening Period of approximately 30 days, a treatment period of 6 months (monthly RHBIG-1 infusions) and a follow-up period of 105 days. The expected duration for participants is approximately 9 months (275 days). The 6 month duration includes monthly dosing [Q4W].

### 8.5.8. Secondary Pharmacokinetic Analyses:

PK parameters are calculated through non-compartmental (NCA) methods using Phoenix® WinNonlin® software, version 8.3 or later (Certara USA, Inc. [Princeton, NJ]). The following PK parameters are calculated following a single dose of RHBIG-1: maximum plasma concentration (Cₘₐₓ), time to obtain maximum concentration (Tₘₐₓ), area under concentration curve from 0 to infinity (AUC0-∞), area under concentration curve from 0 to the last quantifiable concentration (AUC0-t), terminal half-life (t1/2), systemic clearance (CL), volume of distribution (V_{z}), dose normalized Cₘₐₓ (DN_Cₘₐx), dose-normalized AUC0-∞ (DN_AUC0-∞) and dose-normalized AUC0-t (DN_AUC0-t).

The following steady-state PK parameters of RHBIG-1 from multiple IV administration are estimated: plasma concentration at the end of the dosing interval (C_{trough}), area under the plasma concentration versus time curve during the dosing interval (AUC0-tau), Cₘₐₓ, AUC₀₋ₜ, AUC_{0-∞} accumulation ratio (ARCₘₐₓ, ARAUCs), Tₘₐₓ, t1/2, V_{z} and C_{L}. Additional PK parameters are estimated if data permits.

### 8.5.9. Administration

RHBIG-1 is administered via intravenous (IV) infusion once every 4 weeks. Part 1 of the study is a single ascending dose (SAD) design with 4 planned dosing cohorts. RHBIG-1 formulation is administered as a single IV dose at 21 mg, 70 mg, 210 mg and 700 mg in Cohorts 1, 2, 3 and 4 respectively. Part 2 of the study is a multiple ascending dose (MAD) design, which will include a low and high dose cohort. Participants are randomized to RHBIG-1, or placebo, which is administered every four weeks (Q4W) over a period of 6 months.

### 9. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### 10. EQUIVALENTS

Whereas various specific embodiments have been illustrated and described, the above specification is not restrictive. It will be appreciated that various changes can be made without departing from the spirit and scope of the invention(s). Many variations will become apparent to those skilled in the art upon review of this specification.

1. A pharmaceutical composition comprising a library of antigen-binding proteins (ABPs), wherein the library comprises (i) at least 100 ABPs specifically binding to a Hepatitis B virus (HBV) surface antigen and (ii) a pharmaceutically acceptable excipient, wherein each of the ABPs comprises a cognate pair of heavy chain and light chain variable regions from a single cell out of a blood sample from a donor previously vaccinated with an HBV vaccine; and has been generated from a polynucleotide construct encoding the cognate pair of heavy chain and light chain variable regions.

2. The pharmaceutical composition of claim 1, wherein each of the ABP has been generated by the process of:
a. isolating single cells from the blood sample from the donor previously vaccinated with the HBV vaccine;
b. amplifying polynucleotides, wherein each of the polynucleotides encodes a cognate pair of heavy chain and light chain variable regions from one of the single cells by overlap extension reverse transcriptase polymerase chain reaction (OE-RT-PCR);
c. cloning the polynucleotides obtained from the amplification into an expression vector, thereby obtaining the polynucleotide constructs encoding antibody fragments;
d. generating antibody expression constructs using the polynucleotide constructs or a subset of the polynucleotide constructs, wherein each of the antibody expression constructs encodes a light chain variable region, a kappa or lambda-type light chain constant region, a heavy chain variable region, and a heavy chain constant region,
e. introducing the antibody expression constructs into a cell line, and
f. expressing antibodies from the antibody expression constructs in the cell line.

3. The pharmaceutical composition of claim 1, wherein the donor has been selected as having an anti-HBV antigen serum titer of 50 IU/mL or higher, 100 IU/mL or higher, 150 IU/mL or higher, or 200 IU/mL or higher.

4. (canceled)

5. The pharmaceutical composition of claim 1, wherein the blood sample comprises:
(i) CD27+ cells purified from PBMC of the donor,
(ii) CD43+ cells purified from PBMC of the donor;
(iii) CD27+ cells and CD43+ cells purified from PBMC of the donor;
(iv) CD27+ or CD43+ cells expressing antibodies comprising a IgK light chain; or
(v) CD27+ or CD43+ cells expressing antibodies comprising a IgL light chain.

6-9. (canceled)

10. The pharmaceutical composition of claim 1, further comprising preparing the blood sample by:
(i) isolating CD27+ or CD43+ cells from PBMC of the donor;
(ii) isolating CD27+ or CD43+ cells expressing antibodies comprising a IgK light chain from PBMC of the donor;
(iii) isolating CD27+ or CD43+ cells expressing antibodies comprising a IgL light chain from PBMC of the donor;
(iv) isolating CD27+ cells and CD43+ cells from PBMC of the donor;
(v) isolating CD27+ cells and CD43+ cells expressing antibodies comprising a IgK light chain from PBMC of the donor; or
(vi) isolating CD27+ cells and CD43+ cells expressing antibodies comprising a IgL light chain from PBMC of the donor.

11-15. (canceled)

16. The pharmaceutical composition of claim 1, wherein the donor has been vaccinated with a booster dose of the HBV vaccine, or wherein the donor has been vaccinated with a booster dose of the HBV vaccine and the blood sample was obtained from the donor on day 7-15 or on day 10-12 after the booster vaccination.

17-18. (canceled)

19. The pharmaceutical composition of claim 1, wherein:
(i) the library comprises ABPs originated from two or more donors;
(ii) the library comprises ABPs originated from at least five, at least ten, at least fifteen, at least twenty, at least twenty-five, or at least thirty donors; or
(iii) the library comprises ABPs originated from two, three, four, five, six, seven, eight, nine, ten, eleven, twelve, thirteen, fourteen, fifteen, sixteen, seventeen, eighteen, nineteen, or twenty donors.

20-21. (canceled)

22. The pharmaceutical composition of claim 2, wherein the step a. comprises:
(i) isolating single cells from blood samples from two or more donors vaccinated with an HBV vaccine;
(ii) isolating at least 100 single cells from blood samples from two or more donors vaccinated with the HBV vaccine;
(iii) isolating single cells from blood samples from at least five, at least ten, at least fifteen, at least twenty, at least twenty-five, or at least thirty donors vaccinated with the HBV vaccine; or
(iv) isolating at least 100 single cells from blood samples from at least five, at least ten, at least fifteen, at least twenty, at least twenty-five, or at least thirty donors vaccinated with the HBV vaccine.

23-25. (canceled)

26. The pharmaceutical composition of claim 1, wherein the process further comprises the step of obtaining the subset of the constructs after step c. by expressing the antibody fragments from the constructs and enriching the subset of the constructs based on the binding activities of the antibody fragments against the HBV surface antigen.

27. The pharmaceutical composition of claim 1, wherein the library comprises at least 1000 or at least 10,000 ABPs, or wherein the library comprises at least 500, 1,000, 2,000, 3,000, 4,000, 5,000, 10,000, 100,000, or more unique human ABPs specifically binding to an HBV surface antigen.

28. The pharmaceutical composition of claim 1, wherein each of the ABP is an IgG1 antibody, optionally comprising an IgK light chain.

29. (canceled)

30. The pharmaceutical composition of claim 1, wherein the HBV vaccine in targets hepatitis B surface antigen (HBsAg).

31. The pharmaceutical composition of claim 1, wherein the at least 100 ABPs have one or more of the following features:
(i) a binding EC50 against the HBV surface antigen lower than 1 μg/ml or lower than 0.5 μg/ml, 0.1 μg/ml, 0.05 μg/ml, 0.01 μg/ml, or 0.001 μg/ml;
(ii) at least 100-fold, at least 500-fold, 1,000-fold, 1,500-fold, 2,000-fold, 3,000-fold, 4,000-fold, or 5,000-fold lower binding EC50 against the HBV surface antigen compared to a plasma sample from the donor vaccinated with an HBV vaccine;
(iii) a neutralization IC50 against HBV lower than 0.5 μg/ml, or lower than 0.1 μg/ml, 0.05 μg/ml, 0.01 μg/ml, 0.005 μg/ml, 0.001 μg/ml, 20 ng/ml, or 2.8 ng/ml; or
(iv) at least 100-fold, at least 500-fold 1,000-fold, 2,000-fold, 3,000-fold, 4,000-fold, 5,000-fold, 10,000-fold, 20,000-fold, 30,000-fold, 40,000-fold, or 50,000-fold lower neutralization IC50 against HBV compared to a plasma sample from the donor vaccinated with an HBV vaccine;
wherein the EC50 is measured by ELISA and the IC50 against HBV is measured by detecting neutralization of live HBV infection to human hepatocyte cells.

32-41. (canceled)

42. The pharmaceutical composition of claim 1, wherein:
(i) the polynucleotide construct has been enriched based on binding affinity of a protein encoded by the polynucleotide construct to the HBV surface antigen; or
(ii) the polynucleotide construct has been enriched based on binding affinity of a protein encoded by the polynucleotide construct to a first HBV surface antigen and a second HBV surface antigen; or
(iii) the polynucleotide construct has been enriched based on binding affinity of a protein encoded by the polynucleotide construct to a first HBV surface antigen and a second HBV surface antigen, wherein the first antigen and the second antigen are independently selected from adw, adr, ayw, or ayr antigens.

43-44. (canceled)

45. The pharmaceutical composition of claim 1, which includes one or more of the following technical features:
(i) the pharmaceutical composition further comprises a compound selected from benzalkonium chloride, benzyl alcohol, benzoic acid, salicylic acid, thimerosal, phenethyl alcohol, methylparaben, propylparaben, chlorhexidine, sorbic acid, or hydrogen peroxide in an amount effective to preserve the pharmaceutical composition;
(ii) the pharmaceutical composition is free of preservatives;
(iii) the library includes at least 100 ABPs and is at 10 g/L to 300 g/L, at 10 g/L to 50 g/L, at 10 g/L to 100 g/L, or at 10 g/L to 200 g/L concentration;
(iv) the library includes at least 100 ABPs and is at 10, 20, 30, 40, or 50 g/L concentration;
(v) the pharmaceutical composition further comprises 20 mM acetate, 270 mM sucrose, and 0.2 mg/mL (0.02% w/v) Polysorbate 20, pH 4.8;
(v) the pharmaceutical composition is free of alive or dead HBV or any protein or polynucleotide produced from HBV;
(vi) the pharmaceutical composition has a CEX-HPLC peak distribution having at least 80%, 90%, 95%, 98%, or 99% overlap with the distribution in FIG. 20 in terms of peak sizes, peak retention times, or peak numbers, wherein the CEX-HPLC is performed with a pH gradient from a low pH (pH 5-6) mobile phase A to a high pH (pH 10-11) mobile phase B;
(vii) the pharmaceutical composition has a CEX-HPLC peak distribution having at least 80%, 90%, 95%, 98%, or 99% overlap with the distribution in FIG. 20 in terms of peak sizes, peak retention times, and peak numbers, wherein the CEX-HPLC is performed with a pH gradient from a low pH (pH 5-6) mobile phase A to a high pH (pH 10-11) mobile phase B;
(viii) the pharmaceutical composition has a CEX-HPLC peak distribution with a retention time of at least 10 minutes, at least 15 minutes, at least 20 minutes, at least 25 minutes, at least 30 minutes, at least 35 minutes, at least 40 minutes, at least 45 minutes, at least 50 minutes, or at least 55 minutes; or
(ix) the pharmaceutical composition has a CEX-HPLC peak distribution with a retention time ranging from 10-55 minutes, from 20-30 minutes or from 22 to 28 minutes.

46-57. (canceled)

58. A method of making antigen-binding proteins (ABPs) for treatment of a patient exposed to HBV, comprising:
a. isolating single cells from a blood sample from a donor previously vaccinated with a Hepatitis B virus (HBV) vaccine;
b. amplifying polynucleotides from the isolated single cells, wherein each polynucleotide encodes a cognate pair of heavy chain and light chain variable regions from a single cell by overlap extension reverse transcriptase polymerase chain reaction (OE-RT-PCR);
c. cloning the polynucleotides obtained from the amplification into an expression vector, thereby obtaining polynucleotide constructs encoding recombinant antibodies;
d. introducing the antibody expression constructs into a cell line;
e. expressing antibodies from the antibody expression constructs in the cell line; and
f. obtaining the ABPs.

59-77. (canceled)

78. A plurality of ABPs generated by the method of claim 58.

79. A plurality of isolated polynucleotides, wherein each of the isolated polynucleotides encodes one of the plurality of ABPs of claim 78.

80. A plurality of polynucleotide constructs, comprising the isolated polynucleotides of claim 79 cloned into an expression vector.

81. A plurality of host cells comprising the plurality of polynucleotide constructs of claim 80.

82-83. (canceled)

84. A method of treating or preventing viral infection in a patient, comprising administering an effective amount of the pharmaceutical composition of claim 1.

85. The method of claim 84, wherein the patient:
(i) had or has a risk of exposure to Hepatitis B virus (HBV) by parenteral exposure, direct mucous membrane contact, or oral ingestion involving HBV surface antigen-positive materials such as blood, plasma, or serum;
(ii) is an infant born or will be born to an Hepatitis B virus (HBV) surface antigen-positive mother;
(iii) has Hepatitis B virus (HBV) exposure by sexual exposure to an HBV surface antigen-positive person, has household exposure to a person with acute HBV infection, chronic HBV infection, or chronic hepatitis B infection;
(iv) has HBV exposure or has a risk of HBV exposure to at least one HBV mutant by sexual exposure to a HBV mutant surface antigen-positive person, household exposure to a person with acute HBV infection; or is an infant born to an HBV mutant surface antigen-positive mother;
(v) has HBV exposure or has a risk of HBV exposure to at least one HBV mutant by sexual exposure to a HBV mutant surface antigen-positive person, household exposure to a person with acute HBV infection; or is an infant born to an HBV mutant surface antigen-positive mother: wherein the at least one HBV mutant comprises an HBV surface antigen with a mutation selected from: P120Q, T126S, Q129H, M133D, M133T, F134S, K141E, P142L, P142S, D144A, D144N, and G145R;
(vi) has HBV exposure or has a risk of HBV exposure to at least one HBV mutant by sexual exposure to a HBV mutant surface antigen-positive person, household exposure to a person with acute HBV infection; or is an infant born to an HBV mutant surface antigen-positive mother, wherein the at least one HBV mutant comprises at least two HBV mutants, wherein the at least two mutants comprise an HBV surface antigen with mutations G145R and P142L;
(vii) the patient has been exposed to or has a risk of exposure to at least one HBV genotype selected from: HBV genotype A, HBV genotype B, HBV genotype C, HBV genotype D, HBV genotype E, HBV genotype F, HBV genotype G, HBV genotype H, and HBV genotype I;
(viii) the patient has been exposed to or has a risk of exposure to HBV genotype A, B, C, or D; or
(ix) wherein the patient has chronic HBV infection, optionally wherein the patient has:
HBeAg negative chronic HBV infection for more than 6 months,
HBsAg in serum for more than 6 months,
HBsAg concentration in serum between ≥100 IU/mL and ≤2000 IU/mL at screening,
received stable dose of NAs for over 6 months, and/or
serum HBV DNA concentration not higher than 50 IU/mL at screening.

86-111. (canceled)

112. A drug product comprising the pharmaceutical composition of claim 1.

113. A unit dose of the pharmaceutical composition of claim 1.
